SEQUENCE LISTING (96 sequences)

SEQ ID NO: 1 - protein, 837 aa, Homo sapiens
MAAPGAPAEYGYIRTVLGQQILGQLDSSSLALPSEAKLKLAGSSGRGGQTVKSLRIQEQVQQTLARKGRSSVGNGNLHRTSSVPEYVYNLHLVENDFVGGRSPVPKTYDMLKAGTTATYEGRWGRGTAQYSSQKSVEERSLRHPLRRLEISPDSSPERAHYTHSDYQYSQRSQAGHTLHHQESRRAALLVPPRYARSEIVGVSRAGTTSRQRHFDTYHRQYQHGSVSDTVFDSIPANPALLTYPRPGTSRSMGNLLEKENYLTAGLTVGQVRPLVPLQPVTQNRASRSSWHQSSFHSTRTLREAGPSVAVDSSGRRAHLTVGQAAAGGSGNLLTERSTFTDSQLGNADMEMTLERAVSMLEADHMLPSRISAAATFIQHECFQKSEARKRVNQLRGILKLLQLLKVQNEDVQRAVCGALRNLVFEDNDNKLEVAELNGVPRLLQVLKQTRDLETKKQITGLLWNLSSNDKLKNLMITEALLTLTENIIIPFSGWPEGDYPKANGLLDFDIFYNVTGCLRNMSSAGADGRKAMRRCDGLIDSLVHYVRGTIADYQPDDKATENCVCILHNLSYQLEAELPEKYSQNIYIQNRNIQTDNNKSIGCFGSRSRKVKEQYQDVPMPEEKSNPKGVEWLWHSIVIRMYLSLIAKSVRNYTQEASLGALQNLTAGSGPMPTSVAQTVVQKESGLQHTRKMLHVGDPSVKKTAISLLRNLSRNLSLQNEIAKETLPDLVSIIPDTVPSTDLLIETTASACYTLNNIIQNSYQNARDLLNTGGIQKIMAISAGDAYASNKASKAASVLLYSLWAHTELHHAYKKAQFKKTDFVNSRTAKAYHSLKD

SEQ ID NO: 2 - protein, 881 aa, Homo sapiens
MAAPGAPAEYGYIRTVLGQQILGQLDSSSLALPSEAKLKLAGSSGRGGQTVKSLRIQEQVQQTLARKGRSSVGNGNLHRTSSVPEYVYNLHLVENDFVGGRSPVPKTYDMLKAGTTATYEGRWGRGTAQYSSQKSVEERSLRHPLRRLEISPDSSPERAHYTHSDYQYSQRSQAGHTLHHQESRRAALLVPPRYARSEIVGVSRAGTTSRQRHFDTYHRQYQHGSVSDTVFDSIPANPALLTYPRPGTSRSMGNLLEKENYLTAGLTVGQVRPLVPLQPVTQNRASRSSWHQSSFHSTRTLREAGPSVAVDSSGRRAHLTVGQAAAGGSGNLLTERSTFTDSQLGNADMEMTLERAVSMLEADHMLPSRISAAATFIQHECFQKSEARKRVNQLRGILKLLQLLKVQNEDVQRAVCGALRNLVFEDNDNKLEVAELNGVPRLLQVLKQTRDLETKKQITDHTVNLRSRNGWPGAVAHACNPSTLGGQGGRITRSGVRDQPDQHGLLWNLSSNDKLKNLMITEALLTLTENIIIPFSGWPEGDYPKANGLLDFDIFYNVTGCLRNMSSAGADGRKAMRRCDGLIDSLVHYVRGTIADYQPDDKATENCVCILHNLSYQLEAELPEKYSQNIYIQNRNIQTDNNKSIGCFGSRSRKVKEQYQDVPMPEEKSNPKGVEWLWHSIVIRMYLSLIAKSVRNYTQEASLGALQNLTAGSGPMPTSVAQTVVQKESGLQHTRKMLHVGDPSVKKTAISLLRNLSRNLSLQNEIAKETLPDLVSIIPDTVPSTDLLIETTASACYTLNNIIQNSYQNARDLLNTGGIQKIMAISAGDAYASNKASKAASVLLYSLWAHTELHHAYKKAQFKKTDFVNSRTAKAYHSLKD

SEQ ID NO: 3 - DNA, 2511 bp, Homo sapiens
  source 1..2511: mol_type = genomic DNA
atggcagcccccggcgccccagctgagtacggctacatccggaccgtcctgggccagcagatcctgggacaactggacagctccagcctggcgctgccctccgaggccaagctgaagctggcggggagcagcggccgcggcggccagacagtcaagagcctgcggatccaggagcaggtgcagcagaccctcgcccggaagggccgcagctccgtgggcaacggaaatcttcaccgaaccagcagtgttcctgagtatgtctacaacctacacttggttgaaaatgattttgttggaggccgttcccctgttcctaaaacctatgacatgctaaaggctggcacaactgccacttatgaaggtcgctggggaagaggaacagcacagtacagctcccagaagtccgtggaagaaaggtccttgaggcatcctctgaggagactggagatttctcctgacagcagcccggagagggctcactacacgcacagcgattaccagtacagccagagaagccaggctgggcacaccctgcaccaccaagaaagcaggcgggccgccctcctagtgccaccgagatatgctcgttccgagatcgtgggggtcagccgtgctggcaccacaagcaggcagcgccactttgacacataccacagacagtaccagcatggctctgttagcgacaccgtttttgacagcatccctgccaacccggccctgctcacgtaccccaggccagggaccagccgcagcatgggcaacctcttggagaaggagaactacctgacggcagggctcactgtcgggcaggtcaggccgctggtgcccctgcagcccgtcactcagaacagggcttccaggtcctcctggcatcagagctccttccacagcacccgcacgctgagggaagctgggcccagtgtcgccgtggattccagcgggaggagagcgcacttgactgtcggccaggcggccgcagggggaagtgggaatctgctcactgagagaagcactttcactgactcccagctggggaatgcagacatggagatgactctggagcgagcagtgagtatgctcgaggcagaccacatgctgccatccaggatttctgctgcagctactttcatacagcacgagtgcttccagaaatctgaagctcggaagagggttaaccagcttcgtggcatcctcaagcttctgcagctcctaaaagttcagaatgaagacgttcagcgagctgtgtgtggggccttgagaaacttagtatttgaagacaatgacaacaaattggaggtggctgaactaaatggggtacctcggctgctccaggtgctgaagcaaaccagagacttggagactaaaaaacaaataacaggtttgctgtggaatttgtcatctaatgacaaactcaagaatctcatgataacagaagcattgcttacgctgacggagaatatcatcatccccttttctgggtggcctgaaggagactacccaaaagcaaatggtttgctcgattttgacatattctacaacgtcactggatgcctaagaaacatgagttctgctggcgctgatgggagaaaagcgatgagaagatgtgacggactcattgactcactggtccattatgtcagaggaaccattgcagattaccagccagatgacaaggccacggagaattgtgtgtgcattcttcataacctctcctaccagctggaggcagagctcccagagaaatattcccagaatatctatattcaaaaccggaatatccagactgacaacaacaaaagtattggatgttttggcagtcgaagcaggaaagtaaaagagcaataccaggacgtgccgatgccggaggaaaagagcaaccccaagggcgtggagtggctgtggcattccattgttataaggatgtatctgtccttgatcgccaaaagtgtccgcaactacacacaagaagcatccttaggagctctgcagaacctcacggccggaagtggaccaatgccgacatcagtggctcagacagttgtccagaaggaaagtggcctgcagcacacccgaaagatgctgcatgttggtgacccaagtgtgaaaaagacagccatctcgctgctgaggaatctgtcccggaatctttctctgcagaatgaaattgccaaagaaactctccctgatttggtttccatcattcctgacacagtcccgagtactgaccttctcattgaaactacagcctctgcctgttacacattgaacaacataatccaaaacagttaccagaatgcacgcgaccttctaaacaccgggggcatccagaaaattatggccattagtgcaggcgatgcctatgcctccaacaaagcaagtaaagctgcttccgtccttctgtattctctgtgggcacacacggaactgcatcatgcctacaagaaggctcagtttaagaagacagattttgtcaacagccggactgccaaagcctaccactcccttaaagac

SEQ ID NO: 4 - DNA, 2643 bp, Homo sapiens
  source 1..2643: mol_type = genomic DNA
atggcagcccccggcgccccagctgagtacggctacatccggaccgtcctgggccagcagatcctgggacaactggacagctccagcctggcgctgccctccgaggccaagctgaagctggcggggagcagcggccgcggcggccagacagtcaagagcctgcggatccaggagcaggtgcagcagaccctcgcccggaagggccgcagctccgtgggcaacggaaatcttcaccgaaccagcagtgttcctgagtatgtctacaacctacacttggttgaaaatgattttgttggaggccgttcccctgttcctaaaacctatgacatgctaaaggctggcacaactgccacttatgaaggtcgctggggaagaggaacagcacagtacagctcccagaagtccgtggaagaaaggtccttgaggcatcctctgaggagactggagatttctcctgacagcagcccggagagggctcactacacgcacagcgattaccagtacagccagagaagccaggctgggcacaccctgcaccaccaagaaagcaggcgggccgccctcctagtgccaccgagatatgctcgttccgagatcgtgggggtcagccgtgctggcaccacaagcaggcagcgccactttgacacataccacagacagtaccagcatggctctgttagcgacaccgtttttgacagcatccctgccaacccggccctgctcacgtaccccaggccagggaccagccgcagcatgggcaacctcttggagaaggagaactacctgacggcagggctcactgtcgggcaggtcaggccgctggtgcccctgcagcccgtcactcagaacagggcttccaggtcctcctggcatcagagctccttccacagcacccgcacgctgagggaagctgggcccagtgtcgccgtggattccagcgggaggagagcgcacttgactgtcggccaggcggccgcagggggaagtgggaatctgctcactgagagaagcactttcactgactcccagctggggaatgcagacatggagatgactctggagcgagcagtgagtatgctcgaggcagaccacatgctgccatccaggatttctgctgcagctactttcatacagcacgagtgcttccagaaatctgaagctcggaagagggttaaccagcttcgtggcatcctcaagcttctgcagctcctaaaagttcagaatgaagacgttcagcgagctgtgtgtggggccttgagaaacttagtatttgaagacaatgacaacaaattggaggtggctgaactaaatggggtacctcggctgctccaggtgctgaagcaaaccagagacttggagactaaaaaacaaataacagaccatacagtcaatttaagaagtaggaatggctggccgggcgcggtggctcacgcctgtaatcccagcactttgggaggccaaggcgggcggatcacgaggtcaggagttcgagaccagcctgaccaacatggtttgctgtggaatttgtcatctaatgacaaactcaagaatctcatgataacagaagcattgcttacgctgacggagaatatcatcatccccttttctgggtggcctgaaggagactacccaaaagcaaatggtttgctcgattttgacatattctacaacgtcactggatgcctaagaaacatgagttctgctggcgctgatgggagaaaagcgatgagaagatgtgacggactcattgactcactggtccattatgtcagaggaaccattgcagattaccagccagatgacaaggccacggagaattgtgtgtgcattcttcataacctctcctaccagctggaggcagagctcccagagaaatattcccagaatatctatattcaaaaccggaatatccagactgacaacaacaaaagtattggatgttttggcagtcgaagcaggaaagtaaaagagcaataccaggacgtgccgatgccggaggaaaagagcaaccccaagggcgtggagtggctgtggcattccattgttataaggatgtatctgtccttgatcgccaaaagtgtccgcaactacacacaagaagcatccttaggagctctgcagaacctcacggccggaagtggaccaatgccgacatcagtggctcagacagttgtccagaaggaaagtggcctgcagcacacccgaaagatgctgcatgttggtgacccaagtgtgaaaaagacagccatctcgctgctgaggaatctgtcccggaatctttctctgcagaatgaaattgccaaagaaactctccctgatttggtttccatcattcctgacacagtcccgagtactgaccttctcattgaaactacagcctctgcctgttacacattgaacaacataatccaaaacagttaccagaatgcacgcgaccttctaaacaccgggggcatccagaaaattatggccattagtgcaggcgatgcctatgcctccaacaaagcaagtaaagctgcttccgtccttctgtattctctgtgggcacacacggaactgcatcatgcctacaagaaggctcagtttaagaagacagattttgtcaacagccggactgccaaagcctaccactcccttaaagac

SEQ ID NO: 5 - DNA, 10 bp, synthetic construct
  misc_feature 1..10: note = Kozak sequence motif
  source 1..10: mol_type = other DNA
gccaccatgg

SEQ ID NO: 6 - DNA, 2517 bp, synthetic construct
  misc_feature 1..2517: note = Polynucleotide encoding an PKP2a with Kozak sequence
  source 1..2517: mol_type = other DNA
gccaccatggcagcccccggcgccccagctgagtacggctacatccggaccgtcctgggccagcagatcctgggacaactggacagctccagcctggcgctgccctccgaggccaagctgaagctggcggggagcagcggccgcggcggccagacagtcaagagcctgcggatccaggagcaggtgcagcagaccctcgcccggaagggccgcagctccgtgggcaacggaaatcttcaccgaaccagcagtgttcctgagtatgtctacaacctacacttggttgaaaatgattttgttggaggccgttcccctgttcctaaaacctatgacatgctaaaggctggcacaactgccacttatgaaggtcgctggggaagaggaacagcacagtacagctcccagaagtccgtggaagaaaggtccttgaggcatcctctgaggagactggagatttctcctgacagcagcccggagagggctcactacacgcacagcgattaccagtacagccagagaagccaggctgggcacaccctgcaccaccaagaaagcaggcgggccgccctcctagtgccaccgagatatgctcgttccgagatcgtgggggtcagccgtgctggcaccacaagcaggcagcgccactttgacacataccacagacagtaccagcatggctctgttagcgacaccgtttttgacagcatccctgccaacccggccctgctcacgtaccccaggccagggaccagccgcagcatgggcaacctcttggagaaggagaactacctgacggcagggctcactgtcgggcaggtcaggccgctggtgcccctgcagcccgtcactcagaacagggcttccaggtcctcctggcatcagagctccttccacagcacccgcacgctgagggaagctgggcccagtgtcgccgtggattccagcgggaggagagcgcacttgactgtcggccaggcggccgcagggggaagtgggaatctgctcactgagagaagcactttcactgactcccagctggggaatgcagacatggagatgactctggagcgagcagtgagtatgctcgaggcagaccacatgctgccatccaggatttctgctgcagctactttcatacagcacgagtgcttccagaaatctgaagctcggaagagggttaaccagcttcgtggcatcctcaagcttctgcagctcctaaaagttcagaatgaagacgttcagcgagctgtgtgtggggccttgagaaacttagtatttgaagacaatgacaacaaattggaggtggctgaactaaatggggtacctcggctgctccaggtgctgaagcaaaccagagacttggagactaaaaaacaaataacaggtttgctgtggaatttgtcatctaatgacaaactcaagaatctcatgataacagaagcattgcttacgctgacggagaatatcatcatccccttttctgggtggcctgaaggagactacccaaaagcaaatggtttgctcgattttgacatattctacaacgtcactggatgcctaagaaacatgagttctgctggcgctgatgggagaaaagcgatgagaagatgtgacggactcattgactcactggtccattatgtcagaggaaccattgcagattaccagccagatgacaaggccacggagaattgtgtgtgcattcttcataacctctcctaccagctggaggcagagctcccagagaaatattcccagaatatctatattcaaaaccggaatatccagactgacaacaacaaaagtattggatgttttggcagtcgaagcaggaaagtaaaagagcaataccaggacgtgccgatgccggaggaaaagagcaaccccaagggcgtggagtggctgtggcattccattgttataaggatgtatctgtccttgatcgccaaaagtgtccgcaactacacacaagaagcatccttaggagctctgcagaacctcacggccggaagtggaccaatgccgacatcagtggctcagacagttgtccagaaggaaagtggcctgcagcacacccgaaagatgctgcatgttggtgacccaagtgtgaaaaagacagccatctcgctgctgaggaatctgtcccggaatctttctctgcagaatgaaattgccaaagaaactctccctgatttggtttccatcattcctgacacagtcccgagtactgaccttctcattgaaactacagcctctgcctgttacacattgaacaacataatccaaaacagttaccagaatgcacgcgaccttctaaacaccgggggcatccagaaaattatggccattagtgcaggcgatgcctatgcctccaacaaagcaagtaaagctgcttccgtccttctgtattctctgtgggcacacacggaactgcatcatgcctacaagaaggctcagtttaagaagacagattttgtcaacagccggactgccaaagcctaccactcccttaaagac

SEQ ID NO: 7 - DNA, 2649 bp, synthetic construct
  misc_feature 1..2649: note = Polynucleotide encoding an PKP2b with Kozak sequence
  source 1..2649: mol_type = other DNA
gccaccatggcagcccccggcgccccagctgagtacggctacatccggaccgtcctgggccagcagatcctgggacaactggacagctccagcctggcgctgccctccgaggccaagctgaagctggcggggagcagcggccgcggcggccagacagtcaagagcctgcggatccaggagcaggtgcagcagaccctcgcccggaagggccgcagctccgtgggcaacggaaatcttcaccgaaccagcagtgttcctgagtatgtctacaacctacacttggttgaaaatgattttgttggaggccgttcccctgttcctaaaacctatgacatgctaaaggctggcacaactgccacttatgaaggtcgctggggaagaggaacagcacagtacagctcccagaagtccgtggaagaaaggtccttgaggcatcctctgaggagactggagatttctcctgacagcagcccggagagggctcactacacgcacagcgattaccagtacagccagagaagccaggctgggcacaccctgcaccaccaagaaagcaggcgggccgccctcctagtgccaccgagatatgctcgttccgagatcgtgggggtcagccgtgctggcaccacaagcaggcagcgccactttgacacataccacagacagtaccagcatggctctgttagcgacaccgtttttgacagcatccctgccaacccggccctgctcacgtaccccaggccagggaccagccgcagcatgggcaacctcttggagaaggagaactacctgacggcagggctcactgtcgggcaggtcaggccgctggtgcccctgcagcccgtcactcagaacagggcttccaggtcctcctggcatcagagctccttccacagcacccgcacgctgagggaagctgggcccagtgtcgccgtggattccagcgggaggagagcgcacttgactgtcggccaggcggccgcagggggaagtgggaatctgctcactgagagaagcactttcactgactcccagctggggaatgcagacatggagatgactctggagcgagcagtgagtatgctcgaggcagaccacatgctgccatccaggatttctgctgcagctactttcatacagcacgagtgcttccagaaatctgaagctcggaagagggttaaccagcttcgtggcatcctcaagcttctgcagctcctaaaagttcagaatgaagacgttcagcgagctgtgtgtggggccttgagaaacttagtatttgaagacaatgacaacaaattggaggtggctgaactaaatggggtacctcggctgctccaggtgctgaagcaaaccagagacttggagactaaaaaacaaataacagaccatacagtcaatttaagaagtaggaatggctggccgggcgcggtggctcacgcctgtaatcccagcactttgggaggccaaggcgggcggatcacgaggtcaggagttcgagaccagcctgaccaacatggtttgctgtggaatttgtcatctaatgacaaactcaagaatctcatgataacagaagcattgcttacgctgacggagaatatcatcatccccttttctgggtggcctgaaggagactacccaaaagcaaatggtttgctcgattttgacatattctacaacgtcactggatgcctaagaaacatgagttctgctggcgctgatgggagaaaagcgatgagaagatgtgacggactcattgactcactggtccattatgtcagaggaaccattgcagattaccagccagatgacaaggccacggagaattgtgtgtgcattcttcataacctctcctaccagctggaggcagagctcccagagaaatattcccagaatatctatattcaaaaccggaatatccagactgacaacaacaaaagtattggatgttttggcagtcgaagcaggaaagtaaaagagcaataccaggacgtgccgatgccggaggaaaagagcaaccccaagggcgtggagtggctgtggcattccattgttataaggatgtatctgtccttgatcgccaaaagtgtccgcaactacacacaagaagcatccttaggagctctgcagaacctcacggccggaagtggaccaatgccgacatcagtggctcagacagttgtccagaaggaaagtggcctgcagcacacccgaaagatgctgcatgttggtgacccaagtgtgaaaaagacagccatctcgctgctgaggaatctgtcccggaatctttctctgcagaatgaaattgccaaagaaactctccctgatttggtttccatcattcctgacacagtcccgagtactgaccttctcattgaaactacagcctctgcctgttacacattgaacaacataatccaaaacagttaccagaatgcacgcgaccttctaaacaccgggggcatccagaaaattatggccattagtgcaggcgatgcctatgcctccaacaaagcaagtaaagctgcttccgtccttctgtattctctgtgggcacacacggaactgcatcatgcctacaagaaggctcagtttaagaagacagattttgtcaacagccggactgccaaagcctaccactcccttaaagac

SEQ ID NO: 8 - DNA, 4081 bp, synthetic construct
  misc_feature 1..4081: note = Made in Lab - MHCK7-PKP2a expression cassette
  source 1..4081: mol_type = other DNA
acccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccaggccaccatggcagcccccggcgccccagctgagtacggctacatccggaccgtcctgggccagcagatcctgggacaactggacagctccagcctggcgctgccctccgaggccaagctgaagctggcggggagcagcggccgcggcggccagacagtcaagagcctgcggatccaggagcaggtgcagcagaccctcgcccggaagggccgcagctccgtgggcaacggaaatcttcaccgaaccagcagtgttcctgagtatgtctacaacctacacttggttgaaaatgattttgttggaggccgttcccctgttcctaaaacctatgacatgctaaaggctggcacaactgccacttatgaaggtcgctggggaagaggaacagcacagtacagctcccagaagtccgtggaagaaaggtccttgaggcatcctctgaggagactggagatttctcctgacagcagcccggagagggctcactacacgcacagcgattaccagtacagccagagaagccaggctgggcacaccctgcaccaccaagaaagcaggcgggccgccctcctagtgccaccgagatatgctcgttccgagatcgtgggggtcagccgtgctggcaccacaagcaggcagcgccactttgacacataccacagacagtaccagcatggctctgttagcgacaccgtttttgacagcatccctgccaacccggccctgctcacgtaccccaggccagggaccagccgcagcatgggcaacctcttggagaaggagaactacctgacggcagggctcactgtcgggcaggtcaggccgctggtgcccctgcagcccgtcactcagaacagggcttccaggtcctcctggcatcagagctccttccacagcacccgcacgctgagggaagctgggcccagtgtcgccgtggattccagcgggaggagagcgcacttgactgtcggccaggcggccgcagggggaagtgggaatctgctcactgagagaagcactttcactgactcccagctggggaatgcagacatggagatgactctggagcgagcagtgagtatgctcgaggcagaccacatgctgccatccaggatttctgctgcagctactttcatacagcacgagtgcttccagaaatctgaagctcggaagagggttaaccagcttcgtggcatcctcaagcttctgcagctcctaaaagttcagaatgaagacgttcagcgagctgtgtgtggggccttgagaaacttagtatttgaagacaatgacaacaaattggaggtggctgaactaaatggggtacctcggctgctccaggtgctgaagcaaaccagagacttggagactaaaaaacaaataacaggtttgctgtggaatttgtcatctaatgacaaactcaagaatctcatgataacagaagcattgcttacgctgacggagaatatcatcatccccttttctgggtggcctgaaggagactacccaaaagcaaatggtttgctcgattttgacatattctacaacgtcactggatgcctaagaaacatgagttctgctggcgctgatgggagaaaagcgatgagaagatgtgacggactcattgactcactggtccattatgtcagaggaaccattgcagattaccagccagatgacaaggccacggagaattgtgtgtgcattcttcataacctctcctaccagctggaggcagagctcccagagaaatattcccagaatatctatattcaaaaccggaatatccagactgacaacaacaaaagtattggatgttttggcagtcgaagcaggaaagtaaaagagcaataccaggacgtgccgatgccggaggaaaagagcaaccccaagggcgtggagtggctgtggcattccattgttataaggatgtatctgtccttgatcgccaaaagtgtccgcaactacacacaagaagcatccttaggagctctgcagaacctcacggccggaagtggaccaatgccgacatcagtggctcagacagttgtccagaaggaaagtggcctgcagcacacccgaaagatgctgcatgttggtgacccaagtgtgaaaaagacagccatctcgctgctgaggaatctgtcccggaatctttctctgcagaatgaaattgccaaagaaactctccctgatttggtttccatcattcctgacacagtcccgagtactgaccttctcattgaaactacagcctctgcctgttacacattgaacaacataatccaaaacagttaccagaatgcacgcgaccttctaaacaccgggggcatccagaaaattatggccattagtgcaggcgatgcctatgcctccaacaaagcaagtaaagctgcttccgtccttctgtattctctgtgggcacacacggaactgcatcatgcctacaagaaggctcagtttaagaagacagattttgtcaacagccggactgccaaagcctaccactcccttaaagactgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgc

SEQ ID NO: 9 - DNA, 3854 bp, synthetic construct
  misc_feature 1..3854: note = Made in lab - hTnT-PKP2a expression cassette
  source 1..3854: mol_type = other DNA
ctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcaggccaccatggcagcccccggcgccccagctgagtacggctacatccggaccgtcctgggccagcagatcctgggacaactggacagctccagcctggcgctgccctccgaggccaagctgaagctggcggggagcagcggccgcggcggccagacagtcaagagcctgcggatccaggagcaggtgcagcagaccctcgcccggaagggccgcagctccgtgggcaacggaaatcttcaccgaaccagcagtgttcctgagtatgtctacaacctacacttggttgaaaatgattttgttggaggccgttcccctgttcctaaaacctatgacatgctaaaggctggcacaactgccacttatgaaggtcgctggggaagaggaacagcacagtacagctcccagaagtccgtggaagaaaggtccttgaggcatcctctgaggagactggagatttctcctgacagcagcccggagagggctcactacacgcacagcgattaccagtacagccagagaagccaggctgggcacaccctgcaccaccaagaaagcaggcgggccgccctcctagtgccaccgagatatgctcgttccgagatcgtgggggtcagccgtgctggcaccacaagcaggcagcgccactttgacacataccacagacagtaccagcatggctctgttagcgacaccgtttttgacagcatccctgccaacccggccctgctcacgtaccccaggccagggaccagccgcagcatgggcaacctcttggagaaggagaactacctgacggcagggctcactgtcgggcaggtcaggccgctggtgcccctgcagcccgtcactcagaacagggcttccaggtcctcctggcatcagagctccttccacagcacccgcacgctgagggaagctgggcccagtgtcgccgtggattccagcgggaggagagcgcacttgactgtcggccaggcggccgcagggggaagtgggaatctgctcactgagagaagcactttcactgactcccagctggggaatgcagacatggagatgactctggagcgagcagtgagtatgctcgaggcagaccacatgctgccatccaggatttctgctgcagctactttcatacagcacgagtgcttccagaaatctgaagctcggaagagggttaaccagcttcgtggcatcctcaagcttctgcagctcctaaaagttcagaatgaagacgttcagcgagctgtgtgtggggccttgagaaacttagtatttgaagacaatgacaacaaattggaggtggctgaactaaatggggtacctcggctgctccaggtgctgaagcaaaccagagacttggagactaaaaaacaaataacaggtttgctgtggaatttgtcatctaatgacaaactcaagaatctcatgataacagaagcattgcttacgctgacggagaatatcatcatccccttttctgggtggcctgaaggagactacccaaaagcaaatggtttgctcgattttgacatattctacaacgtcactggatgcctaagaaacatgagttctgctggcgctgatgggagaaaagcgatgagaagatgtgacggactcattgactcactggtccattatgtcagaggaaccattgcagattaccagccagatgacaaggccacggagaattgtgtgtgcattcttcataacctctcctaccagctggaggcagagctcccagagaaatattcccagaatatctatattcaaaaccggaatatccagactgacaacaacaaaagtattggatgttttggcagtcgaagcaggaaagtaaaagagcaataccaggacgtgccgatgccggaggaaaagagcaaccccaagggcgtggagtggctgtggcattccattgttataaggatgtatctgtccttgatcgccaaaagtgtccgcaactacacacaagaagcatccttaggagctctgcagaacctcacggccggaagtggaccaatgccgacatcagtggctcagacagttgtccagaaggaaagtggcctgcagcacacccgaaagatgctgcatgttggtgacccaagtgtgaaaaagacagccatctcgctgctgaggaatctgtcccggaatctttctctgcagaatgaaattgccaaagaaactctccctgatttggtttccatcattcctgacacagtcccgagtactgaccttctcattgaaactacagcctctgcctgttacacattgaacaacataatccaaaacagttaccagaatgcacgcgaccttctaaacaccgggggcatccagaaaattatggccattagtgcaggcgatgcctatgcctccaacaaagcaagtaaagctgcttccgtccttctgtattctctgtgggcacacacggaactgcatcatgcctacaagaaggctcagtttaagaagacagattttgtcaacagccggactgccaaagcctaccactcccttaaagactgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgc

SEQ ID NO: 10 - DNA, 4213 bp, synthetic construct
  misc_feature 1..4213: note = Made in Lab - MHCK7-PKP2b expression cassette
  source 1..4213: mol_type = other DNA
acccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccaggccaccatggcagcccccggcgccccagctgagtacggctacatccggaccgtcctgggccagcagatcctgggacaactggacagctccagcctggcgctgccctccgaggccaagctgaagctggcggggagcagcggccgcggcggccagacagtcaagagcctgcggatccaggagcaggtgcagcagaccctcgcccggaagggccgcagctccgtgggcaacggaaatcttcaccgaaccagcagtgttcctgagtatgtctacaacctacacttggttgaaaatgattttgttggaggccgttcccctgttcctaaaacctatgacatgctaaaggctggcacaactgccacttatgaaggtcgctggggaagaggaacagcacagtacagctcccagaagtccgtggaagaaaggtccttgaggcatcctctgaggagactggagatttctcctgacagcagcccggagagggctcactacacgcacagcgattaccagtacagccagagaagccaggctgggcacaccctgcaccaccaagaaagcaggcgggccgccctcctagtgccaccgagatatgctcgttccgagatcgtgggggtcagccgtgctggcaccacaagcaggcagcgccactttgacacataccacagacagtaccagcatggctctgttagcgacaccgtttttgacagcatccctgccaacccggccctgctcacgtaccccaggccagggaccagccgcagcatgggcaacctcttggagaaggagaactacctgacggcagggctcactgtcgggcaggtcaggccgctggtgcccctgcagcccgtcactcagaacagggcttccaggtcctcctggcatcagagctccttccacagcacccgcacgctgagggaagctgggcccagtgtcgccgtggattccagcgggaggagagcgcacttgactgtcggccaggcggccgcagggggaagtgggaatctgctcactgagagaagcactttcactgactcccagctggggaatgcagacatggagatgactctggagcgagcagtgagtatgctcgaggcagaccacatgctgccatccaggatttctgctgcagctactttcatacagcacgagtgcttccagaaatctgaagctcggaagagggttaaccagcttcgtggcatcctcaagcttctgcagctcctaaaagttcagaatgaagacgttcagcgagctgtgtgtggggccttgagaaacttagtatttgaagacaatgacaacaaattggaggtggctgaactaaatggggtacctcggctgctccaggtgctgaagcaaaccagagacttggagactaaaaaacaaataacagaccatacagtcaatttaagaagtaggaatggctggccgggcgcggtggctcacgcctgtaatcccagcactttgggaggccaaggcgggcggatcacgaggtcaggagttcgagaccagcctgaccaacatggtttgctgtggaatttgtcatctaatgacaaactcaagaatctcatgataacagaagcattgcttacgctgacggagaatatcatcatccccttttctgggtggcctgaaggagactacccaaaagcaaatggtttgctcgattttgacatattctacaacgtcactggatgcctaagaaacatgagttctgctggcgctgatgggagaaaagcgatgagaagatgtgacggactcattgactcactggtccattatgtcagaggaaccattgcagattaccagccagatgacaaggccacggagaattgtgtgtgcattcttcataacctctcctaccagctggaggcagagctcccagagaaatattcccagaatatctatattcaaaaccggaatatccagactgacaacaacaaaagtattggatgttttggcagtcgaagcaggaaagtaaaagagcaataccaggacgtgccgatgccggaggaaaagagcaaccccaagggcgtggagtggctgtggcattccattgttataaggatgtatctgtccttgatcgccaaaagtgtccgcaactacacacaagaagcatccttaggagctctgcagaacctcacggccggaagtggaccaatgccgacatcagtggctcagacagttgtccagaaggaaagtggcctgcagcacacccgaaagatgctgcatgttggtgacccaagtgtgaaaaagacagccatctcgctgctgaggaatctgtcccggaatctttctctgcagaatgaaattgccaaagaaactctccctgatttggtttccatcattcctgacacagtcccgagtactgaccttctcattgaaactacagcctctgcctgttacacattgaacaacataatccaaaacagttaccagaatgcacgcgaccttctaaacaccgggggcatccagaaaattatggccattagtgcaggcgatgcctatgcctccaacaaagcaagtaaagctgcttccgtccttctgtattctctgtgggcacacacggaactgcatcatgcctacaagaaggctcagtttaagaagacagattttgtcaacagccggactgccaaagcctaccactcccttaaagactgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgc

SEQ ID NO: 11 - DNA, 3986 bp, synthetic construct
  misc_feature 1..3986: note = Made in Lab - hTnT-PKP2b express cassette
  source 1..3986: mol_type = other DNA
ctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcaggccaccatggcagcccccggcgccccagctgagtacggctacatccggaccgtcctgggccagcagatcctgggacaactggacagctccagcctggcgctgccctccgaggccaagctgaagctggcggggagcagcggccgcggcggccagacagtcaagagcctgcggatccaggagcaggtgcagcagaccctcgcccggaagggccgcagctccgtgggcaacggaaatcttcaccgaaccagcagtgttcctgagtatgtctacaacctacacttggttgaaaatgattttgttggaggccgttcccctgttcctaaaacctatgacatgctaaaggctggcacaactgccacttatgaaggtcgctggggaagaggaacagcacagtacagctcccagaagtccgtggaagaaaggtccttgaggcatcctctgaggagactggagatttctcctgacagcagcccggagagggctcactacacgcacagcgattaccagtacagccagagaagccaggctgggcacaccctgcaccaccaagaaagcaggcgggccgccctcctagtgccaccgagatatgctcgttccgagatcgtgggggtcagccgtgctggcaccacaagcaggcagcgccactttgacacataccacagacagtaccagcatggctctgttagcgacaccgtttttgacagcatccctgccaacccggccctgctcacgtaccccaggccagggaccagccgcagcatgggcaacctcttggagaaggagaactacctgacggcagggctcactgtcgggcaggtcaggccgctggtgcccctgcagcccgtcactcagaacagggcttccaggtcctcctggcatcagagctccttccacagcacccgcacgctgagggaagctgggcccagtgtcgccgtggattccagcgggaggagagcgcacttgactgtcggccaggcggccgcagggggaagtgggaatctgctcactgagagaagcactttcactgactcccagctggggaatgcagacatggagatgactctggagcgagcagtgagtatgctcgaggcagaccacatgctgccatccaggatttctgctgcagctactttcatacagcacgagtgcttccagaaatctgaagctcggaagagggttaaccagcttcgtggcatcctcaagcttctgcagctcctaaaagttcagaatgaagacgttcagcgagctgtgtgtggggccttgagaaacttagtatttgaagacaatgacaacaaattggaggtggctgaactaaatggggtacctcggctgctccaggtgctgaagcaaaccagagacttggagactaaaaaacaaataacagaccatacagtcaatttaagaagtaggaatggctggccgggcgcggtggctcacgcctgtaatcccagcactttgggaggccaaggcgggcggatcacgaggtcaggagttcgagaccagcctgaccaacatggtttgctgtggaatttgtcatctaatgacaaactcaagaatctcatgataacagaagcattgcttacgctgacggagaatatcatcatccccttttctgggtggcctgaaggagactacccaaaagcaaatggtttgctcgattttgacatattctacaacgtcactggatgcctaagaaacatgagttctgctggcgctgatgggagaaaagcgatgagaagatgtgacggactcattgactcactggtccattatgtcagaggaaccattgcagattaccagccagatgacaaggccacggagaattgtgtgtgcattcttcataacctctcctaccagctggaggcagagctcccagagaaatattcccagaatatctatattcaaaaccggaatatccagactgacaacaacaaaagtattggatgttttggcagtcgaagcaggaaagtaaaagagcaataccaggacgtgccgatgccggaggaaaagagcaaccccaagggcgtggagtggctgtggcattccattgttataaggatgtatctgtccttgatcgccaaaagtgtccgcaactacacacaagaagcatccttaggagctctgcagaacctcacggccggaagtggaccaatgccgacatcagtggctcagacagttgtccagaaggaaagtggcctgcagcacacccgaaagatgctgcatgttggtgacccaagtgtgaaaaagacagccatctcgctgctgaggaatctgtcccggaatctttctctgcagaatgaaattgccaaagaaactctccctgatttggtttccatcattcctgacacagtcccgagtactgaccttctcattgaaactacagcctctgcctgttacacattgaacaacataatccaaaacagttaccagaatgcacgcgaccttctaaacaccgggggcatccagaaaattatggccattagtgcaggcgatgcctatgcctccaacaaagcaagtaaagctgcttccgtccttctgtattctctgtgggcacacacggaactgcatcatgcctacaagaaggctcagtttaagaagacagattttgtcaacagccggactgccaaagcctaccactcccttaaagactgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgc

SEQ ID NO: 12 - DNA, 4417 bp, synthetic construct
  misc_feature 1..4417: note = Made in Labe - full polynucleotide sequence of vector genome
  source 1..4417: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtaacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccaggccaccatggcagcccccggcgccccagctgagtacggctacatccggaccgtcctgggccagcagatcctgggacaactggacagctccagcctggcgctgccctccgaggccaagctgaagctggcggggagcagcggccgcggcggccagacagtcaagagcctgcggatccaggagcaggtgcagcagaccctcgcccggaagggccgcagctccgtgggcaacggaaatcttcaccgaaccagcagtgttcctgagtatgtctacaacctacacttggttgaaaatgattttgttggaggccgttcccctgttcctaaaacctatgacatgctaaaggctggcacaactgccacttatgaaggtcgctggggaagaggaacagcacagtacagctcccagaagtccgtggaagaaaggtccttgaggcatcctctgaggagactggagatttctcctgacagcagcccggagagggctcactacacgcacagcgattaccagtacagccagagaagccaggctgggcacaccctgcaccaccaagaaagcaggcgggccgccctcctagtgccaccgagatatgctcgttccgagatcgtgggggtcagccgtgctggcaccacaagcaggcagcgccactttgacacataccacagacagtaccagcatggctctgttagcgacaccgtttttgacagcatccctgccaacccggccctgctcacgtaccccaggccagggaccagccgcagcatgggcaacctcttggagaaggagaactacctgacggcagggctcactgtcgggcaggtcaggccgctggtgcccctgcagcccgtcactcagaacagggcttccaggtcctcctggcatcagagctccttccacagcacccgcacgctgagggaagctgggcccagtgtcgccgtggattccagcgggaggagagcgcacttgactgtcggccaggcggccgcagggggaagtgggaatctgctcactgagagaagcactttcactgactcccagctggggaatgcagacatggagatgactctggagcgagcagtgagtatgctcgaggcagaccacatgctgccatccaggatttctgctgcagctactttcatacagcacgagtgcttccagaaatctgaagctcggaagagggttaaccagcttcgtggcatcctcaagcttctgcagctcctaaaagttcagaatgaagacgttcagcgagctgtgtgtggggccttgagaaacttagtatttgaagacaatgacaacaaattggaggtggctgaactaaatggggtacctcggctgctccaggtgctgaagcaaaccagagacttggagactaaaaaacaaataacaggtttgctgtggaatttgtcatctaatgacaaactcaagaatctcatgataacagaagcattgcttacgctgacggagaatatcatcatccccttttctgggtggcctgaaggagactacccaaaagcaaatggtttgctcgattttgacatattctacaacgtcactggatgcctaagaaacatgagttctgctggcgctgatgggagaaaagcgatgagaagatgtgacggactcattgactcactggtccattatgtcagaggaaccattgcagattaccagccagatgacaaggccacggagaattgtgtgtgcattcttcataacctctcctaccagctggaggcagagctcccagagaaatattcccagaatatctatattcaaaaccggaatatccagactgacaacaacaaaagtattggatgttttggcagtcgaagcaggaaagtaaaagagcaataccaggacgtgccgatgccggaggaaaagagcaaccccaagggcgtggagtggctgtggcattccattgttataaggatgtatctgtccttgatcgccaaaagtgtccgcaactacacacaagaagcatccttaggagctctgcagaacctcacggccggaagtggaccaatgccgacatcagtggctcagacagttgtccagaaggaaagtggcctgcagcacacccgaaagatgctgcatgttggtgacccaagtgtgaaaaagacagccatctcgctgctgaggaatctgtcccggaatctttctctgcagaatgaaattgccaaagaaactctccctgatttggtttccatcattcctgacacagtcccgagtactgaccttctcattgaaactacagcctctgcctgttacacattgaacaacataatccaaaacagttaccagaatgcacgcgaccttctaaacaccgggggcatccagaaaattatggccattagtgcaggcgatgcctatgcctccaacaaagcaagtaaagctgcttccgtccttctgtattctctgtgggcacacacggaactgcatcatgcctacaagaaggctcagtttaagaagacagattttgtcaacagccggactgccaaagcctaccactcccttaaagactgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 13 - DNA, 4190 bp, synthetic construct
  misc_feature 1..4190: note = Made in Labe - full polynucleotide sequence of vector genome
  source 1..4190: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcaggccaccatggcagcccccggcgccccagctgagtacggctacatccggaccgtcctgggccagcagatcctgggacaactggacagctccagcctggcgctgccctccgaggccaagctgaagctggcggggagcagcggccgcggcggccagacagtcaagagcctgcggatccaggagcaggtgcagcagaccctcgcccggaagggccgcagctccgtgggcaacggaaatcttcaccgaaccagcagtgttcctgagtatgtctacaacctacacttggttgaaaatgattttgttggaggccgttcccctgttcctaaaacctatgacatgctaaaggctggcacaactgccacttatgaaggtcgctggggaagaggaacagcacagtacagctcccagaagtccgtggaagaaaggtccttgaggcatcctctgaggagactggagatttctcctgacagcagcccggagagggctcactacacgcacagcgattaccagtacagccagagaagccaggctgggcacaccctgcaccaccaagaaagcaggcgggccgccctcctagtgccaccgagatatgctcgttccgagatcgtgggggtcagccgtgctggcaccacaagcaggcagcgccactttgacacataccacagacagtaccagcatggctctgttagcgacaccgtttttgacagcatccctgccaacccggccctgctcacgtaccccaggccagggaccagccgcagcatgggcaacctcttggagaaggagaactacctgacggcagggctcactgtcgggcaggtcaggccgctggtgcccctgcagcccgtcactcagaacagggcttccaggtcctcctggcatcagagctccttccacagcacccgcacgctgagggaagctgggcccagtgtcgccgtggattccagcgggaggagagcgcacttgactgtcggccaggcggccgcagggggaagtgggaatctgctcactgagagaagcactttcactgactcccagctggggaatgcagacatggagatgactctggagcgagcagtgagtatgctcgaggcagaccacatgctgccatccaggatttctgctgcagctactttcatacagcacgagtgcttccagaaatctgaagctcggaagagggttaaccagcttcgtggcatcctcaagcttctgcagctcctaaaagttcagaatgaagacgttcagcgagctgtgtgtggggccttgagaaacttagtatttgaagacaatgacaacaaattggaggtggctgaactaaatggggtacctcggctgctccaggtgctgaagcaaaccagagacttggagactaaaaaacaaataacaggtttgctgtggaatttgtcatctaatgacaaactcaagaatctcatgataacagaagcattgcttacgctgacggagaatatcatcatccccttttctgggtggcctgaaggagactacccaaaagcaaatggtttgctcgattttgacatattctacaacgtcactggatgcctaagaaacatgagttctgctggcgctgatgggagaaaagcgatgagaagatgtgacggactcattgactcactggtccattatgtcagaggaaccattgcagattaccagccagatgacaaggccacggagaattgtgtgtgcattcttcataacctctcctaccagctggaggcagagctcccagagaaatattcccagaatatctatattcaaaaccggaatatccagactgacaacaacaaaagtattggatgttttggcagtcgaagcaggaaagtaaaagagcaataccaggacgtgccgatgccggaggaaaagagcaaccccaagggcgtggagtggctgtggcattccattgttataaggatgtatctgtccttgatcgccaaaagtgtccgcaactacacacaagaagcatccttaggagctctgcagaacctcacggccggaagtggaccaatgccgacatcagtggctcagacagttgtccagaaggaaagtggcctgcagcacacccgaaagatgctgcatgttggtgacccaagtgtgaaaaagacagccatctcgctgctgaggaatctgtcccggaatctttctctgcagaatgaaattgccaaagaaactctccctgatttggtttccatcattcctgacacagtcccgagtactgaccttctcattgaaactacagcctctgcctgttacacattgaacaacataatccaaaacagttaccagaatgcacgcgaccttctaaacaccgggggcatccagaaaattatggccattagtgcaggcgatgcctatgcctccaacaaagcaagtaaagctgcttccgtccttctgtattctctgtgggcacacacggaactgcatcatgcctacaagaaggctcagtttaagaagacagattttgtcaacagccggactgccaaagcctaccactcccttaaagactgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 14 - DNA, 4549 bp, synthetic construct
  misc_feature 1..4549: note = Made in Labe - full polynucleotide sequence of vector genome
  source 1..4549: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtaacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccaggccaccatggcagcccccggcgccccagctgagtacggctacatccggaccgtcctgggccagcagatcctgggacaactggacagctccagcctggcgctgccctccgaggccaagctgaagctggcggggagcagcggccgcggcggccagacagtcaagagcctgcggatccaggagcaggtgcagcagaccctcgcccggaagggccgcagctccgtgggcaacggaaatcttcaccgaaccagcagtgttcctgagtatgtctacaacctacacttggttgaaaatgattttgttggaggccgttcccctgttcctaaaacctatgacatgctaaaggctggcacaactgccacttatgaaggtcgctggggaagaggaacagcacagtacagctcccagaagtccgtggaagaaaggtccttgaggcatcctctgaggagactggagatttctcctgacagcagcccggagagggctcactacacgcacagcgattaccagtacagccagagaagccaggctgggcacaccctgcaccaccaagaaagcaggcgggccgccctcctagtgccaccgagatatgctcgttccgagatcgtgggggtcagccgtgctggcaccacaagcaggcagcgccactttgacacataccacagacagtaccagcatggctctgttagcgacaccgtttttgacagcatccctgccaacccggccctgctcacgtaccccaggccagggaccagccgcagcatgggcaacctcttggagaaggagaactacctgacggcagggctcactgtcgggcaggtcaggccgctggtgcccctgcagcccgtcactcagaacagggcttccaggtcctcctggcatcagagctccttccacagcacccgcacgctgagggaagctgggcccagtgtcgccgtggattccagcgggaggagagcgcacttgactgtcggccaggcggccgcagggggaagtgggaatctgctcactgagagaagcactttcactgactcccagctggggaatgcagacatggagatgactctggagcgagcagtgagtatgctcgaggcagaccacatgctgccatccaggatttctgctgcagctactttcatacagcacgagtgcttccagaaatctgaagctcggaagagggttaaccagcttcgtggcatcctcaagcttctgcagctcctaaaagttcagaatgaagacgttcagcgagctgtgtgtggggccttgagaaacttagtatttgaagacaatgacaacaaattggaggtggctgaactaaatggggtacctcggctgctccaggtgctgaagcaaaccagagacttggagactaaaaaacaaataacagaccatacagtcaatttaagaagtaggaatggctggccgggcgcggtggctcacgcctgtaatcccagcactttgggaggccaaggcgggcggatcacgaggtcaggagttcgagaccagcctgaccaacatggtttgctgtggaatttgtcatctaatgacaaactcaagaatctcatgataacagaagcattgcttacgctgacggagaatatcatcatccccttttctgggtggcctgaaggagactacccaaaagcaaatggtttgctcgattttgacatattctacaacgtcactggatgcctaagaaacatgagttctgctggcgctgatgggagaaaagcgatgagaagatgtgacggactcattgactcactggtccattatgtcagaggaaccattgcagattaccagccagatgacaaggccacggagaattgtgtgtgcattcttcataacctctcctaccagctggaggcagagctcccagagaaatattcccagaatatctatattcaaaaccggaatatccagactgacaacaacaaaagtattggatgttttggcagtcgaagcaggaaagtaaaagagcaataccaggacgtgccgatgccggaggaaaagagcaaccccaagggcgtggagtggctgtggcattccattgttataaggatgtatctgtccttgatcgccaaaagtgtccgcaactacacacaagaagcatccttaggagctctgcagaacctcacggccggaagtggaccaatgccgacatcagtggctcagacagttgtccagaaggaaagtggcctgcagcacacccgaaagatgctgcatgttggtgacccaagtgtgaaaaagacagccatctcgctgctgaggaatctgtcccggaatctttctctgcagaatgaaattgccaaagaaactctccctgatttggtttccatcattcctgacacagtcccgagtactgaccttctcattgaaactacagcctctgcctgttacacattgaacaacataatccaaaacagttaccagaatgcacgcgaccttctaaacaccgggggcatccagaaaattatggccattagtgcaggcgatgcctatgcctccaacaaagcaagtaaagctgcttccgtccttctgtattctctgtgggcacacacggaactgcatcatgcctacaagaaggctcagtttaagaagacagattttgtcaacagccggactgccaaagcctaccactcccttaaagactgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 15 - DNA, 4322 bp, synthetic construct
  misc_feature 1..4322: note = Made in Labe - full polynucleotide sequence of vector genome
  source 1..4322: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcaggccaccatggcagcccccggcgccccagctgagtacggctacatccggaccgtcctgggccagcagatcctgggacaactggacagctccagcctggcgctgccctccgaggccaagctgaagctggcggggagcagcggccgcggcggccagacagtcaagagcctgcggatccaggagcaggtgcagcagaccctcgcccggaagggccgcagctccgtgggcaacggaaatcttcaccgaaccagcagtgttcctgagtatgtctacaacctacacttggttgaaaatgattttgttggaggccgttcccctgttcctaaaacctatgacatgctaaaggctggcacaactgccacttatgaaggtcgctggggaagaggaacagcacagtacagctcccagaagtccgtggaagaaaggtccttgaggcatcctctgaggagactggagatttctcctgacagcagcccggagagggctcactacacgcacagcgattaccagtacagccagagaagccaggctgggcacaccctgcaccaccaagaaagcaggcgggccgccctcctagtgccaccgagatatgctcgttccgagatcgtgggggtcagccgtgctggcaccacaagcaggcagcgccactttgacacataccacagacagtaccagcatggctctgttagcgacaccgtttttgacagcatccctgccaacccggccctgctcacgtaccccaggccagggaccagccgcagcatgggcaacctcttggagaaggagaactacctgacggcagggctcactgtcgggcaggtcaggccgctggtgcccctgcagcccgtcactcagaacagggcttccaggtcctcctggcatcagagctccttccacagcacccgcacgctgagggaagctgggcccagtgtcgccgtggattccagcgggaggagagcgcacttgactgtcggccaggcggccgcagggggaagtgggaatctgctcactgagagaagcactttcactgactcccagctggggaatgcagacatggagatgactctggagcgagcagtgagtatgctcgaggcagaccacatgctgccatccaggatttctgctgcagctactttcatacagcacgagtgcttccagaaatctgaagctcggaagagggttaaccagcttcgtggcatcctcaagcttctgcagctcctaaaagttcagaatgaagacgttcagcgagctgtgtgtggggccttgagaaacttagtatttgaagacaatgacaacaaattggaggtggctgaactaaatggggtacctcggctgctccaggtgctgaagcaaaccagagacttggagactaaaaaacaaataacagaccatacagtcaatttaagaagtaggaatggctggccgggcgcggtggctcacgcctgtaatcccagcactttgggaggccaaggcgggcggatcacgaggtcaggagttcgagaccagcctgaccaacatggtttgctgtggaatttgtcatctaatgacaaactcaagaatctcatgataacagaagcattgcttacgctgacggagaatatcatcatccccttttctgggtggcctgaaggagactacccaaaagcaaatggtttgctcgattttgacatattctacaacgtcactggatgcctaagaaacatgagttctgctggcgctgatgggagaaaagcgatgagaagatgtgacggactcattgactcactggtccattatgtcagaggaaccattgcagattaccagccagatgacaaggccacggagaattgtgtgtgcattcttcataacctctcctaccagctggaggcagagctcccagagaaatattcccagaatatctatattcaaaaccggaatatccagactgacaacaacaaaagtattggatgttttggcagtcgaagcaggaaagtaaaagagcaataccaggacgtgccgatgccggaggaaaagagcaaccccaagggcgtggagtggctgtggcattccattgttataaggatgtatctgtccttgatcgccaaaagtgtccgcaactacacacaagaagcatccttaggagctctgcagaacctcacggccggaagtggaccaatgccgacatcagtggctcagacagttgtccagaaggaaagtggcctgcagcacacccgaaagatgctgcatgttggtgacccaagtgtgaaaaagacagccatctcgctgctgaggaatctgtcccggaatctttctctgcagaatgaaattgccaaagaaactctccctgatttggtttccatcattcctgacacagtcccgagtactgaccttctcattgaaactacagcctctgcctgttacacattgaacaacataatccaaaacagttaccagaatgcacgcgaccttctaaacaccgggggcatccagaaaattatggccattagtgcaggcgatgcctatgcctccaacaaagcaagtaaagctgcttccgtccttctgtattctctgtgggcacacacggaactgcatcatgcctacaagaaggctcagtttaagaagacagattttgtcaacagccggactgccaaagcctaccactcccttaaagactgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 16 - DNA, 13 bp, synthetic construct
  misc_feature 1..13: note = Kozak sequence motif
  source 1..13: mol_type = other DNA
gccgccrccatgg

SEQ ID NO: 17 - skipped

SEQ ID NO: 18 - DNA, 10 bp, synthetic construct
  misc_feature 1..10: note = Kozak sequence motif
  source 1..10: mol_type = other DNA
gacaccatgg

SEQ ID NO: 19 - skipped

SEQ ID NO: 20 - DNA, 141 bp, unidentified
  misc_feature 1..141: note = Adeno-associated virus
  source 1..141: mol_type = genomic DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 21 - DNA, 168 bp, unidentified
  misc_feature 1..168: note = Adeno-associated virus 2
  source 1..168: mol_type = genomic DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgta

SEQ ID NO: 22 - DNA, 170 bp, unidentified
  misc_feature 1..170: note = Adeno-associated virus
  source 1..170: mol_type = genomic DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgta

SEQ ID NO: 23 - DNA, 145 bp, unidentified
  misc_feature 1..145: note = Adeno-associated virus
  source 1..145: mol_type = genomic DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 24 - DNA, 141 bp, unidentified
  misc_feature 1..141: note = Adeno-associated viurs
  source 1..141: mol_type = genomic DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 25 - DNA, 168 bp, unidentified
  misc_feature 1..168: note = Adeno-associated virus 2
  source 1..168: mol_type = genomic DNA
tacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 26 - DNA, 133 bp, unidentified
  misc_feature 1..133: note = Adeno-associated virus 2
  source 1..133: mol_type = genomic DNA
aggaacccctagtgatggagactccctctctgcgcgctcgctcgctcactgaggccgggcgaccaaaggtcgcccgacgcccgggctttgcccgggcggcctcagtgagcgagcgagcgcgcagagagggagt

SEQ ID NO: 27 - DNA, 124 bp, synthetic construct
  misc_feature 1..124: note = Made in Lab - vector filler sequence
  source 1..124: mol_type = other DNA
gcggcaattcagtcgataactataacggtcctaaggtagcgatttaaatacgcgctctcttaaggtagccccgggacgcgtcaattgactacaaaccgagtatctgcagagggccctgcgtatg

SEQ ID NO: 28 - DNA, 84 bp, synthetic construct
  misc_feature 1..84: note = Made in Lab - vector filler sequence
  source 1..84: mol_type = other DNA
cttctgaggcggaaagaaccagatcctctcttaaggtagcatcgagatttaaattagggataacagggtaatggcgcgggccgc

SEQ ID NO: 29 - DNA, 63 bp, synthetic construct
  misc_feature 1..63: note = Made in Lab - vector filler sequence
  source 1..63: mol_type = other DNA
gttacccaggctggagtgcagtggcacatttctgctcactgcaacctcctcctccctgggttc

SEQ ID NO: 30 - DNA, 573 bp, synthetic construct
  misc_feature 1..573: note = Made in lab - CAG promoter in part Human betaherpesvirus 5
  source 1..573: mol_type = other DNA
acttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgg

SEQ ID NO: 31 - DNA, 771 bp, synthetic construct
  misc_feature 1..771: note = Made in Lab - MHCK7 promoter
  source 1..771: mol_type = other DNA
acccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccag

SEQ ID NO: 32 - DNA, 544 bp, Homo sapiens
  source 1..544: mol_type = genomic DNA
ctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcag

SEQ ID NO: 33 - DNA, 502 bp, Homo sapiens
  source 1..502: mol_type = other DNA
ctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagt

SEQ ID NO: 34 - DNA, 5464 bp, Mus musculus
  source 1..5464: mol_type = genomic DNA
ggtaccggatcctgcaaggtcacacaagggtctccacccaccaggtgccctagtctcaatttcagtttccatgccttgttctcacaatgctggcctccccagagctaatttggactttgtttttatttcaaaagggcctgaatgaggagtagatcttgtgctacccagctctaagggtgcccgtgaagccctcagacctggagcctttgcaacagccctttaggtggaagcagaataaagcaattttccttaaagccaaaatcctgcctctagactcttcttctctgacctcggtccctgggctctagggtggggaggtggggcttggaagaagaaggtggggaagtggcaaaagccgatccctagggccctgtgaagttcggagccttccctgtacagcactggctcatagatcctcctccagccaaacatagcaagaagtgatacctcctttgtgacttccccaggcccagtacctgtcaggttgaaacaggatttagagaagcctctgaactcacctgaactctgaagctcatccaccaagcaagcacctaggtgccactgctagttagtatcctacgctgataatatgcagagctgggccacagaagtcctggggtgtaggaactgaccagtgacttttcagtcggcaaaggtatgaccccctcagcagatgtagtaatgtccccttagatcccatcccaggcaggtctctaagaggacatgggatgagagatgtagtcatgtggcattccaaacacagctatccacagtgtcccttgccccttccacttagccaggaggacagtaaccttagcctatctttcttcctccccatcctcccaggacacaccccctggtctgcagtattcatttcttccttcacgtcccctctgtgacttccatttgcaaggcttttgacctctgcagctgctggaagatagagtttggccctaggtgtggcaagccatctcaagagaaagcagacaacagggggaccagattttggaaggatcaggaactaaatcactggcgggcctgggggtagaaaaaagagtgagtgagtccgctccagctaagccaagctagtccccgagatactctgccacagctgggctgctcggggtagctttaggaatgtgggtctgaaagacaatgggattggaagacatctctttgagtctcccctcaaccccacctacagacacactcgtgtgtggccagactcctgttcaacagccctctgtgttctgaccactgagctaggcaaccagagcatgggccctgtgctgaggatgaagagttggttaccaatagcaaaaacagcaggggagggagaacagagaacgaaataaggaaggaagaaggaaaggccagtcaatcagatgcagtcagaagagatgggaagccaacacacagcttgagcagaggaaacagaaaagggagagattctgggcataaggaggccacagaaagaagagcccaggccccccaagtctcctctttataccctcatcccgtctcccaattaagcccactcttcttcctagatcagacctgagctgcagcgaagagacccgtagggaggatcacactggatgaaggagatgtgtggagaagtccagggaacctaagagccagagcctaaaagagcaagagataaaggtgcttcaaaggtggccaggctgtgcacacagagggtcgaggactggtggtagagcctcaagataaggatgatgctcagaatgggcggggggggggattctgggggggggagagagaaggtgagaaggagcctggaacagagaatctggaagcgctggaaacgataccataaagggaagaacccaggctacctttagatgtaaatcatgaaagacagggagaagggaagctggagagagtagaaggaccccggggcaagacattgaagcaaggacaagccaggttgagcgctccgtgaaatcagcctgctgaaggcagagccctggtatgagcaccagaacagcagaggctagggttaatgtcgagacagggaacagaaggtagacacaggaacagacagagacgggggagccaggtaacaaaggaatggtccttctcacctgtggccagagcgtccatctgtgtccacatactctagaatgttcatcagactgcagggctggcttgggaggcagctggaaagagtatgtgagagccaggggagacaagggggcctaggaaaggaagaagagggcaaaccaggccacacaagagggcagagcccagaactgagttaactccttccttgttgcatcttccataggaggcagtgggaactctgtgaccaccatcccccatgagcccccactacccataccaagtttggcctgagtggcattctaggttccctgaggacagagcctggcctttgtctcttggacctgacccaagctgacccaatgttctcagtaccttatcatgccctcaagagcttgagaaccaggcagtgacatattaggccatgggctaaccctggagcttgcacacaggagcctcaagtgacctccagggacacagctgcagacaggtggcctttatccccaaagagcaaccatttggcataggtggctgcaaatgggaatgcaaggttgaatcaggtcccttcaagaatactgcatgcaagacctaagacccctggagagaggggtatgctcctgcccccacccaccataaggggagtgaactatcctagggggctggcgaccttggggagacaccacattactgagagtgctgagcccagaaaaactgaccgccctgtgtcctgcccacctccacactctagagctatattgagaggtgacagtagatagggtgggagctggtagcagggagagtgttcctgggtgtgagggtgtaggggaaagccagagcaggggagtctggctttgtctcctgaacacaatgtctacttagttataacaggcatgacctgctaaagacccaacatctacgacctctgaaaagacagcagccctggaggacaggggttgtctctgagccttgggtgcttgatggtgccacaaaggagggcatgagtgtgagtataaggccccaggagcgttagagaagggcacttgggaaggggtcagtctgcagagcccctatccatggaatctggagcctggggccaactggtgtaaatctctgggcctgccaggcattcaaagcagcacctgcatcctctggcagcctggggaggcggaagggagcaaccccccacttataccctttctccctcagccccaggattaacacctctggccttcccccttcccacctcccatcaggagtggagggttgcagagggagggtaaaaacctacatgtccaaacatcatggtgcacgatatatggatcagtatgtgtagaggcaagaaaggaaatctgcaggcttaactgggttaatgtgtaaagtctgtgtgcatgtgtgtgtgtctgactgaaaacgggcatggctgtgcagctgttcagttctgtgcgtgaggttaccagactgcaggtttgtgtgtaaattgcccaaggcaaagtgggtgaatcccttccatggtttaaagagattggatgatggcctgcatctcaaggaccatggaaaatagaatggacactctatatgtgtctctaagctaaggtagcaaggtctttggaggacacctgtctagagatgtgggcaacagagactacagacagtatctgtacagagtaaggagagagaggagggggtgtagaattctcttactatcaaagggaaactgagtcgtgcacctgcaaagtggatgctctccctagacatcatgactttgtctctggggagccagcactgtggaacttcaggtctgagagagtaggaggctcccctcagcctgaagctatgcagatagccagggttgaaagggggaagggagagcctgggatgggagcttgtgtgttggaggcaggggacagatattaagcctggaagagaaggtgacccttacccagttgttcaactcacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtcctcctgtcacctccagagccaagggatcaaaggaggaggagccaggacaggagggaagtgggagggagggtcccagcagaggactccaaatttaggcagcaggcatatgggatgggatataaaggggctggagcactgagagctgtcagagatttctccaacccaggtaagagggagtttcgggtgggggctcttcacccacaccagacctctccccacctagaaggaaactgcctttcctggaagtggggttcaggccggtcagagatctgacagggtggccttccaccagcctgggaagttctcagtggcaggaggtttccacaagaaacactggatgccccttcccttacgctgtcttctccatcttcctcctggggatgctcctccccgtcttggtttatcttggctcttcgtcttcagcaagatttgccctgtgctgtccactccatctttctctactgtctccgtgccttgccttgccttcttgcgtgtccttcctttccacccatttctcacttcaccttttctccccttctcatttgtattcatccttccttccttccttccttccttccttccttccttccttccttcctttctcccttccttccttccttccttccttccttccttccttccttcctgtgtcagagtgctgagaatcacacctggggttcccacccttatgtaaacaatcttccagtgagccacagcttcagtgctgctgggtgctctcttaccttcctcaccccctggcttgtcctgttccatcctggtcaggatctctagattggtctcccagcctctgctactcctcttcctgcctgttcctctctctgtccagctgcgccactgtggtgcctcgttccagctgtggtccacattcttcaggattctctgaaaagttaaccaggtgagaatgtttcccctgtagacagcagatcacgattctcccggaagtcaggcttccagccctctctttctctgcccagctgcccggcactcttagcaaacctcaggcacccttaccccacatagacctctgacagagaagcaggcactttacatggagtcctggtgggagagccataggctacggtgtaaaagaggcagggaagtggtggtgtaggaaagtcaggacttcacatagaagcctagcccacaccagaaatgacagacagatccctcctatctcccccataagagtttgagtcgacccgcggccccgaattg

SEQ ID NO: 35 - DNA, 413 bp, Gallus gallus
  source 1..413: mol_type = genomic DNA
gggataaaagcagtctgggctttcacatgacagcatctggggctgcggcagagggtcgggtccgaagcgctgccttatcagcgtccccagccctgggaggtgacagctggctggcttgtgtcagcccctcgggcactcacgtatctccgtccgacgggtttaaaatagcaaaactctgaggccacacaatagcttgggcttatatgggctcctgtgggggaagggggagcacggagggggccggggccgctgctgccaaaatagcagctcacaagtgttgcattcctctctgggcgccgggcacattcctgctggctctgcccgccccggggtgggcgccggggggaccttaaagcctctgccccccaaggagcccttcccagacagccgccggcacccaccgctccgtggga

SEQ ID NO: 36 - DNA, 1090 bp, Homo sapiens
  source 1..1090: mol_type = genomic DNA
ctctcagccctggaagtccttgctcacagccgaggcgccgagagcgcttgctctgcccagatctgcgcgagtctggcgcccgcgctctgaacggcgtcgctgcccagcccccttccccgggaggtgggagcggccacccagggccccgtggctgcccttgtaaggaggcgaggcccgaggacacccgagacgcccggttataattaaccaggacacgtggcgaacccccctccaacacctgcccccgaacccccccatacccagcgcctcgggtctcggcctttgcggcagaggagacagcaaagcgccctctaaaaataactcctttcccggcgaccgagaccctccctgtcccccgcacagcggaaatctcccagtggcaccgagggggcgagggttaagtgggggggagggtgaccaccgcctcccacccttgccctgagtttgaatctctccaactcagccagcctcagtttcccctccactcagtccctaggaggaaggggcgcccaagcgcgggtttctggggttagactgccctccattgcaattggtccttctcccggcctctgcttcctccagctcacagggtatctgctcctcctggagccacaccttggttccccgaggtgccgctgggactcgggtaggggtgagggcccagggggcacagggggagccgagggccacaggaagggctggtggctgaaggagactcaggggccaggggacggtggcttctacgtgcttgggacgttcccagccaccgtcccatgttcccggcggggggccagctgtccccaccgccagcccaactcagcacttggtcagggtatcagcttggtgggggggcgtgagcccagcccctggggcggctcagcccatacaaggccatggggctgggcgcaaagcatgcctgggttcagggtgggtatggtgcgggagcagggaggtgagaggctcagctgccctccagaactcctccctggggacaacccctcccagccaatagcacagcctaggtccccctatataaggccacggctgctggcccttcctttgggtcagtgtcacctccaggatacagaca

SEQ ID NO: 37 - DNA, 253 bp, Homo sapiens
  source 1..253: mol_type = genomic DNA
gcccagcaccccaaggcggccaacgccaaaactctccctcctcctcttcctcaatctcgctctcgctctttttttttttcgcaaaaggaggggagagggggtaaaaaaatgctgcactgtgcggcgaagccggtgagtgagcggcgcggggccaatcagcgtgcgccgttccgaaagttgccttttatggctcgagcggccgcggcggcgccctataaaacccagcggcgcgacgcgccaccaccgccgagtc

SEQ ID NO: 38 - DNA, 281 bp, Gallus gallus
  source 1..281: mol_type = genomic DNA
ggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggga

SEQ ID NO: 39 - DNA, 220 bp, Human betaherpesvirus 5
  source 1..220: mol_type = genomic DNA
tggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccg

SEQ ID NO: 40 - DNA, 583 bp, Human betaherpesvirus 5
  source 1..583: mol_type = genomic DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgt

SEQ ID NO: 41 - DNA, 508 bp, Human betaherpesvirus 5
  source 1..508: mol_type = genomic DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 42 - DNA, 573 bp, synthetic construct; identical to SEQ ID NO: 30
  misc_feature 1..573: note = Made in lab - CAG promoter in part Human betaherpesvirus 5
  source 1..573: mol_type = other DNA

SEQ ID NO: 43 - DNA, 580 bp, synthetic construct
  misc_feature 1..580: note = Made in lab - CAG promoter in part Human betaherpesvirus 5
  source 1..580: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 44 - DNA, 455 bp, Homo sapiens
  source 1..455: mol_type = genomic DNA
caacctttggagctaagccagcaatggtagagggaagattctgcacgtcccttccaggcggcctccccgtcaccaccccccccaacccgccccgaccggagctgagagtaattcatacaaaaggactcgcccctgccttggggaatcccagggaccgtcgttaaactcccactaacgtagaacccagagatcgctgcgttcccgccccctcacccgcccgctctcgtcatcactgaggtggagaatagcatgcgtgaggctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgtt

SEQ ID NO: 45-47 - skipped (3 entries)

SEQ ID NO: 48 - DNA, 281 bp, Homo sapiens
  source 1..281: mol_type = genomic DNA
acttgtggacaaagtttgctctattccacctcctccaggccctccttgggtccatcaccccaggggtgctgggtccatcccacccccaggcccacacaggcttgcagtattgtgtgcggtatggtcagggcgtccgagagcaggtttcgcagtggaaggcaggcaggtgttggggaggcagttaccggggcaacgggaacagggcgttttggaggtggttgccatggggacctggatgctgacgaaggctcgcgaggctgtgagcagccacagtgccctgc

SEQ ID NO: 49 - skipped

SEQ ID NO: 50 - DNA, 293 bp, Human betaherpesvirus 5
  source 1..293: mol_type = genomic DNA
acttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattacca

SEQ ID NO: 51 - DNA, 953 bp, Homo sapiens
  source 1..953: mol_type = genomic DNA
cgcgtccgcccgcgagcacagagcctcgcctttgccgatccgccgcccgtccacacccgccgccaggtaagcccggccagccgaccggggcatgcggccgcggcccttcgcccgtgcagagccgccgtctgggccgcagcggggggcgcatggggcggaaccggaccgccgtggggggcgcgggagaagcccctgggcctccggagatgggggacaccccacgccagttcgcaggcgcgaggccgcgctcgggcgggcgcgctccgggggtgccgctctcggggcgggggcaaccggcggggtctttgtctgagccgggctcttgccaatggggatcgcacggtgggcgcggcgtagcccccgtcaggcccggtgggggctggggcgccatgcgcgtgcgcgctggtcctttgggcgctaactgcgtgcgcgctgggaattggcgctaattgcgcgtgcgcgctgggactcaatggcgctaatcgcgcgtgcgttctggggcccgggcgcttgcgccacttcctgcccgagccgctggcgcccgagggtgtggccgctgcgtgcgcgcgcgcgacccggtcgctgtttgaaccgggcggaggcggggctggcgcccggttgggagggggttggggcctggcttcctgccgcgcgccgcggggacgcctccgaccagtgtttgccttttatggtaataacgcggccggcccggcttcctttgtccccaatctgggcgcgcgccggcgccccctggcggcctaaggactcggcgcgccggaagtggccagggcggcagcggctgctcttggcggccccgaggtgactatagccttcttttgtgtcttgatagttcgccagcctctgctaaccatgttcatgccttcttctttttcctacagctcctgggcaacgtgctggttattgtgctgtctcatcattttggcaaagaattc

SEQ ID NO: 52 - DNA, 1068 bp, synthetic construct
  misc_feature 1..1068: note = Made in Lab - Chicken beta-actin exon/intron plus rabbit globin intron
  source 1..1068: mol_type = other DNA
gtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattc

SEQ ID NO: 53 - DNA, 149 bp, synthetic construct
  misc_feature 1..149: note = Made in Lab - chimeric intron sequence
  source 1..149: mol_type = other DNA
ggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgc

SEQ ID NO: 54 - DNA, 126 bp, Homo sapiens
  source 1..126: mol_type = genomic DNA
agtctgcggtgggcagcggaggagtcgtgtcgtgcctgagagcgcagctgtgctcctgggcaccgcgcagtccgcccccgcggctcctggccagaccacccctaggaccccctgccccaagtcgca

SEQ ID NO: 55 - DNA, 121 bp, Human betaherpesvirus 5
  source 1..121: mol_type = genomic DNA
tcagatcgcctggagaggccatccacgctgttttgacctccatagtggacaccgggaccgatccagcctccgcggccgggaacggtgcattggaacgcggattccccgtgccaagagtgac

SEQ ID NO: 56 - DNA, 512 bp, synthetic construct
  misc_feature 1..512: note = Made in Lab - adenovirus derived enhancer element
  source 1..512: mol_type = other DNA
ctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccag

SEQ ID NO: 57 - DNA, 956 bp, Homo sapiens
  source 1..956: mol_type = genomic DNA
ctttttcgcaacgggtttgccgccagaacacaggtaagtgccgtgtgtggttcccgcgggcctggcctctttacgggttatggcccttgcgtgccttgaattacttccacctggctccagtacgtgattcttgatcccgagctggagccaggggcgggccttgcgctttaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacgtgctgcgacgctttttttctggcaagatagtcttgtaaatgcgggccaggatctgcacactggtatttcggtttttgggcccgcggccggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctccagggggctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctggagcttttggagtacgtcgtctttaggttggggggaggggttttatgcgatggagtttccccacactgagtgggtggagactgaagttaggccagcttggcacttgatgtaattctccttggaatttggcctttttgagtttggatcttggttcattctcaagcctcagacagtggttcaaagtttttttcttccatttcag

SEQ ID NO: 58 - DNA, 939 bp, Homo sapiens
  source 1..939: mol_type = genomic DNA
gtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcag

SEQ ID NO: 59 - DNA, 83 bp, Homo sapiens
  source 1..83: mol_type = genomic DNA
tcagaagccccgggctcgtcagtcaaaccggttctctgtttgcactcggcagcacgggcaggcaagtggtccctaggttcggg

SEQ ID NO: 60 - DNA, 476 bp, Homo sapiens
  source 1..476: mol_type = genomic DNA
gtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 61 - DNA, 196 bp, unidentified
  misc_feature 1..196: note = simian virus 40
  source 1..196: mol_type = genomic DNA
tctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgc

SEQ ID NO: 62 - DNA, 589 bp, synthetic construct
  misc_feature 1..589: note = Made in Lab - mutated woodchuck hepatitis regulatory element
  source 1..589: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 63 - DNA, 588 bp, synthetic construct
  misc_feature 1..588: note = Made in Lab - mutated woodchuck hepatitis regulatory element
  source 1..588: mol_type = other DNA
tcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgca

SEQ ID NO: 64 - DNA, 755 bp, synthetic construct
  misc_feature 1..755: note = Made in Lab - mutated woodchuck hepatitis regulatory element
  source 1..755: mol_type = other DNA
ttcctgttaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcctcttcgccttcgccctcagacgagtcggatctccctttgggccgcctccccgcccatgtatctttttcacctgtgccttgtttttgcctgtgttccgcgtcctacttttcaagcctccaagctgtgccttgggcggctttggggcatggacatagatccctataaagaatttggttcatcttatcagttgttgaattttcttcctttggac

SEQ ID NO: 65 - DNA, 12 bp, synthetic construct
  misc_feature 1..12: note = CAAX motif
  source 1..12: mol_type = other DNA
tgtgtgataatg

SEQ ID NO: 66 - DNA, 810 bp, Homo sapiens
  source 1..810: mol_type = unassigned DNA
ctgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagtta

SEQ ID NO: 67 - DNA, 726 bp, Hepatitis B virus
  source 1..726: mol_type = unassigned DNA
ataacaggcctattgattggaaagtttgtcaacgaattgtgggtcttttggggtttgctgccccttttacgcaatgtggatatcctgctttaatgcctttatatgcatgtatacaagcaaaacaggcttttactttctcgccaacttacaaggcctttctcagtaaacagtatatgaccctttaccccgttgctcggcaacggcctggtctgtgccaagtgtttgctgacgcaacccccactggttggggcttggccataggccatcagcgcatgcgtggaacctttgtgtctcctctgccgatccatactgcggaactcctagccgcttgttttgctcgcagcaggtctggagcaaacctcatcgggaccgacaattctgtcgtactctcccgcaagtatacatcgtttccatggctgctaggctgtgctgccaactggatcctgcgcgggacgtcctttgtttacgtcccgtcggcgctgaatcccgcggacgacccctcccggggccgcttggggctctaccgcccgcttctccgtctgccgtaccgtccgaccacggggcgcacctctctttacgcggactccccgtctgtgccttctcatctgccggaccgtgtgcacttcgcttcacctctgcacgtcgcatggaggccaccgtgaacgcccaccggaacctgcccaaggtcttgcataagaggactcttggactttcagcaatgtcatc

SEQ ID NO: 68 - DNA, 755 bp, synthetic construct
  misc_feature 1..755: note = Made in Lab - HepB derived enhancer element
  source 1..755: mol_type = other DNA
ttcctgtaaacaggcctattgattggaaagtttgtcaacgaattgtgggtcttttggggtttgctgccccttttacgcaatgtggatatcctgctttaatgcctttatatgcatgtatacaagcaaaacaggcttttactttctcgccaacttacaaggcctttctcagtaaacagtatatgaccctttaccccgttgctcggcaacggcctggtctgtgccaagtgtttgctgacgcaacccccactggttggggcttggccataggccatcagcgcatgcgtggaacctttgtgtctcctctgccgatccatactgcggaactcctagccgcttgttttgctcgcagctggactggagcaaacctcatcgggaccgacaattctgtcgtactctcccgcaagcactcaccgtttccgcggctgctcgcctgtgttgccacctggattctgcgcgggacgtccttctgctacgtcccttcggccctcaatccagcggaccttccttcccgcggcctgctgccggctctgcggcctcttccgcctcttcgccttcgccctcagacgagtcggatctccctttgggccgcctccccgcccatgtatctttttcacctgtgccttgtttttgcctgtgttccgcgtcctacttttcaagcctccaagctgtgccttgggcggctttggggcatggacatagatccctataaagaatttggttcatcttatcagttgttgaattttcttcctttggac

SEQ ID NO: 69 - DNA, 94 bp, Homo sapiens
  source 1..94: mol_type = genomic DNA
gctggagcctcggtagccgttcctcctgcccgctgggcctcccaacgggccctcctcccctccttgcaccggcccttcctggtctttgaataaa

SEQ ID NO: 70 - DNA, 596 bp, Woodchuck hepatitis virus
  source 1..596: mol_type = other DNA
attcgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagggcc

SEQ ID NO: 71 - DNA, 387 bp, Oryctolagus cuniculus
  source 1..387: mol_type = genomic DNA
tggctaataaaggaaatttattttcattgcaatagtgtgttggaattttttgtgtctctcactcggaagaacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatc

SEQ ID NO: 72 - DNA, 251 bp, Bos taurus
  source 1..251: mol_type = genomic DNA
ttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaatacaatagcaggcatgctggggatgcggtgggctctatgggtacccaggtgctgaagaattgacccggttcctcctggg

SEQ ID NO: 73 - DNA, 251 bp, Bos taurus
  source 1..251: mol_type = genomic DNA
ttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctggg

SEQ ID NO: 74 - DNA, 225 bp, Bos taurus
  source 1..225: mol_type = genomic DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 75 - DNA, 202 bp, Homo sapiens
  source 1..202: mol_type = genomic DNA
ctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgc

SEQ ID NO: 76 - protein, 735 aa, unidentified [sequence omitted]

SEQ ID NO: 77 - protein, 736 aa, unidentified
  REGION 1..736: note = Adeno-associated virus 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 78 - protein, 736 aa, unidentified
  REGION 1..736: note = Adeno-associated virus 6
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 79 - protein, 738 aa, unidentified
  REGION 1..738: note = Non-human primate Adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 80 - DNA, 2217 bp, unidentified
  misc_feature 1..2217: note = Non-human primate adeno-associated virus
  source 1..2217: mol_type = genomic DNA
atggctgccgatggttatcttccagattggctcgaggacaacctctctgagggcattcgcgagtggtgggacctgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctccaagcgggtgacaatccgtacctgcggtataatcacgccgacgccgagtttcaggagcgtctgcaagaagatacgtcttttgggggcaacctcgggcgcgcagtcttccaggccaaaaagcgggttctcgaacctctgggcctggttgaatcgccggttaagacggctcctggaaagaagagaccggtagagccatcaccccagcgctctccagactcctctacgggcatcggcaagaaaggccagcagcccgcaaaaaagagactcaattttgggcagactggcgactcagagtcagtccccgaccctcaaccaatcggagaaccaccagcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgcacctgggccctgcccacctacaacaaccacctctacaagcaaatctccaacgggacctcgggaggaagcaccaacgacaacacctacttcggctacagcaccccctgggggtattttgacttcaacagattccactgccacttttcaccacgtgactggcagcgactcatcaacaacaactggggattccggcccaagaggctcaacttcaagctcttcaacatccaagtcaaggaggtcacgcagaatgaaggcaccaagaccatcgccaataaccttaccagcacgattcaggtctttacggactcggaataccagctcccgtacgtgctcggctcggcgcaccagggctgcctgcctccgttcccggcggacgtcttcatgattcctcagtacgggtacctgactctgaacaatggcagtcaggctgtgggccggtcgtccttctactgcctggagtactttccttctcaaatgctgagaacgggcaacaactttgaattcagctacaacttcgaggacgtgcccttccacagcagctacgcgcacagccagagcctggaccggctgatgaaccctctcatcgaccagtacttgtactacctgtcccggactcaaagcacgggcggtactgcaggaactcagcagttgctattttctcaggccgggcctaacaacatgtcggctcaggccaagaactggctacccggtccctgctaccggcagcaacgcgtctccacgacactgtcgcagaacaacaacagcaactttgcctggacgggtgccaccaagtatcatctgaatggcagagactctctggtgaatcctggcgttgccatggctacccacaaggacgacgaagagcgattttttccatccagcggagtcttaatgtttgggaaacagggagctggaaaagacaacgtggactatagcagcgtgatgctaaccagcgaggaagaaataaagaccaccaacccagtggccacagaacagtacggcgtggtggccgataacctgcaacagcaaaacgccgctcctattgtaggggccgtcaatagtcaaggagccttacctggcatggtgtggcagaaccgggacgtgtacctgcagggtcccatctgggccaagattcctcatacggacggcaactttcatccctcgccgctgatgggaggctttggactgaagcatccgcctcctcagatcctgattaaaaacacacctgttcccgcggatcctccgaccaccttcaatcaggccaagctggcttctttcatcacgcagtacagtaccggccaggtcagcgtggagatcgagtgggagctgcagaaggagaacagcaaacgctggaacccagagattcagtacacttccaactactacaaatctacaaatgtggactttgctgtcaatactgagggtacttattccgagcctcgccccattggcacccgttacctcacccgtaatctgtaa

SEQ ID NO: 81 - protein, 535 aa, unidentified
  REGION 1..535: note = Non-human primate adeno-associated virus
MAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 82 - protein, 398 aa, unidentified
  REGION 1..398: note = Non-human primate adeno-associated virus
STIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 83 - protein, 332 aa, unidentified
  REGION 1..332: note = Non-human primate adeno-associated virus
RTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 84 - protein, 743 aa, synthetic construct
  REGION 1..743: note = Synthetic construct - AAV9 variant
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQTLAVPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 85 - protein, 7 aa, synthetic construct
  REGION 1..7: note = Peptide insert
TLAVPFK

SEQ ID NO: 86 - protein, 7 aa, synthetic construct
  REGION 1..7: note = Peptide insert
KFPVALT

SEQ ID NO: 87 - DNA, 2514 bp, synthetic construct
  misc_feature 1..2514: note = Made in Lab - codon optimized PKP2a polynucleotide
  source 1..2514: mol_type = other DNA
atggctgcacctggtgcgcctgccgaatatggttatataaggacagttttgggtcaacagatacttggacaacttgattcctcttctttggcgctcccttcagaagcaaagttgaaattggcaggttcatcaggtagaggaggacaaactgtgaaatctctgcggattcaggaacaagtacaacagactttggctagaaagggaagatcttccgttggcaatggaaatttgcatagaacgagcagcgtacctgaatacgtatacaatctccatcttgttgaaaacgattttgtaggaggtcggtctccagttccgaaaacatatgatatgcttaaagcaggtactacagctacatacgaaggtagatggggaagaggaactgcacaatattcatctcaaaaatctgttgaagaaaggtcattgcgacatcctctcagaaggctcgaaatttccccggattcatcacctgaaagggctcattatacacacagtgattatcaatatagccaacgatctcaagcaggtcatacacttcatcatcaggaaagtaggcgggcggcattgcttgtaccccctcgatatgccaggtcagaaattgtaggagtttccagagcaggtactactagtcgacaaagacactttgacacatatcataggcaatatcaacacggtagtgtctcagacactgtttttgattcaataccagcaaatccggcattgctgacgtatcctcgacctggtacgtcaaggagcatgggaaatcttctggagaaagaaaattatttgactgctgggcttacagttggacaggtacggccattggttcctcttcaaccagttactcagaatcgagcttccagaagctcttggcatcaatcaagctttcattccacaaggacactgagagaagcaggaccttctgttgccgttgattccagtgggcggagagcccaccttacagtaggtcaagcagctgccggaggatcaggtaatttgcttacagaaaggagtacttttacagatagccaactcgggaatgcggatatggaaatgacattggagagagctgtatcaatgctggaagcggatcatatgcttccttctagaatttccgctgcggcaacttttatacagcacgaatgttttcaaaaatccgaagcgcgaaagcgggttaatcagctgagaggtattctgaaactgttgcaacttttgaaagtgcaaaatgaagatgttcaaagagctgtttgtggagctcttagaaatcttgtttttgaagataatgataataaattggaagttgctgaacttaatggtgtacctagattgcttcaagttttgaaacaaacaagagatttggaaacaaaaaaacaaataacaggacttttgtggaatctttcttctaatgataaacttaaaaatttgatgataacagaagctttgttgacacttacagaaaatattattatacctttttctggatggcctgaaggggattatccaaaagctaatgggttgctggattttgatattttttataatgtgacaggatgtttgaggaatatgtcctcagctggtgctgacggtcggaaagcaatgaggcgatgtgacggtttgatcgattcccttgttcattacgttaggggaacaatagctgattatcaaccagatgataaagcaacagaaaattgtgtttgtattcttcataatctgtcttatcaattggaagccgaacttcctgaaaaatactctcaaaatatctatattcagaatcggaacatacaaactgataataacaaatctataggatgttttggatcaagaagcagaaaagttaaggaacaatatcaagacgttcctatgcccgaagaaaaatcaaatccaaagggtgtagaatggctgtggcattctattgttattagaatgtatttgtcactcatagcaaaatctgtacgaaattatacacaagaagcttctttgggagcccttcagaatcttacagcaggaagtggtcctatgcctacaagtgtagcgcaaacagttgtacagaaagaatctggattgcaacatactagaaagatgctgcatgtaggagatccttctgttaaaaagacagctataagcctcctgaggaacttgagtcgcaacttgtctctccagaatgagattgctaaggaaacgcttcctgaccttgtatctattatacctgacacggtgccttccacagatcttttgattgaaacaactgcatctgcttgttatacattgaataatatcattcaaaatagttatcagaacgcaagagatcttctgaatacaggaggtattcaaaaaataatggctatttcagcaggtgatgcatatgcttctaataaagcgtctaaagctgcatctgttcttttgtattctctttgggcacatacagaacttcaccatgcctacaagaaagctcaattcaagaaaacagattttgttaatagtcgcactgcaaaagcgtatcattcccttaaggattaa

SEQ ID NO: 88 - DNA, 2646 bp, synthetic construct
  misc_feature 1..2646: note = Made in Lab - codon optimized PKP2b polynucleotide
  source 1..2646: mol_type = other DNA
atggccgcacctggagctccagctgaatatggatatatcaggactgtcttgggtcagcagattctgggccaactggattcttcaagtctggccctcccgagtgaggcaaaactgaaactcgcaggatctagtggaaggggcggacagacagtgaagtcactcaggattcaggagcaggtccaacagacactggcaagaaaagggaggtccagcgtcggcaatggtaacttgcatagaacctcttcagtgcctgagtacgtgtacaacctgcacctggtggaaaacgattttgtgggcggcagatccccggtccctaaaacttatgacatgctgaaagctggcacgacagctacatatgaaggtagatggggccggggcaccgcacaatatagttctcagaagtccgtggaagaaagatctcttaggcacccacttcggcgactggagataagtccagacagcagccccgaaagggcgcattatacacattctgactaccaatacagccaaagatctcaggctggccacactctgcatcatcaggaaagtagaagggccgcccttctcgtccccccaagatacgcacggtcagaaatcgtgggagtgagcagagcaggtaccacctctcgacagaggcatttcgacacataccaccgacaataccaacatggatcagtgtcagatactgtttttgactccataccagcaaatccagcactcctgacatatcctagaccggggactagtcgctccatggggaatctgctcgaaaaagaaaactatctcaccgcaggtctgactgtggggcaggtaaggccgttggtgccccttcagcctgtaacgcagaatagggcttccaggtcatcctggcaccagagtagcttccactccacaaggactctgagagaggcaggccctagtgttgcagttgatagttccggtcgcagggcccatcttacagttggtcaagccgctgcagggggtagcggtaatctgctgaccgaaagaagcacattcacggattcccagctcggaaatgcagacatggagatgacacttgaacgagccgtgagcatgctggaagctgaccatatgctcccttctagaatctccgctgcggccactttcatccagcacgagtgctttcagaaatccgaagctaggaaacgcgtgaaccagctgaggggcatcctgaagctgctgcagctgcttaaggtgcagaatgaggatgtccaacgcgcagtgtgcggtgctctgaggaacctcgtgtttgaagataacgataataagttggaagtcgcagaacttaacggggtacctcgactgctccaggtgttgaaacagacacgggacctggaaacgaaaaaacaaattaccgaccacactgtgaatctcagaagtcggaatgggtggcctggggcagtagctcatgcctgtaacccttctacattgggtggccaaggcgggaggattaccagatcaggggtacgggatcaacctgatcagcacgggctcctgtggaacttgtcctctaatgacaaactgaaaaacctgatgataaccgaggctctgctgaccctgacagaaaacattatcatccccttctccggatggccagaaggagactatcctaaagctaacggcttgctggacttcgatatcttttataatgtcacagggtgccttagaaatatgagtagcgccggtgccgatggaaggaaggcaatgcgccggtgtgatggtcttatcgattccctggtacattacgtgaggggcaccattgccgactaccaaccagacgataaagcaaccgagaattgcgtgtgcatcctgcacaatttgagctaccaactcgaagctgaactgcctgagaaatattcccaaaatatttatatccagaatcggaatatccagaccgataataacaagagcataggatgctttggttctagaagccgcaaggtcaaggaacagtaccaggatgtacctatgccagaggaaaaatcaaaccccaaaggagtggaatggctgtggcattccatcgtgatcaggatgtacctgagtctgattgctaagagcgtgaggaactatacacaggaggcttcccttggcgcactgcaaaatctgaccgccggatctgggccaatgccaacaagcgttgctcaaacagtagtgcaaaaggaatctggccttcagcacactaggaaaatgcttcatgtgggagatccttctgtgaagaagacggctattagcctgctccgcaacttgagcagaaacctttctttgcagaatgagattgcgaaagagacgctccctgacctggtgtccattataccagacacggtcccttccacagatcttctgattgaaacgaccgcttccgcatgttacaccctgaataatattattcaaaactcataccaaaatgctagagacctgctgaacactggggggatccagaagattatggcaatttcagccggcgatgcctatgctagtaataaagcctccaaggccgccagtgtattgctgtatagtctgtgggcacatacagaactgcaccacgcatataagaaggcccagtttaaaaaaaccgatttcgtaaactctcggactgcaaaagcctaccattcactgaaagattga

SEQ ID NO: 89 - DNA, 4650 bp, synthetic construct
  misc_feature 1..4650: note = Made in Lab - polynucleotide sequence of vector genome
  source 1..4650: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctggagacgcgtacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcgccaccatggcagcccccggcgccccagctgagtacggctacatccggaccgtcctgggccagcagatcctgggacaactggacagctccagcctggcgctgccctccgaggccaagctgaagctggcggggagcagcggccgcggcggccagacagtcaagagcctgcggatccaggagcaggtgcagcagaccctcgcccggaagggccgcagctccgtgggcaacggaaatcttcaccgaaccagcagtgttcctgagtatgtctacaacctacacttggttgaaaatgattttgttggaggccgttcccctgttcctaaaacctatgacatgctaaaggctggcacaactgccacttatgaaggtcgctggggaagaggaacagcacagtacagctcccagaagtccgtggaagaaaggtccttgaggcatcctctgaggagactggagatttctcctgacagcagcccggagagggctcactacacgcacagcgattaccagtacagccagagaagccaggctgggcacaccctgcaccaccaagaaagcaggcgggccgccctcctagtgccaccgagatatgctcgttccgagatcgtgggggtcagccgtgctggcaccacaagcaggcagcgccactttgacacataccacagacagtaccagcatggctctgttagcgacaccgtttttgacagcatccctgccaacccggccctgctcacgtaccccaggccagggaccagccgcagcatgggcaacctcttggagaaggagaactacctgacggcagggctcactgtcgggcaggtcaggccgctggtgcccctgcagcccgtcactcagaacagggcttccaggtcctcctggcatcagagctccttccacagcacccgcacgctgagggaagctgggcccagtgtcgccgtggattccagcgggaggagagcgcacttgactgtcggccaggcggccgcagggggaagtgggaatctgctcactgagagaagcactttcactgactcccagctggggaatgcagacatggagatgactctggagcgagcagtgagtatgctcgaggcagaccacatgctgccatccaggatttctgctgcagctactttcatacagcacgagtgcttccagaaatctgaagctcggaagagggttaaccagcttcgtggcatcctcaagcttctgcagctcctaaaagttcagaatgaagacgttcagcgagctgtgtgtggggccttgagaaacttagtatttgaagacaatgacaacaaattggaggtggctgaactaaatggggtacctcggctgctccaggtgctgaagcaaaccagagacttggagactaaaaaacaaataacaggtttgctgtggaatttgtcatctaatgacaaactcaagaatctcatgataacagaagcattgcttacgctgacggagaatatcatcatccccttttctgggtggcctgaaggagactacccaaaagcaaatggtttgctcgattttgacatattctacaacgtcactggatgcctaagaaacatgagttctgctggcgctgatgggagaaaagcgatgagaagatgtgacggactcattgactcactggtccattatgtcagaggaaccattgcagattaccagccagatgacaaggccacggagaattgtgtgtgcattcttcataacctctcctaccagctggaggcagagctcccagagaaatattcccagaatatctatattcaaaaccggaatatccagactgacaacaacaaaagtattggatgttttggcagtcgaagcaggaaagtaaaagagcaataccaggacgtgccgatgccggaggaaaagagcaaccccaagggcgtggagtggctgtggcattccattgttataaggatgtatctgtccttgatcgccaaaagtgtccgcaactacacacaagaagcatccttaggagctctgcagaacctcacggccggaagtggaccaatgccgacatcagtggctcagacagttgtccagaaggaaagtggcctgcagcacacccgaaagatgctgcatgttggtgacccaagtgtgaaaaagacagccatctcgctgctgaggaatctgtcccggaatctttctctgcagaatgaaattgccaaagaaactctccctgatttggtttccatcattcctgacacagtcccgagtactgaccttctcattgaaactacagcctctgcctgttacacattgaacaacataatccaaaacagttaccagaatgcacgcgaccttctaaacaccgggggcatccagaaaattatggccattagtgcaggcgatgcctatgcctccaacaaagcaagtaaagctgcttccgtccttctgtattctctgtgggcacacacggaactgcatcatgcctacaagaaggctcagtttaagaagacagattttgtcaacagccggactgccaaagcctaccactcccttaaagactgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcgttacccaggctggagtgcagtggcacatttctgctcactgcaacctcctcctccctgggttctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 90 - DNA, 4274 bp, synthetic construct
  misc_feature 1..4274: note = Made in Lab - polynucleotide sequence of vector genome
  source 1..4274: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctggagacgcgtctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcaggccaccatggcagcccccggcgccccagctgagtacggctacatccggaccgtcctgggccagcagatcctgggacaactggacagctccagcctggcgctgccctccgaggccaagctgaagctggcggggagcagcggccgcggcggccagacagtcaagagcctgcggatccaggagcaggtgcagcagaccctcgcccggaagggccgcagctccgtgggcaacggaaatcttcaccgaaccagcagtgttcctgagtatgtctacaacctacacttggttgaaaatgattttgttggaggccgttcccctgttcctaaaacctatgacatgctaaaggctggcacaactgccacttatgaaggtcgctggggaagaggaacagcacagtacagctcccagaagtccgtggaagaaaggtccttgaggcatcctctgaggagactggagatttctcctgacagcagcccggagagggctcactacacgcacagcgattaccagtacagccagagaagccaggctgggcacaccctgcaccaccaagaaagcaggcgggccgccctcctagtgccaccgagatatgctcgttccgagatcgtgggggtcagccgtgctggcaccacaagcaggcagcgccactttgacacataccacagacagtaccagcatggctctgttagcgacaccgtttttgacagcatccctgccaacccggccctgctcacgtaccccaggccagggaccagccgcagcatgggcaacctcttggagaaggagaactacctgacggcagggctcactgtcgggcaggtcaggccgctggtgcccctgcagcccgtcactcagaacagggcttccaggtcctcctggcatcagagctccttccacagcacccgcacgctgagggaagctgggcccagtgtcgccgtggattccagcgggaggagagcgcacttgactgtcggccaggcggccgcagggggaagtgggaatctgctcactgagagaagcactttcactgactcccagctggggaatgcagacatggagatgactctggagcgagcagtgagtatgctcgaggcagaccacatgctgccatccaggatttctgctgcagctactttcatacagcacgagtgcttccagaaatctgaagctcggaagagggttaaccagcttcgtggcatcctcaagcttctgcagctcctaaaagttcagaatgaagacgttcagcgagctgtgtgtggggccttgagaaacttagtatttgaagacaatgacaacaaattggaggtggctgaactaaatggggtacctcggctgctccaggtgctgaagcaaaccagagacttggagactaaaaaacaaataacaggtttgctgtggaatttgtcatctaatgacaaactcaagaatctcatgataacagaagcattgcttacgctgacggagaatatcatcatccccttttctgggtggcctgaaggagactacccaaaagcaaatggtttgctcgattttgacatattctacaacgtcactggatgcctaagaaacatgagttctgctggcgctgatgggagaaaagcgatgagaagatgtgacggactcattgactcactggtccattatgtcagaggaaccattgcagattaccagccagatgacaaggccacggagaattgtgtgtgcattcttcataacctctcctaccagctggaggcagagctcccagagaaatattcccagaatatctatattcaaaaccggaatatccagactgacaacaacaaaagtattggatgttttggcagtcgaagcaggaaagtaaaagagcaataccaggacgtgccgatgccggaggaaaagagcaaccccaagggcgtggagtggctgtggcattccattgttataaggatgtatctgtccttgatcgccaaaagtgtccgcaactacacacaagaagcatccttaggagctctgcagaacctcacggccggaagtggaccaatgccgacatcagtggctcagacagttgtccagaaggaaagtggcctgcagcacacccgaaagatgctgcatgttggtgacccaagtgtgaaaaagacagccatctcgctgctgaggaatctgtcccggaatctttctctgcagaatgaaattgccaaagaaactctccctgatttggtttccatcattcctgacacagtcccgagtactgaccttctcattgaaactacagcctctgcctgttacacattgaacaacataatccaaaacagttaccagaatgcacgcgaccttctaaacaccgggggcatccagaaaattatggccattagtgcaggcgatgcctatgcctccaacaaagcaagtaaagctgcttccgtccttctgtattctctgtgggcacacacggaactgcatcatgcctacaagaaggctcagtttaagaagacagattttgtcaacagccggactgccaaagcctaccactcccttaaagactgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcgttacccaggctggagtgcagtggcacatttctgctcactgcaacctcctcctccctgggttctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 91 - DNA, 4782 bp, synthetic construct
  misc_feature 1..4782: note = Made in Lab - polynucleotide sequence of vector genome
  source 1..4782: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctggagacgcgtacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcgccaccatggcagcccccggcgccccagctgagtacggctacatccggaccgtcctgggccagcagatcctgggacaactggacagctccagcctggcgctgccctccgaggccaagctgaagctggcggggagcagcggccgcggcggccagacagtcaagagcctgcggatccaggagcaggtgcagcagaccctcgcccggaagggccgcagctccgtgggcaacggaaatcttcaccgaaccagcagtgttcctgagtatgtctacaacctacacttggttgaaaatgattttgttggaggccgttcccctgttcctaaaacctatgacatgctaaaggctggcacaactgccacttatgaaggtcgctggggaagaggaacagcacagtacagctcccagaagtccgtggaagaaaggtccttgaggcatcctctgaggagactggagatttctcctgacagcagcccggagagggctcactacacgcacagcgattaccagtacagccagagaagccaggctgggcacaccctgcaccaccaagaaagcaggcgggccgccctcctagtgccaccgagatatgctcgttccgagatcgtgggggtcagccgtgctggcaccacaagcaggcagcgccactttgacacataccacagacagtaccagcatggctctgttagcgacaccgtttttgacagcatccctgccaacccggccctgctcacgtaccccaggccagggaccagccgcagcatgggcaacctcttggagaaggagaactacctgacggcagggctcactgtcgggcaggtcaggccgctggtgcccctgcagcccgtcactcagaacagggcttccaggtcctcctggcatcagagctccttccacagcacccgcacgctgagggaagctgggcccagtgtcgccgtggattccagcgggaggagagcgcacttgactgtcggccaggcggccgcagggggaagtgggaatctgctcactgagagaagcactttcactgactcccagctggggaatgcagacatggagatgactctggagcgagcagtgagtatgctcgaggcagaccacatgctgccatccaggatttctgctgcagctactttcatacagcacgagtgcttccagaaatctgaagctcggaagagggttaaccagcttcgtggcatcctcaagcttctgcagctcctaaaagttcagaatgaagacgttcagcgagctgtgtgtggggccttgagaaacttagtatttgaagacaatgacaacaaattggaggtggctgaactaaatggggtacctcggctgctccaggtgctgaagcaaaccagagacttggagactaaaaaacaaataacagaccatacagtcaatttaagaagtaggaatggctggccgggcgcggtggctcacgcctgtaatcccagcactttgggaggccaaggcgggcggatcacgaggtcaggagttcgagaccagcctgaccaacatggtttgctgtggaatttgtcatctaatgacaaactcaagaatctcatgataacagaagcattgcttacgctgacggagaatatcatcatccccttttctgggtggcctgaaggagactacccaaaagcaaatggtttgctcgattttgacatattctacaacgtcactggatgcctaagaaacatgagttctgctggcgctgatgggagaaaagcgatgagaagatgtgacggactcattgactcactggtccattatgtcagaggaaccattgcagattaccagccagatgacaaggccacggagaattgtgtgtgcattcttcataacctctcctaccagctggaggcagagctcccagagaaatattcccagaatatctatattcaaaaccggaatatccagactgacaacaacaaaagtattggatgttttggcagtcgaagcaggaaagtaaaagagcaataccaggacgtgccgatgccggaggaaaagagcaaccccaagggcgtggagtggctgtggcattccattgttataaggatgtatctgtccttgatcgccaaaagtgtccgcaactacacacaagaagcatccttaggagctctgcagaacctcacggccggaagtggaccaatgccgacatcagtggctcagacagttgtccagaaggaaagtggcctgcagcacacccgaaagatgctgcatgttggtgacccaagtgtgaaaaagacagccatctcgctgctgaggaatctgtcccggaatctttctctgcagaatgaaattgccaaagaaactctccctgatttggtttccatcattcctgacacagtcccgagtactgaccttctcattgaaactacagcctctgcctgttacacattgaacaacataatccaaaacagttaccagaatgcacgcgaccttctaaacaccgggggcatccagaaaattatggccattagtgcaggcgatgcctatgcctccaacaaagcaagtaaagctgcttccgtccttctgtattctctgtgggcacacacggaactgcatcatgcctacaagaaggctcagtttaagaagacagattttgtcaacagccggactgccaaagcctaccactcccttaaagactgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcgttacccaggctggagtgcagtggcacatttctgctcactgcaacctcctcctccctgggttctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 92 - DNA, 4406 bp, synthetic construct
  misc_feature 1..4406: note = Made in Lab - polynucleotide sequence of vector genome
  source 1..4406: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctggagacgcgtctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcaggccaccatggcagcccccggcgccccagctgagtacggctacatccggaccgtcctgggccagcagatcctgggacaactggacagctccagcctggcgctgccctccgaggccaagctgaagctggcggggagcagcggccgcggcggccagacagtcaagagcctgcggatccaggagcaggtgcagcagaccctcgcccggaagggccgcagctccgtgggcaacggaaatcttcaccgaaccagcagtgttcctgagtatgtctacaacctacacttggttgaaaatgattttgttggaggccgttcccctgttcctaaaacctatgacatgctaaaggctggcacaactgccacttatgaaggtcgctggggaagaggaacagcacagtacagctcccagaagtccgtggaagaaaggtccttgaggcatcctctgaggagactggagatttctcctgacagcagcccggagagggctcactacacgcacagcgattaccagtacagccagagaagccaggctgggcacaccctgcaccaccaagaaagcaggcgggccgccctcctagtgccaccgagatatgctcgttccgagatcgtgggggtcagccgtgctggcaccacaagcaggcagcgccactttgacacataccacagacagtaccagcatggctctgttagcgacaccgtttttgacagcatccctgccaacccggccctgctcacgtaccccaggccagggaccagccgcagcatgggcaacctcttggagaaggagaactacctgacggcagggctcactgtcgggcaggtcaggccgctggtgcccctgcagcccgtcactcagaacagggcttccaggtcctcctggcatcagagctccttccacagcacccgcacgctgagggaagctgggcccagtgtcgccgtggattccagcgggaggagagcgcacttgactgtcggccaggcggccgcagggggaagtgggaatctgctcactgagagaagcactttcactgactcccagctggggaatgcagacatggagatgactctggagcgagcagtgagtatgctcgaggcagaccacatgctgccatccaggatttctgctgcagctactttcatacagcacgagtgcttccagaaatctgaagctcggaagagggttaaccagcttcgtggcatcctcaagcttctgcagctcctaaaagttcagaatgaagacgttcagcgagctgtgtgtggggccttgagaaacttagtatttgaagacaatgacaacaaattggaggtggctgaactaaatggggtacctcggctgctccaggtgctgaagcaaaccagagacttggagactaaaaaacaaataacagaccatacagtcaatttaagaagtaggaatggctggccgggcgcggtggctcacgcctgtaatcccagcactttgggaggccaaggcgggcggatcacgaggtcaggagttcgagaccagcctgaccaacatggtttgctgtggaatttgtcatctaatgacaaactcaagaatctcatgataacagaagcattgcttacgctgacggagaatatcatcatccccttttctgggtggcctgaaggagactacccaaaagcaaatggtttgctcgattttgacatattctacaacgtcactggatgcctaagaaacatgagttctgctggcgctgatgggagaaaagcgatgagaagatgtgacggactcattgactcactggtccattatgtcagaggaaccattgcagattaccagccagatgacaaggccacggagaattgtgtgtgcattcttcataacctctcctaccagctggaggcagagctcccagagaaatattcccagaatatctatattcaaaaccggaatatccagactgacaacaacaaaagtattggatgttttggcagtcgaagcaggaaagtaaaagagcaataccaggacgtgccgatgccggaggaaaagagcaaccccaagggcgtggagtggctgtggcattccattgttataaggatgtatctgtccttgatcgccaaaagtgtccgcaactacacacaagaagcatccttaggagctctgcagaacctcacggccggaagtggaccaatgccgacatcagtggctcagacagttgtccagaaggaaagtggcctgcagcacacccgaaagatgctgcatgttggtgacccaagtgtgaaaaagacagccatctcgctgctgaggaatctgtcccggaatctttctctgcagaatgaaattgccaaagaaactctccctgatttggtttccatcattcctgacacagtcccgagtactgaccttctcattgaaactacagcctctgcctgttacacattgaacaacataatccaaaacagttaccagaatgcacgcgaccttctaaacaccgggggcatccagaaaattatggccattagtgcaggcgatgcctatgcctccaacaaagcaagtaaagctgcttccgtccttctgtattctctgtgggcacacacggaactgcatcatgcctacaagaaggctcagtttaagaagacagattttgtcaacagccggactgccaaagcctaccactcccttaaagactgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcgttacccaggctggagtgcagtggcacatttctgctcactgcaacctcctcctccctgggttctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 93 - DNA, 4237 bp, synthetic construct
  misc_feature 1..4237: note = Made in Lab - expression cassette polynucleotide
  source 1..4237: mol_type = other DNA
acccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcgccaccatggcagcccccggcgccccagctgagtacggctacatccggaccgtcctgggccagcagatcctgggacaactggacagctccagcctggcgctgccctccgaggccaagctgaagctggcggggagcagcggccgcggcggccagacagtcaagagcctgcggatccaggagcaggtgcagcagaccctcgcccggaagggccgcagctccgtgggcaacggaaatcttcaccgaaccagcagtgttcctgagtatgtctacaacctacacttggttgaaaatgattttgttggaggccgttcccctgttcctaaaacctatgacatgctaaaggctggcacaactgccacttatgaaggtcgctggggaagaggaacagcacagtacagctcccagaagtccgtggaagaaaggtccttgaggcatcctctgaggagactggagatttctcctgacagcagcccggagagggctcactacacgcacagcgattaccagtacagccagagaagccaggctgggcacaccctgcaccaccaagaaagcaggcgggccgccctcctagtgccaccgagatatgctcgttccgagatcgtgggggtcagccgtgctggcaccacaagcaggcagcgccactttgacacataccacagacagtaccagcatggctctgttagcgacaccgtttttgacagcatccctgccaacccggccctgctcacgtaccccaggccagggaccagccgcagcatgggcaacctcttggagaaggagaactacctgacggcagggctcactgtcgggcaggtcaggccgctggtgcccctgcagcccgtcactcagaacagggcttccaggtcctcctggcatcagagctccttccacagcacccgcacgctgagggaagctgggcccagtgtcgccgtggattccagcgggaggagagcgcacttgactgtcggccaggcggccgcagggggaagtgggaatctgctcactgagagaagcactttcactgactcccagctggggaatgcagacatggagatgactctggagcgagcagtgagtatgctcgaggcagaccacatgctgccatccaggatttctgctgcagctactttcatacagcacgagtgcttccagaaatctgaagctcggaagagggttaaccagcttcgtggcatcctcaagcttctgcagctcctaaaagttcagaatgaagacgttcagcgagctgtgtgtggggccttgagaaacttagtatttgaagacaatgacaacaaattggaggtggctgaactaaatggggtacctcggctgctccaggtgctgaagcaaaccagagacttggagactaaaaaacaaataacaggtttgctgtggaatttgtcatctaatgacaaactcaagaatctcatgataacagaagcattgcttacgctgacggagaatatcatcatccccttttctgggtggcctgaaggagactacccaaaagcaaatggtttgctcgattttgacatattctacaacgtcactggatgcctaagaaacatgagttctgctggcgctgatgggagaaaagcgatgagaagatgtgacggactcattgactcactggtccattatgtcagaggaaccattgcagattaccagccagatgacaaggccacggagaattgtgtgtgcattcttcataacctctcctaccagctggaggcagagctcccagagaaatattcccagaatatctatattcaaaaccggaatatccagactgacaacaacaaaagtattggatgttttggcagtcgaagcaggaaagtaaaagagcaataccaggacgtgccgatgccggaggaaaagagcaaccccaagggcgtggagtggctgtggcattccattgttataaggatgtatctgtccttgatcgccaaaagtgtccgcaactacacacaagaagcatccttaggagctctgcagaacctcacggccggaagtggaccaatgccgacatcagtggctcagacagttgtccagaaggaaagtggcctgcagcacacccgaaagatgctgcatgttggtgacccaagtgtgaaaaagacagccatctcgctgctgaggaatctgtcccggaatctttctctgcagaatgaaattgccaaagaaactctccctgatttggtttccatcattcctgacacagtcccgagtactgaccttctcattgaaactacagcctctgcctgttacacattgaacaacataatccaaaacagttaccagaatgcacgcgaccttctaaacaccgggggcatccagaaaattatggccattagtgcaggcgatgcctatgcctccaacaaagcaagtaaagctgcttccgtccttctgtattctctgtgggcacacacggaactgcatcatgcctacaagaaggctcagtttaagaagacagattttgtcaacagccggactgccaaagcctaccactcccttaaagactgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgc

SEQ ID NO: 94 - DNA, 3861 bp, synthetic construct
  misc_feature 1..3861: note = Made in Lab - expression cassette polynucleotide
  source 1..3861: mol_type = other DNA
ctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcaggccaccatggcagcccccggcgccccagctgagtacggctacatccggaccgtcctgggccagcagatcctgggacaactggacagctccagcctggcgctgccctccgaggccaagctgaagctggcggggagcagcggccgcggcggccagacagtcaagagcctgcggatccaggagcaggtgcagcagaccctcgcccggaagggccgcagctccgtgggcaacggaaatcttcaccgaaccagcagtgttcctgagtatgtctacaacctacacttggttgaaaatgattttgttggaggccgttcccctgttcctaaaacctatgacatgctaaaggctggcacaactgccacttatgaaggtcgctggggaagaggaacagcacagtacagctcccagaagtccgtggaagaaaggtccttgaggcatcctctgaggagactggagatttctcctgacagcagcccggagagggctcactacacgcacagcgattaccagtacagccagagaagccaggctgggcacaccctgcaccaccaagaaagcaggcgggccgccctcctagtgccaccgagatatgctcgttccgagatcgtgggggtcagccgtgctggcaccacaagcaggcagcgccactttgacacataccacagacagtaccagcatggctctgttagcgacaccgtttttgacagcatccctgccaacccggccctgctcacgtaccccaggccagggaccagccgcagcatgggcaacctcttggagaaggagaactacctgacggcagggctcactgtcgggcaggtcaggccgctggtgcccctgcagcccgtcactcagaacagggcttccaggtcctcctggcatcagagctccttccacagcacccgcacgctgagggaagctgggcccagtgtcgccgtggattccagcgggaggagagcgcacttgactgtcggccaggcggccgcagggggaagtgggaatctgctcactgagagaagcactttcactgactcccagctggggaatgcagacatggagatgactctggagcgagcagtgagtatgctcgaggcagaccacatgctgccatccaggatttctgctgcagctactttcatacagcacgagtgcttccagaaatctgaagctcggaagagggttaaccagcttcgtggcatcctcaagcttctgcagctcctaaaagttcagaatgaagacgttcagcgagctgtgtgtggggccttgagaaacttagtatttgaagacaatgacaacaaattggaggtggctgaactaaatggggtacctcggctgctccaggtgctgaagcaaaccagagacttggagactaaaaaacaaataacaggtttgctgtggaatttgtcatctaatgacaaactcaagaatctcatgataacagaagcattgcttacgctgacggagaatatcatcatccccttttctgggtggcctgaaggagactacccaaaagcaaatggtttgctcgattttgacatattctacaacgtcactggatgcctaagaaacatgagttctgctggcgctgatgggagaaaagcgatgagaagatgtgacggactcattgactcactggtccattatgtcagaggaaccattgcagattaccagccagatgacaaggccacggagaattgtgtgtgcattcttcataacctctcctaccagctggaggcagagctcccagagaaatattcccagaatatctatattcaaaaccggaatatccagactgacaacaacaaaagtattggatgttttggcagtcgaagcaggaaagtaaaagagcaataccaggacgtgccgatgccggaggaaaagagcaaccccaagggcgtggagtggctgtggcattccattgttataaggatgtatctgtccttgatcgccaaaagtgtccgcaactacacacaagaagcatccttaggagctctgcagaacctcacggccggaagtggaccaatgccgacatcagtggctcagacagttgtccagaaggaaagtggcctgcagcacacccgaaagatgctgcatgttggtgacccaagtgtgaaaaagacagccatctcgctgctgaggaatctgtcccggaatctttctctgcagaatgaaattgccaaagaaactctccctgatttggtttccatcattcctgacacagtcccgagtactgaccttctcattgaaactacagcctctgcctgttacacattgaacaacataatccaaaacagttaccagaatgcacgcgaccttctaaacaccgggggcatccagaaaattatggccattagtgcaggcgatgcctatgcctccaacaaagcaagtaaagctgcttccgtccttctgtattctctgtgggcacacacggaactgcatcatgcctacaagaaggctcagtttaagaagacagattttgtcaacagccggactgccaaagcctaccactcccttaaagactgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgc

SEQ ID NO: 95 - DNA, 4369 bp, synthetic construct
  misc_feature 1..4369: note = Made in Lab - expression cassette polynucleotide
  source 1..4369: mol_type = other DNA
acccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcgccaccatggcagcccccggcgccccagctgagtacggctacatccggaccgtcctgggccagcagatcctgggacaactggacagctccagcctggcgctgccctccgaggccaagctgaagctggcggggagcagcggccgcggcggccagacagtcaagagcctgcggatccaggagcaggtgcagcagaccctcgcccggaagggccgcagctccgtgggcaacggaaatcttcaccgaaccagcagtgttcctgagtatgtctacaacctacacttggttgaaaatgattttgttggaggccgttcccctgttcctaaaacctatgacatgctaaaggctggcacaactgccacttatgaaggtcgctggggaagaggaacagcacagtacagctcccagaagtccgtggaagaaaggtccttgaggcatcctctgaggagactggagatttctcctgacagcagcccggagagggctcactacacgcacagcgattaccagtacagccagagaagccaggctgggcacaccctgcaccaccaagaaagcaggcgggccgccctcctagtgccaccgagatatgctcgttccgagatcgtgggggtcagccgtgctggcaccacaagcaggcagcgccactttgacacataccacagacagtaccagcatggctctgttagcgacaccgtttttgacagcatccctgccaacccggccctgctcacgtaccccaggccagggaccagccgcagcatgggcaacctcttggagaaggagaactacctgacggcagggctcactgtcgggcaggtcaggccgctggtgcccctgcagcccgtcactcagaacagggcttccaggtcctcctggcatcagagctccttccacagcacccgcacgctgagggaagctgggcccagtgtcgccgtggattccagcgggaggagagcgcacttgactgtcggccaggcggccgcagggggaagtgggaatctgctcactgagagaagcactttcactgactcccagctggggaatgcagacatggagatgactctggagcgagcagtgagtatgctcgaggcagaccacatgctgccatccaggatttctgctgcagctactttcatacagcacgagtgcttccagaaatctgaagctcggaagagggttaaccagcttcgtggcatcctcaagcttctgcagctcctaaaagttcagaatgaagacgttcagcgagctgtgtgtggggccttgagaaacttagtatttgaagacaatgacaacaaattggaggtggctgaactaaatggggtacctcggctgctccaggtgctgaagcaaaccagagacttggagactaaaaaacaaataacagaccatacagtcaatttaagaagtaggaatggctggccgggcgcggtggctcacgcctgtaatcccagcactttgggaggccaaggcgggcggatcacgaggtcaggagttcgagaccagcctgaccaacatggtttgctgtggaatttgtcatctaatgacaaactcaagaatctcatgataacagaagcattgcttacgctgacggagaatatcatcatccccttttctgggtggcctgaaggagactacccaaaagcaaatggtttgctcgattttgacatattctacaacgtcactggatgcctaagaaacatgagttctgctggcgctgatgggagaaaagcgatgagaagatgtgacggactcattgactcactggtccattatgtcagaggaaccattgcagattaccagccagatgacaaggccacggagaattgtgtgtgcattcttcataacctctcctaccagctggaggcagagctcccagagaaatattcccagaatatctatattcaaaaccggaatatccagactgacaacaacaaaagtattggatgttttggcagtcgaagcaggaaagtaaaagagcaataccaggacgtgccgatgccggaggaaaagagcaaccccaagggcgtggagtggctgtggcattccattgttataaggatgtatctgtccttgatcgccaaaagtgtccgcaactacacacaagaagcatccttaggagctctgcagaacctcacggccggaagtggaccaatgccgacatcagtggctcagacagttgtccagaaggaaagtggcctgcagcacacccgaaagatgctgcatgttggtgacccaagtgtgaaaaagacagccatctcgctgctgaggaatctgtcccggaatctttctctgcagaatgaaattgccaaagaaactctccctgatttggtttccatcattcctgacacagtcccgagtactgaccttctcattgaaactacagcctctgcctgttacacattgaacaacataatccaaaacagttaccagaatgcacgcgaccttctaaacaccgggggcatccagaaaattatggccattagtgcaggcgatgcctatgcctccaacaaagcaagtaaagctgcttccgtccttctgtattctctgtgggcacacacggaactgcatcatgcctacaagaaggctcagtttaagaagacagattttgtcaacagccggactgccaaagcctaccactcccttaaagactgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgc

SEQ ID NO: 96 - DNA, 3993 bp, synthetic construct
  misc_feature 1..3993: note = Made in Lab - expression cassette polynucleotide
  source 1..3993: mol_type = other DNA
ctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcaggccaccatggcagcccccggcgccccagctgagtacggctacatccggaccgtcctgggccagcagatcctgggacaactggacagctccagcctggcgctgccctccgaggccaagctgaagctggcggggagcagcggccgcggcggccagacagtcaagagcctgcggatccaggagcaggtgcagcagaccctcgcccggaagggccgcagctccgtgggcaacggaaatcttcaccgaaccagcagtgttcctgagtatgtctacaacctacacttggttgaaaatgattttgttggaggccgttcccctgttcctaaaacctatgacatgctaaaggctggcacaactgccacttatgaaggtcgctggggaagaggaacagcacagtacagctcccagaagtccgtggaagaaaggtccttgaggcatcctctgaggagactggagatttctcctgacagcagcccggagagggctcactacacgcacagcgattaccagtacagccagagaagccaggctgggcacaccctgcaccaccaagaaagcaggcgggccgccctcctagtgccaccgagatatgctcgttccgagatcgtgggggtcagccgtgctggcaccacaagcaggcagcgccactttgacacataccacagacagtaccagcatggctctgttagcgacaccgtttttgacagcatccctgccaacccggccctgctcacgtaccccaggccagggaccagccgcagcatgggcaacctcttggagaaggagaactacctgacggcagggctcactgtcgggcaggtcaggccgctggtgcccctgcagcccgtcactcagaacagggcttccaggtcctcctggcatcagagctccttccacagcacccgcacgctgagggaagctgggcccagtgtcgccgtggattccagcgggaggagagcgcacttgactgtcggccaggcggccgcagggggaagtgggaatctgctcactgagagaagcactttcactgactcccagctggggaatgcagacatggagatgactctggagcgagcagtgagtatgctcgaggcagaccacatgctgccatccaggatttctgctgcagctactttcatacagcacgagtgcttccagaaatctgaagctcggaagagggttaaccagcttcgtggcatcctcaagcttctgcagctcctaaaagttcagaatgaagacgttcagcgagctgtgtgtggggccttgagaaacttagtatttgaagacaatgacaacaaattggaggtggctgaactaaatggggtacctcggctgctccaggtgctgaagcaaaccagagacttggagactaaaaaacaaataacagaccatacagtcaatttaagaagtaggaatggctggccgggcgcggtggctcacgcctgtaatcccagcactttgggaggccaaggcgggcggatcacgaggtcaggagttcgagaccagcctgaccaacatggtttgctgtggaatttgtcatctaatgacaaactcaagaatctcatgataacagaagcattgcttacgctgacggagaatatcatcatccccttttctgggtggcctgaaggagactacccaaaagcaaatggtttgctcgattttgacatattctacaacgtcactggatgcctaagaaacatgagttctgctggcgctgatgggagaaaagcgatgagaagatgtgacggactcattgactcactggtccattatgtcagaggaaccattgcagattaccagccagatgacaaggccacggagaattgtgtgtgcattcttcataacctctcctaccagctggaggcagagctcccagagaaatattcccagaatatctatattcaaaaccggaatatccagactgacaacaacaaaagtattggatgttttggcagtcgaagcaggaaagtaaaagagcaataccaggacgtgccgatgccggaggaaaagagcaaccccaagggcgtggagtggctgtggcattccattgttataaggatgtatctgtccttgatcgccaaaagtgtccgcaactacacacaagaagcatccttaggagctctgcagaacctcacggccggaagtggaccaatgccgacatcagtggctcagacagttgtccagaaggaaagtggcctgcagcacacccgaaagatgctgcatgttggtgacccaagtgtgaaaaagacagccatctcgctgctgaggaatctgtcccggaatctttctctgcagaatgaaattgccaaagaaactctccctgatttggtttccatcattcctgacacagtcccgagtactgaccttctcattgaaactacagcctctgcctgttacacattgaacaacataatccaaaacagttaccagaatgcacgcgaccttctaaacaccgggggcatccagaaaattatggccattagtgcaggcgatgcctatgcctccaacaaagcaagtaaagctgcttccgtccttctgtattctctgtgggcacacacggaactgcatcatgcctacaagaaggctcagtttaagaagacagattttgtcaacagccggactgccaaagcctaccactcccttaaagactgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgc

(19) United States
(12) Patent Application Publication
(10) Pub. No.: US 2024/0390519 A1
(43) Pub. Date: Nov. 28, 2024

(54) PLAKOPHILIN-2 (PKP2) GENE THERAPY USING AAV VECTOR

(71) Applicant: Spacecraft Seven, LLC, Cranbury, NJ (US)

(72) Inventors: Christopher Dean HERZOG, Fanwood, NJ (US); Chester Bittencort SACRAMENTO, Cranbury, NJ (US); Raj PRABHAKAR, Cranbury, NJ (US); David RICKS, Cranbury, NJ (US)

(21) Appl. No.: 18/399,438

(22) Filed: Dec. 28, 2023

Related U.S. Application Data
(63) Continuation of application No. 17/670,390, filed on Feb. 11, 2022, now abandoned.
Application No. 17/670,390 is a continuation of application No. PCT/US2021/045220, filed on Aug. 9, 2021.
(60) Provisional application No. 63/063,032, filed on Aug. 7, 2020.

(51) Int. Cl. A61K 48/00; A61K 38/00; C07K 14/005; C12N 15/113; C12N 15/86

(52) U.S. Cl. CPC A61K 48/005; C07K 14/005; C12N 15/113; C12N 15/86; A61K 38/00; C12N 2750/14143

(57) ABSTRACT

Provided herein is a gene therapy for PKP2 (Plakophilin-2), e.g. using an adeno-associated virus (AAV) vector. The promoter of the vector may be a MHCK7 promoter or a cardiac troponin T (HTNNT2) promoter. The capsid may be an AAV9 or AAVrh74 capsid or a functional variant thereof. Other promoters or capsids may be used. Further provided are methods of treatment, such as by intravenous, intracoronary, intracarotid or intracardiac administration of the rAAV vector, and other compositions and methods.

### CROSS-REFERENCE TO RELATED APPLICATIONS

This application is a continuation of U.S. application Ser. No. 17/670,390, filed Feb. 11, 2022, which is a continuation of International Application No. PCT/US2021/045220, filed Aug. 9, 2021, which claims the benefit of U.S. Provisional Application No. 63/063,032, filed Aug. 7, 2020, which are incorporated by reference herein in their entireties.

### STATEMENT REGARDING THE SEQUENCE LISTING

The contents of the electronic sequence listing (ROPA_021_05US_SeqList_ST26.xml; Size: 221,873 bytes; and Date of Creation: Dec. 26, 2023) are herein incorporated by reference in its entirety.

### BACKGROUND

Arrhythmogenic right ventricular cardiomyopathy (ARVC) is a form of adult-onset heart disease, which impacts an estimated 1 in 1,000 to 1 in 1,250 people. It manifests as breakdown of the muscular wall of the heart (the myocardium) over time, which leads to increased risk of abnormal heartbeat (arrhythmia) and an increased risk of sudden death when an affected individual exercises strenuously. Individuals may also experience a sensation of fluttering or pounding in the chest (palpitations), light-headedness, fainting (syncope), shortness of breath, and abnormal swelling in the legs or abdomen. Over time, ARVC can lead to heart failure.

At least 13 genes are implicated in ARVC, many of which are involved in the biogenesis of desmosomes, which are intracellular junctions that provide strong adhesion between cells. When desmosomes fail to form properly, myocardial cells may detach from one another and die. The right ventricle in particular may develop weakness, while fatty deposits and scar tissue may replace the damaged myocardium, leading to distension of the right ventricle. These alterations ultimately prevent effective heart pumping and disrupt the electrical signals that control the heartbeat, leading to arrhythmia. Autosomal dominant plakophilin-2 (PKP2) cardiomyopathy is an inherited ARVC in which mutations affecting PKP2 are detected.

There remains, therefore, an unmet need in the art for treatments for PKP2-related diseases and disorders, including ARVC. The compositions and methods disclosed herein address this need.

### SUMMARY

The present invention relates generally to gene therapy for a disease or disorder, e.g., a cardiac disease or disorder, using a vector expressing PKP2 or a functional variant thereof.

In one aspect, the disclosure provides polynucleotide, comprising an expression cassette and optionally flanking adeno-associated virus (AAV) inverted terminal repeats (ITRs), wherein the polynucleotide comprises a polynucleotide sequence encoding a Plakophilin-2 (PKP2) or a functional variant thereof, operatively linked to a promoter.

In some embodiments, the promoter is a cardiac-specific promoter.

In some embodiments, the promoter is a muscle-specific promoter.

In some embodiments, the promoter is a cardiomyocyte-specific promoter.

In some embodiments, the promoter is a Myosin Heavy-chain Creatine Kinase 7 (MHCK7) promoter.

In some embodiments, the MHCK7 promoter shares at least 75%, 80%, 85%, 90%, 95%, 96%, 97%, 98%, 99% or 100% identity with SEQ ID NO: 31.

In some embodiments, the promoter is a cardiac troponin T (hTNNT2) promoter.

In some embodiments, the hTNNT2 promoter shares at least 75%, 80%, 85%, 90%, 95%, 96%, 97%, 98%, 99% or 100% identity with SEQ ID NO: 32.

In some embodiments, the expression cassette comprises exon 1 of the cardiac troponin T (hTNNT2) gene, wherein optionally the hTNNT2 promoter and exon 1 together share at least 75%, 80%, 85%, 90%, 95%, 96%, 97%, 98%, 99% or 100% identity with SEQ ID NO: 32.

In some embodiments, the promoter is a ubiquitous promoter, optionally a CMV promoter or a CAG promoter.

In some embodiments, the expression cassette comprises a polyA signal.

In some embodiments, the polyA signal is a human growth hormone (hGH) polyA.

In some embodiments, the expression cassette comprises a Woodchuck Hepatitis Virus Posttranscriptional Regulatory Element (WPRE), optionally a WPRE(x).

In some embodiments, the Plakophilin-2 (PKP2) or functional variant thereof is a PKP2.

In some embodiments, the PKP2 is a functional PKP2.

In some embodiments, the PKP2 is a human PKP2.

In some embodiments, the PKP2 is PKP2 isoform A.

In some embodiments, the PKP2 isoform A shares at least 75%, 80%, 85%, 90%, 95%, 96%, 97%, 98%, 99% or 100% identity with SEQ ID NO: 1.

In some embodiments, the PKP2 is PKP2 isoform B.

In some embodiments, the PKP2 shares at least 75%, 80%, 85%, 90%, 95%, 96%, 97%, 98%, 99% or 100% identity with SEQ ID NO: 2.

In some embodiments, the polynucleotide sequence encoding PKP2 is a human PKP2 polynucleotide.

In some embodiments, the polynucleotide sequence encoding PKP2 shares at least 75%, 80%, 85%, 90%, 95%, 96%, 97%, 98%, 99% or 100% identity with SEQ ID NO: 3.

In some embodiments, the polynucleotide sequence encoding PKP2 shares at least 75%, 80%, 85%, 90%, 95%, 96%, 97%, 98%, 99% or 100% identity with SEQ ID NO: 4.

In some embodiments, the polynucleotide comprises at least about 4.0 kb, at least about 4.1 kb, at least about 4.2 kb, at least about 4.3 kb, at least about 4.4 kb, or at least about 4.5 kb.

In some embodiments, the polynucleotide comprises at most about 4.1 kb, at most about 4.2 kb, at most about 4.3 kb, at most about 4.4 kb, at most about 4.5 kb, or at most about 4.6 kb.

In some embodiments, the polynucleotide comprises 4.0 kb to 4.6 kb, 4.0 kb to 4.5 kb, or 4.0 kb to 4.4 kb or wherein the polynucleotide comprises 4.0 kb to 4.3 kb, 4.0 kb to 4.2 kb, or 4.0 kb to 4.1 kb.

In some embodiments, the PKP2 or functional variant thereof comprises at least 800 or at least 830 amino acids.

In some embodiments, the polynucleotide shares at least 75%, 80%, 85%, 90%, 95%, 96%, 97%, 98%, 99% or 100% identity with any one of SEQ ID NOs: 8-15.

In some embodiments, the expression cassette is flanked by 5′ and 3′ inverted terminal repeats (ITRs)

In some embodiments, the ITRs are AAV2 ITRs and/or the ITRs share at least 75%, 80%, 85%, 90%, 95%, 96%, 97%, 98%, 99% or 100% identity with any one of SEQ ID NO: 20-26.

In another aspect, the disclosure provides a gene therapy vector, comprising the polynucleotide of any one of the preceding embodiments.

In some embodiments, the gene therapy vector is a recombinant adeno-associated virus (rAAV) vector.

In some embodiments, the rAAV vector is an AAV9 or a functional variant thereof.

In some embodiments, the rAAV vector comprises a capsid protein that shares 90%, 91%, 92%, 93%, 94%, 95%, 96%, 97%, 98%, 99% or 100% identity to any one of SEQ ID NO: 77.

In some embodiments, the rAAV vector is an AAVrh10 or a functional variant thereof.

In some embodiments, the rAAV vector comprises a capsid protein that shares 90%, 91%, 92%, 93%, 94%, 95%, 96%, 97%, 98%, 99% or 100% identity to any one of SEQ ID NO: 79.

In some embodiments, the rAAV vector is an AAV6 or a functional variant thereof.

In some embodiments, the rAAV vector comprises a capsid protein that shares 90%, 91%, 92%, 93%, 94%, 95%, 96%, 97%, 98%, 99% or 100% identity to any one of SEQ ID NO: 78.

In some embodiments, the rAAV vector is an AAVrh74 or a functional variant thereof.

In some embodiments, the rAAV vector comprises a capsid protein that shares 90%, 91%, 92%, 93%, 94%, 95%, 96%, 97%, 98%, 99% or 100% identity to any one of SEQ ID NO: 80.

In another aspect, the disclosure provides a method of treating and/or preventing a disease or disorder in a subject in need thereof, comprising administering the vector of any one of the preceding embodiments to the subject.

In some embodiments, the disease or disorder is a cardiac disorder.

In some embodiments, the disease or disorder is cardiomyopathy.

In some embodiments, the cardiomyopathy is arrhythmogenic right ventricular cardiomyopathy (ARVC).

In some embodiments, the cardiomyopathy is hypertrophic cardiomyopathy or dilated cardiomyopathy.

In some embodiments, the disease or disorder is characterized by fibrofatty infiltration of myocardium.

In some embodiments, the disease or disorder is heart failure.

In some embodiments, the subject is a mammal.

In some embodiments, the subject is a primate.

In some embodiments, the subject is a human.

In some embodiments, the subject has a mutation in a PRP2 gene.

In some embodiments, the vector is administered by intravenous injection, intracardiac injection, intracardiac infusion, and/or cardiac catheterization.

In some embodiments, the administration increases PKP2 expression by at least about 5%.

In some embodiments, the administration increases PKP2 expression by at least about 30%.

In some embodiments, the administration increases PKP2 expression by at least about 70%.

In some embodiments, the administration increases PKP2 expression by about 5% to about 10%.

In some embodiments, the administration increases PKP2 expression by about 30% to about 50%.

In some embodiments, the administration increases PKP2 expression by about 50% to about 70%.

In some embodiments, the administration increases PKP2 expression by about 70% to about 100%.

In some embodiments, the method treats and/or prevents the disease or disorder.

In some embodiments, the method comprises administering an effective amount of the vector.

In some embodiments, the disease or disorder is related to or caused by loss of function in PKP2 in the subject.

In some embodiments, the disease or disorder is related to or caused by gain of function in PKP2 in the subject.

In some embodiments, the subject has a mutation that causes an amino acid substitution selected from p.Arg490Trp, Asp26Asn, Thr50_Val51SerfsX60, Arg79X, Tyr86X, Gln133X, Val406SerfsX3, Tyr616X, Trp676X, Cys796Arg, Cys796E, Tyr807X, Glu62Lys, S688P, Trp848X, Y86X, V406X, Y616X, W848X, and Y807X, relative to a human PKP2 gene encoding a human PKP2 having the sequence of SEQ ID NO: 2.

In some embodiments, the method comprises administering a pharmaceutical composition comprising an effective amount of the vector.

In some embodiments, the method comprises administering between about 1×10¹¹ vector genomes and about 1×10¹³ vector genomes of the vector to the subject, administering between about 1×10¹² vector genomes and about 1×10¹⁴ vector genomes of the vector to the subject, or administering between about 1×10¹³ vector genomes and about 1×10¹⁵ vector genomes of the vector to the subject.

In another aspect, the disclosure provides a pharmaceutical composition comprising the vector of any one of the preceding embodiments.

In another aspect, the disclosure provides a kit comprising the vector of any one of the preceding embodiments or the pharmaceutical composition of the preceding embodiment and optionally instructions for use.

In another aspect, the disclosure provides a use of the vector of any one of the preceding embodiments in treating a disease or disorder, optionally according to the method of any one of the preceding embodiments.

In another aspect, the disclosure provides a vector according to any one of the preceding embodiments for use in treating a disease or disorder, optionally according to the method of any one of the preceding embodiments.

In another aspect, the disclosure provides a polynucleotide, comprising a polynucleotide sequences that shares at least 90%, 91%, 92%, 93%, 94%, 95%, 96%, 97%, 98%, 99%, or 100% identity to any one of SEQ ID NOs: 12-15 and 89-92 or to any one of SEQ ID NOs: 8-11 and 93-96.

In some embodiments, the promoter is a MHCK7 promoter.

In some embodiments, the MHCK7 promoter shares at least 75%, 80%, 85%, 90%, 95%, 96%, 97%, 98%, 99% or 100% identity with SEQ ID NO: 31.

In some embodiments, the PKP2 is a human PKP2.

In some embodiments, the PKP2 is PKP2 isoform A.

In some embodiments, the PKP2 isoform A shares at least 80%, 90%, 95%, 99% or 100% identity with SEQ ID NO: 1.

In another aspect, the disclosure provides a gene therapy vector, comprising the polynucleotide of any one of the preceding embodiments.

In some embodiments, the gene therapy vector is a recombinant adeno-associated virus (rAAV) vector.

In some embodiments, the rAAV vector is an AAV9 vector.

In some embodiments, the rAAV vector is an AAVrh74 vector.

In another aspect, the disclosure provides a method of treating and/or preventing a cardiac disorder in a subject identified as having a mutation in the PRP2 gene, comprising administering the vector of any one of the preceding embodiments to the subject.

In some embodiments, the cardiac disorder is cardiomyopathy, optionally arrhythmogenic right ventricular cardiomyopathy (ARVC), hypertrophic cardiomyopathy, or dilated cardiomyopathy.

In some embodiments, the cardiac disorder is heart failure.

In some embodiments, the subject is a mammal.

In some embodiments, the vector is administered by intravenous injection, intracardiac injection, intracardiac infusion, and/or cardiac catheterization.

In some embodiments, the method prevents or reduces a decrease in left ventricle ejection fraction percentage (LVEF %), optionally by about 50%, about 60%, about 70%, about 80%, about 90%, or about 100% compared to the decrease observed in an untreated subject identified as having a mutation in the PRP2 gene.

In some embodiments, the method prevents or reduces a decrease in left ventricle fractional shortening percentage (FS %), optionally by about 50%, about 60%, about 70%, about 80%, about 90%, or about 100% compared to the decrease observed in an untreated subject identified as having a mutation in the PRP2 gene.

In some embodiments, the method prevents or reduces an increase in right ventricle area in millimeters squared (RV Area (mm2), optionally by about 50%, about 60%, about 70%, about 80%, about 90%, or about 100% compared to the increase observed in an untreated subject identified as having a mutation in the PRP2 gene.

In some embodiments, the method prevents or reduces a decrease in right ventricle velocity time integral in millimeters per second (RV VTI (mm/sec), optionally by about 50%, about 60%, about 70%, about 80%, about 90%, or about 100% compared to the decrease observed in an untreated subject identified as having a mutation in the PRP2 gene.

In some embodiments, the method prevents or reduces an increase in left ventricle or right ventricle fibrosis, optionally by about 50%, about 60%, about 70%, about 80%, about 90%, or about 100% compared to the increase observed in an untreated subject identified as having a mutation in the PRP2 gene.

Various other aspects and embodiments are disclosed in the detailed description that follows. The invention is limited solely by the appended claims.

### BRIEF DESCRIPTION OF FIGURES

FIG. 1 shows a diagram illustrating a non-limiting example of a vector genome. The full polynucleotide sequence of the vector genome is SEQ ID NO: 12. The expression cassette is SEQ ID NO: 8. The MHCK7 promoter as described herein is labelled “Enhancer/MHCK7” in the diagram.

FIG. 2 shows a diagram illustrating a non-limiting example of a vector genome. The full polynucleotide sequence of the vector genome is SEQ ID NO: 13. The expression cassette is SEQ ID NO: 9.

FIG. 3 shows a diagram illustrating a non-limiting example of a vector genome. The full polynucleotide sequence of the vector genome is SEQ ID NO: 14. The expression cassette is SEQ ID NO: 10. The MHCK7 promoter as described herein is labelled “Enhancer/MHCK7” in the diagram.

FIG. 4 shows a diagram illustrating a non-limiting example of a vector genome. The full polynucleotide sequence of the vector genome is SEQ ID NO: 15. The expression cassette is SEQ ID NO: 11.

FIGS. 5A-5B show PKP2 protein expression in transduced differentiated AC16 cells. FIG. 5A shows Western Blots (WB) of PKP2 (top panel) or loading control, GAPDH (bottom panel). FIG. 5B show a bar graph of the Western Blot. The AAV vector serotype (AAV9 or rh74) and the promoter (MHCK7 or hTnT) are noted. Controls included a GFP vector (CON-GFP) and no transduction (No Tdxn).

FIGS. 6A-6D show left ventricle ejection fraction percentage (LVEF %) for normal mice (left bars), untreated PKP2 knockout mice (middle bars, cKO PKP2), or treated mice (right bars). FIG. 6A shows results with the AAV9 vector and MHCK7 promoter (AAV9-MHCK7). FIG. 6B shows results with the AAVrh.74 vector and MHCK7 promoter (AAVrh.74-MHCK7). FIG. 6C shows results with the AAV9 vector and hTnT promoter (AAV9-hTnT). FIG. 6D shows results with the AAVrh.74 vector and hTnT promoter (AA Vrh.74-hTnT).

FIGS. 7A-7D show left ventricle fractional shortening percentage (FS %) for normal mice (left bars), untreated PKP2 knockout mice (middle bars, cKO PKP2), or treated mice (right bars). FIG. 7A shows results with the AAV9 vector and MHCK7 promoter (AAV9-MHCK7). FIG. 7B shows results with the AAVrh.74 vector and MHCK7 promoter (AA Vrh.74-MHCK7). FIG. 7C shows results with the AAV9 vector and hTnT promoter (AAV9-hTnT). FIG. 7D shows results with the AAVrh.74 vector and the hTnT promoter (AAVrh.74-hTnT).

FIGS. 8A-8D show right ventricle area in millimeters squared (RV Area (mm²)) for normal mice (left bars), untreated PKP2 knockout mice (middle bars, cKO PKP2), or treated mice (right bars). FIG. 8A shows results with the AAV9 vector and MHCK7 promoter (AAV9-MHCK7). FIG. 8B shows results with the AAVrh.74 vector and MHCK7 promoter (AA Vrh.74-MHCK7). FIG. 8C shows results with the AAV9 vector and hTnT promoter (AAV9-hTnT). FIG. 8D shows results with the AAVrh.74 vector and hTnT promoter (AA Vrh.74-hTnT).

FIGS. 9A-9D show right ventricle velocity time integral in millimeters per second (RV VTI (mm/sec)) for normal mice (left bars), untreated PKP2 knockout mice (middle bars, cKO PKP2), or treated mice (right bars). FIG. 9A shows results with the AAV9 vector and MHCK7 promoter (AAV9-MHCK7). FIG. 9B shows results with the AAVrh.74 vector and MHCK7 promoter (AA Vrh.74-MHCK7). FIG. 9C shows results with the AAV9 vector and hTnT promoter (AAV9-hTnT). FIG. 9D shows results with the AAVrh.74 vector and hTnT promoter (AAVrh.74-hTnT).

FIGS. 10A-10B illustrate the degree of fibrosis in left and right ventricles based on quantitation of the Percent Collagen following trichrome histological staining of the heart. Control animals without the conditional PKP2 gene knock-out (Cre Neg group) were found to have very little collagen, while control PKP2 knock-out animals receiving Formulation Buffer (CKO FB group) were found to have substantially greater proportion of collagen in both left and right ventricles. AAV-mediated overexpression of PKP2 resulted in robust attenuation of collagen, to varying degrees, in all AAV-injected groups [n=4 for all groups; p-values reflect results from One-way ANOVA with Bonferroni post-hoc analyses]. FIG. 10A is a bar graph of percent collagen in the left ventricle. FIG. 10B is a bar graph of percent collagen in the right ventricle.

FIG. 11 shows a diagram illustrating a non-limiting example of a vector genome. The full polynucleotide sequence of the vector genome is SEQ ID NO: 89. The expression cassette is SEQ ID NO: 93. The MHCK7 promoter as described herein is labelled “Enhancer/MHCK7” in the diagram.

FIG. 12 shows a diagram illustrating a non-limiting example of a vector genome. The full polynucleotide sequence of the vector genome is SEQ ID NO: 90. The expression cassette is SEQ ID NO: 94.

FIG. 13 shows a diagram illustrating a non-limiting example of a vector genome. The full polynucleotide sequence of the vector genome is SEQ ID NO: 91. The expression cassette is SEQ ID NO: 95. The MHCK7 promoter as described herein is labelled “Enhancer/MHCK7” in the diagram.

FIG. 14 shows a diagram illustrating a non-limiting example of a vector genome. The full polynucleotide sequence of the vector genome is SEQ ID NO: 92. The expression cassette is SEQ ID NO: 96.

### DETAILED DESCRIPTION OF THE INVENTION

The present disclosure provided gene therapy vectors for PKP2 that deliver a polynucleotide encoding a PKP2 polypeptide or a functional variant thereof, along with methods of use, and other compositions and methods. In particular embodiments, the disclosure relates to a gene therapy vector comprising a promoter sequence operatively linked to a polynucleotide encoding a PKP2 polypeptide or a functional variant thereof. In some embodiments, the promoter is a Myosin Heavy-chain Creatine Kinase 7 (MHCK7) promoter. In some embodiment, the AAV vector is an AAV9 vector. In some embodiments, the promoter is an MHCK7 promoter and the AAV vector is an AAV9 vector. In some embodiments, the promoter is a hTNNT2 promoter. In some embodiment, the promoter is an hTNNT2 promoter and the AAV vector is an AAV9 vector. In some embodiments, the PKP2 is human PKP2a. In some embodiments, the PKP2 is human PKP2b. In some embodiment, the AAV vector is an rh74 vector. In some embodiments, the promoter is an MHCK7 promoter and the AAV vector is an rh74 vector. In some embodiments, the promoter is a hTNNT2 promoter. In some embodiment, the promoter is an hTNNT2 promoter and the AAV vector is an rh74 vector. In some embodiments, the PKP2 is human PKP2a. In some embodiments, the PKP2 is human PKP2b.

This disclosure further provides methods of treating a disorder or disorder in a subject by administering a gene therapy vector of the disclosure to the subject. In a certain embodiment, the disorder or disorder is arrhythmogenic right ventricular cardiomyopathy (ARVC).

In certain embodiments, the subject being treated is an ARVC patient having one or more mutation in a PKP2 gene. More than half of ARVC patients carry mutations in the desmosomal gene PKP2 encoding the protein Plakophilin-2 (PKP2). PKP2 is also associated with Brugada syndrome (BrS) and idiopathic ventricular fibrillation. It is a member of the armadillo repeat and plakophilin protein family. The protein contains nine central, conserved armadillo repeat domains flanked by N-terminal and C-terminal domains. It functions to link cadherins to intermediate filaments in the cytoskeleton

Plakophilin 2 localizes to cell desmosomes and nuclei and binds plakoglobin, desmoplakin, and the desmosomal cadherins via an N-terminal head domain. PKP2 provides a lateral stabilizing force with the desmosomal-intermediate filament assembly facilitating cell-to-cell contact. It may also serve roles in intracellular signaling regulation, electrophysiologic and trafficking regulation, and control of transcription processes.

Intravenous injection of an AAV9 vector encoding a C-terminal deletion mutant of PKP2a (R735X) in the heart of wild-type mice accelerates the appearance of ARVC when treated mice are subjected to exercise training. Cruz et al. J Am Coll Cardiol. 65(14):1438-50 (2015). Mutant PKP2a causes a disease phenotype; and control AAV9 vector expressing the non-mutant PKP2a causes no phenotypic change in wild type mice. Heterologous expression of wild-type human PKP2a does not induce disease or altered function. These studies demonstrate that mutant PKP2a can cause a disease phenotype. They fail to demonstrate a curative role for PKP2, because heterologous expression of non-mutant PKP2 resulted in no phenotypic change in the wild-type mouse.

In accordance with the present invention, a polynucleotide encoding a PKP2 or functional variant thereof, wherein the PKP2 or functional variant thereof comprises at least 800 or at least 830 amino acids (e.g., no C terminal truncation at Arg-735), may be employed in generating a gene therapy vector. The resulting vector may be employed in treating diseases or disorders, e.g. a PKP2-related disease or disorder, e.g. ARVC, Brugada syndrome (BrS), idiopathic ventricular fibrillation, dilated cardiomyopathy (DCM), and others.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

All publications and patents mentioned herein are hereby incorporated by reference in their entirety as if each individual publication or patent was specifically and individually indicated to be incorporated by reference. In case of conflict, the present application, including any definitions herein, will control. However, mention of any reference, article, publication, patent, patent publication, and patent application cited herein is not, and should not be taken as an acknowledgment, or any form of suggestion, that they constitute valid prior art or form part of the common general knowledge in any country in the world.

In the present description, any concentration range, percentage range, ratio range, or integer range is to be understood to include the value of any integer within the recited range and, when appropriate, fractions thereof (such as one tenth and one hundredth of an integer), unless otherwise indicated. The term “about”, when immediately preceding a number or numeral, means that the number or numeral ranges plus or minus 10%. It should be understood that the terms “a” and “an” as used herein refer to “one or more” of the enumerated components unless otherwise indicated. The use of the alternative (e.g., “or”) should be understood to mean either one, both, or any combination thereof of the alternatives. The term “and/or” should be understood to mean either one, or both of the alternatives. As used herein, the terms “include” and “comprise” are used synonymously.

As used herein, the terms “identity” and “identical” refer, with respect to a polypeptide or polynucleotide sequence, to the percentage of exact matching residues in an alignment of that “query” sequence to a “subject” sequence, such as an alignment generated by the BLAST algorithm. Identity is calculated, unless specified otherwise, across the full length of the subject sequence. Thus a query sequence “shares at least x % identity to” a subject sequence if, when the query sequence is aligned to the subject sequence, at least x % (rounded down) of the residues in the subject sequence are aligned as an exact match to a corresponding residue in the query sequence. Where the subject sequence has variable positions (e.g., residues denoted X), an alignment to any residue in the query sequence is counted as a match. Sequence alignments may be performed using the NCBI Blast service (BLAST+ version 2.12.0).

As used herein, the term “operatively linked” refers to a functional relationship between two or more nucleic acid (e.g., DNA) segments. Typically, it refers to the functional relationship of a transcriptional regulatory sequence to a transcribed sequence. For example, a promoter sequence is operatively linked to a coding sequence if it stimulates or modulates the transcription of the coding sequence in an appropriate host cell or other expression system. Generally, promoter transcriptional regulatory sequences that are operatively linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

As used herein, an “AAV vector” or “rAAV vector” refers to a recombinant vector comprising one or more polynucleotides of interest (or transgenes) that are flanked by AAV inverted terminal repeat sequences (ITRs). Such AAV vectors can be replicated and packaged into infectious viral particles when present in a host cell that has been transfected with a plasmid encoding and expressing rep and cap gene products. Alternatively, AAV vectors can be packaged into infectious particles using a host cell that has been stably engineered to express rep and cap genes.

As used herein, an “AAV virion” or “AAV viral particle” or “AAV vector particle” refers to a viral particle composed of at least one AAV capsid protein and an encapsidated polynucleotide AAV vector. As used herein, if the particle comprises a heterologous polynucleotide (i.e., a polynucleotide other than a wild-type AAV genome such as a transgene to be delivered to a mammalian cell), it is typically referred to as an “AAV vector particle” or simply an “AAV vector.” Thus, production of AAV vector particle necessarily includes production of AAV vector, as such a vector is contained within an AAV vector particle.

As used herein, “promoter” refers to a polynucleotide sequence capable of promoting initiation of RNA transcription from a polynucleotide in a eukaryotic cell.

As used herein, “vector genome” refers to the polynucleotide sequence packaged by the vector (e.g., an rAAV virion), including flanking sequences (in AAV, inverted terminal repeats). The terms “expression cassette” and “polynucleotide cassette” refer to the portion of the vector genome between the flanking ITR sequences. “Expression cassette” implies that the vector genome comprises at least one gene encoding a gene product operatively linked to an element that drives expression (e.g., a promoter).

As used herein, the term “patient in need” or “subject in need” refers to a patient or subject at risk of, or suffering from, a disease, disorder or condition that is amenable to treatment or amelioration with a recombinant gene therapy vector or gene editing system disclosed herein. A patient or subject in need may, for instance, be a patient or subject diagnosed with a disorder associated with heart. A subject may have a mutation in an PKP2 gene or deletion of all or a part of PKP2 gene, or of gene regulatory sequences, that causes aberrant expression of the PKP2 protein. “Subject” and “patient” are used interchangeably herein. The subject treated by the methods described herein may be an adult or a child. Subjects may range in age.

As used herein, the term “variant” refers to a protein that has one or more amino-acid substitution, insertion, or deletion as compared to a parental protein. As used herein, the term “functional variant” refers to a protein that has one or more amino-acid substitution, insertion, or deletion as compared to a parental protein, and which retains one or more desired activities of the parental protein.

As used herein, “treating” refers to ameliorating one or more symptoms of a disease or disorder. The term “preventing” refers to delaying or interrupting the onset of one or more symptoms of a disease or disorder or slowing the progression of PKP2-related disease or disorder, e.g., arrhythmogenic right ventricular cardiomyopathy (ARVC).

Adeno-associated virus (AAV) is a replication-deficient parvovirus, the single-stranded DNA genome of which is about 4.7 kb in length including two ˜145-nucleotide inverted terminal repeat (ITRs). There are multiple known variants of AAV, also sometimes called serotypes when classified by antigenic epitopes. The nucleotide sequences of the genomes of the AAV serotypes are known. For example, the complete genome of AAV-1 is provided in GenBank Accession No. NC_002077; the complete genome of AAV-2 is provided in GenBank Accession No. NC_001401 and Srivastava et al., J. Virol., 45:555-564 (1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al., J. Virol., 78:6381-6388 (2004); the AAV-10 genome is provided in Mol. Ther., 13(1): 67-76 (2006); and the AAV-11 genome is provided in Virology, 330(2): 375-383 (2004). The sequence of the AAVrh.74 genome is provided in U.S. Pat. No. 9,434,928, incorporated herein by reference. Cis-acting sequences directing viral DNA replication (rep), encapsidation/packaging and host cell chromosome integration are contained within the AAV ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (rep78, rep68, rep52, and rep40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158:97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is inserted as cloned DNA in plasmids, which makes construction of recombinant genomes feasible. Furthermore, because the signals directing AAV replication and genome encapsidation are contained within the ITRs of the AAV genome, some or all of the internal approximately 4.3 kb of the genome (encoding replication and structural capsid proteins, rep-cap) may be replaced with foreign DNA. To generate AAV vectors, the rep and cap proteins may be provided in trans. Another significant feature of AAV is that it is an extremely stable and hearty virus. It easily withstands the conditions used to inactivate adenovirus (56° to 65° C. for several hours), making cold preservation of AAV less critical. AAV may even be lyophilized. Finally, AAV-infected cells are not resistant to superinfection.

Gene delivery viral vectors useful in the practice of the present invention can be constructed utilizing methodologies well known in the art of molecular biology. Typically, viral vectors carrying transgenes are assembled from polynucleotides encoding the transgene, suitable regulatory elements and elements necessary for production of viral proteins, which mediate cell transduction. Such recombinant viruses may be produced by techniques known in the art, e.g., by transfecting packaging cells or by transient transfection with helper plasmids or viruses. Typical examples of virus packaging cells include but are not limited to HeLa cells, SF9 cells (optionally with a baculovirus helper vector), 293 cells, etc. A Herpesvirus-based system can be used to produce AAV vectors, as described in US20170218395A1. Detailed protocols for producing such replication-defective recombinant viruses may be found for instance in W095/14785, W096/22378, U.S. Pat. Nos. 5,882,877, 6,013,516, 4,861,719, 5,278,056 and W094/19478, the complete contents of each of which is hereby incorporated by reference.

The present disclosure contemplates compositions and methods of use related to Plakophilin-2 (PKP2) proteins or polypeptides. Various mutations in PKP2 are known to be associated with cardiomyopathy and heart failure, including diseases like those described in van Tintelen et al. Circulation 113:1650-58 (2006); Novelli Front. Cardiovasc. Med. (2008); and in other sources. Viral vector-mediated delivery of the PKP2 gene may therefore serve as a viable therapeutic for PKP2-related human diseases such as cardiomyopathy and heart failure.

More than 230 mutations in the PKP2 gene have been identified in people with arrhythmogenic right ventricular cardiomyopathy (ARVC). (See “PKP2 gene,” MedlinePlus). This condition most commonly affects the myocardium surrounding the right ventricle, one of the two lower chambers of the heart. ARVC increases the risk of an abnormal heartbeat (arrhythmia) and sudden death. Some PKP2 gene mutations lead to the production of an abnormally short version of plakophilin 2. Other mutations alter the structure of plakophilin 2 by adding, deleting, or changing one or more of its protein building blocks (amino acids). Studies suggest that the altered protein impairs the formation and function of desmosomes.

Without normal desmosomes, cells of the myocardium detach from one another and die, particularly when the heart muscle is placed under stress (such as during vigorous exercise). The damaged myocardium is gradually replaced by fat and scar tissue. As this abnormal tissue builds up, the walls of the right ventricle become stretched out, preventing the heart from pumping blood effectively. These changes also disrupt the electrical signals that control the heartbeat, which can lead to arrhythmia. Description of PKP2-related disease may be found in the following references: Bonne et al. Genomics 51:452-454 (1998) [PubMed: 9721216]; Bonne et al. Cytogenet. Cell Genet. 88:286-287 (2000) [PubMed: 10828611]; Dalal et al., Circulation 113:1641-1649 (2006) [PubMed: 16549640]; Gerull et al. Nature Genet. 36:1162-1164 (2004 [PubMed: 15489853]; Grossmann et al. J. Cell Biol. 167:149-160 (2004) [PubMed: 15479741]; Marcus et al. Circulation 65:384-398 (1982) [PubMed: 7053899]; and Mertens et al. J. Cell Biol. 135:1009-1025 (1996) [PubMed: 8922383]. See also OMIM.org entry 602861 (“PLAKOPHILIN 2; PKP2”).

The native sequences of human PKP2a and its isoform PKP2b are shown below, with Arg-735 underlined:

PKP2a-837 amino acids
(SEQ ID NO: 1)

| | |
|---|---|
| 1 | MAAPGAPAEY GYIRTVLGQQ ILGQLDSSSL ALPSEAKLKL |
| 41 | AGSSGRGGQT VKSLRIQEQV QQTLARKGRS SVGNGNLHRT |
| 81 | SSVPEYVYNL HLVENDFVGG RSPVPKTYDM LKAGTTATYE |
| 121 | GRWGRGTAQY SSQKSVEERS LRHPLRRLEI SPDSSPERAH |
| 161 | YTHSDYQYSQ RSQAGHTLHH QESRRAALLV PPRYARSEIV |
| 201 | GVSRAGTTSR QRHFDTYHRQ YQHGSVSDTV FDSIPANPAL |
| 241 | LTYPRPGTSR SMGNLLEKEN YLTAGLTVGQ VRPLVPLQPV |
| 281 | TQNRASRSSW HQSSFHSTRT LREAGPSVAV DSSGRRAHLT |
| 321 | VGQAAAGGSG NLLTERSTFT DSQLGNADME MTLERAVSML |
| 361 | EADHMLPSRI SAAATFIQHE CFQKSEARKR VNQLRGILKL |
| 401 | LQLLKVQNED VQRAVCGALR NLVFEDNDNK LEVAELNGVP |
| 441 | RLLQVLKQTR DLETKKQITG LLWNLSSNDK LKNLMITEAL |
| 481 | LTLTENIIIP FSGWPEGDYP KANGLLDFDI FYNVTGCLRN |
| 521 | MSSAGADGRK AMRRCDGLID SLVHYVRGTI ADYQPDDKAT |
| 561 | ENCVCILHNL SYQLEAELPE KYSQNIYIQN RNIQTDNNKS |
| 601 | IGCFGSRSRK VKEQYQDVPM PEEKSNPKGV EWLWHSIVIR |
| 641 | MYLSLIAKSV RNYTQEASLG ALQNLTAGSG PMPTSVAQTV |
| 681 | VQKESGLQHT RKMLHVGDPS VKKTAISLLR NLSRNLSLQN |
| 721 | EIAKETLPDL 7SIIPDTVPS TDLLIETTAS ACYTLNNIIQ |
| 761 | NSYQNARDLL NTGGIQKIMA ISAGDAYASN KASKAASVLL |
| 801 | YSLWAHTELH HAYKKAQFKK TDFVNSRTAK AYHSLKD |

PKP2b-881 amino acids
(SEQ ID NO: 2)

| | |
|---|---|
| 1 | MAAPGAPAEY GYIRTVLGQQ ILGQLDSSSL ALPSEAKLKL |
| 41 | AGSSGRGGQT VKSLRIQEQV QQTLARKGRS SVGNGNLHRT |
| 81 | SSVPEYVYNL HLVENDFVGG RSPVPKTYDM LKAGTTATYE |
| 121 | GRWGRGTAQY SSQKSVEERS LRHPLRRLEI SPDSSPERAH |
| 161 | YTHSDYQYSQ RSQAGHTLHH QESRRAALLV PPRYARSEIV |
| 201 | GVSRAGTTSR QRHFDTYHRQ YQHGSVSDTV FDSIPANPAL |
| 241 | LTYPRPGTSR SMGNLLEKEN YLTAGLTVGQ VRPLVPLQPV |
| 281 | TQNRASRSSW HQSSFHSTRT LREAGPSVAV DSSGRRAHLT |
| 321 | VGQAAAGGSG NLLTERSTFT DSQLGNADME MTLERAVSML |
| 361 | EADHMLPSRI SAAATFIQHE CFQKSEARKR VNQLRGILKL |
| 401 | LQLLKVQNED VQRAVCGALR NLVFEDNDNK LEVAELNGVP |
| 441 | RLLQVLKQTR DLETKKQITD HTVNLRSRNG WPGAVAHACN |
| 481 | PSTLGGQGGR ITRSGVRDQP DQHGLLWNLS SNDKLKNLMI |
| 521 | TEALLTLTEN IIIPFSGWPE GDYPKANGLL DFDIFYNVTG |
| 561 | CLRNMSSAGA DGRKAMRRCD GLIDSLVHYV RGTIADYQPD |
| 601 | DKATENCVCI LHNLSYQLEA ELPEKYSQNI YIQNRNIQTD |
| 641 | NNKSIGCFGS RSRKVKEQYQ DVPMPEEKSN PKGVEWLWHS |
| 681 | IVIRMYLSLI AKSVRNYTQE ASLGALQNLT AGSGPMPTSV |
| 721 | AQTVVQKESG LQHTRKMLHV GDPSVKKTAI SLLRNLSRNL |
| 761 | SLQNEIAKET LPDLVSIIPD TVPSTDLLIE TTASACYTLN |
| 801 | NIIQNSYQNA RDLLNTGGIQ KIMAISAGDA YASNKASKAA |
| 841 | SVLLYSLWAH TELHHAYKKA QFKKTDFVNS RTAKAYHSLK |
| 881 | D |

One experimental model of PKP2-related disease is the R735X model, as described in Cruz et al. J Am Coll Cardiol 65:1438-50 (2015). R735X is numbered according to the PKP2b isoform. The R735X mutant of PKP2a is 690 amino acids in length, due to C-terminal truncation at Arg-690, (Arg-735 relative to PKP2b, SEQ ID NO: 2).

In some embodiments, the PKP2 protein comprises a polypeptide sequence at least 75%, 80%, 85%, 90%, 92%, 93%, 94%, 95%, 96%, 97%, 98%, 99%, or 100% identical to any one of SEQ ID NO: 1 or SEQ ID NO: 2. In some embodiments, the PKP2 protein is a wild-type or native PKP2 protein, e.g. human PKP2a or human PKP2b.

In some embodiments, the disclosure provides a recombinant adeno-associated virus (rAAV) virion, comprising a capsid and a vector genome, wherein the vector genome comprises a polynucleotide sequence encoding an PKP2 or a functional variant thereof, operatively linked to a promoter. In some embodiments, the disclosure provides a recombinant adeno-associated virus (rAAV) virion, comprising a capsid and a vector genome, wherein the vector genome comprises a polynucleotide sequence encoding an PKP2, operatively linked to a promoter. The polynucleotide encoding the PKP2a may comprise a polynucleotide sequence at least 75%, 80%, 85%, 90%, 92%, 93%, 94%, 95%, 96%, 97%, 98%, 99%, or 100% identical to SEQ ID NO: 3.

The polynucleotide encoding the PKP2b may comprise a polynucleotide sequence at least 75%, 80%, 85%, 90%, 92%, 93%, 94%, 95%, 96%, 97%, 98%, 99%, or 100% identical to SEQ ID NO: 4.

Optionally, the polynucleotide sequence encoding the vector genome may comprise a Kozak sequence, including but not limited to GCCACCATGG (SEQ ID NO: 5). Kozak sequence may overlap the polynucleotide sequence encoding an PKP2a protein or a functional variant thereof. For example, the vector genome may comprise a polynucleotide sequence (with first ten nucleotides constituting the Kozak sequence) at least 75%, 80%, 85%, 90%, 92%, 93%, 94%, 95%, 96%, 97%, 98%, 99%, or 100% identical to SEQ ID NO: 6.

Kozak sequence may overlap the polynucleotide sequence encoding an PKP2b protein or a functional variant thereof. For example, the vector genome may comprise a polynucleotide sequence (with first ten nucleotides constituting the Kozak sequence) at least 75%, 80%, 85%, 90%, 92%, 93%, 94%, 95%, 96%, 97%, 98%, 99%, or 100% identical to SEQ ID NO: 7.

In some embodiments, the Kozak sequence is an alternative Kozak sequence comprising or consisting of any one of:

| (SEQ ID NO: 16) | |
|---|---|
| | (gcc)gccRccAUGG; |
| | AGNNAUGN; |
| | ANNAUGG; |
| | ANNAUGC; |
| | ACCAUGG; |
| | and |
| (SEQ ID NO: 18) | |
| | GACACCAUGG. |

In some embodiments, the vector genome comprises no Kozak sequence.

The polynucleotide sequence may be codon-optimized. For example, the vector genome may comprise a polynucleotide sequence encoding a PKP2a that shares at least 75%, 80%, 85%, 90%, 92%, 93%, 94%, 95%, 96%, 97%, 98%, 99%, or 100% identity to SEQ ID NO: 87.

The vector genome may comprise a polynucleotide sequence encoding a PKP2b that shares at least 75%, 80%, 85%, 90%, 92%, 93%, 94%, 95%, 96%, 97%, 98%, 99%, or 100% identity to SEQ ID NO: 88.

The AAV virions of the disclosure comprise a vector genome. The vector genome may comprise an expression cassette (or a polynucleotide cassette for gene-editing applications not requiring expression of the polynucleotide sequence). Any suitable inverted terminal repeats (ITRs) may be used. The ITRs may be AAV ITRs from the same serotype as the capsid present in the AAV virion, or a different serotype from the capsid (e.g., AAV2 ITRs may be used with an AAV virion having an AAV9 capsid or an AAVrh74 capsid). In each case, the serotype of the capsid determines the name applied to the virion. The ITR are generally the most 5′ and most 3′ elements of the vector genome. The vector genome will also generally contain, in 5′ to 3′ order, a promoter, a transgene, 3′ untranslated region (UTR) sequences (e.g., a WPRE element), and a polyadenylation sequence. In variations, the vector genome includes an enhancer element (generally 5′ to the promoter) and/or an exon (generally 3′ to the promoter). In variations, the vector genomes of the disclosure encode a partial or complete transgene sequence used as a repair template in a gene editing system. In such variations, the vector genome may comprise an exogenous promoter, or the gene editing system may insert the transgene into a locus in the genome having an endogenous promoter, such as a cardiac-or myocyte-specific promoter.

In some embodiments, the 5′ ITR comprises a polynucleotide sequence at least 75%, 80%, 85%, 90%, 92%, 93%, 94%, 95%, 96%, 97%, 98%, 99%, or 100% identical to SEQ ID NO: 20.

In some embodiments, the 5′ ITR comprises a polynucleotide sequence at least 75%, 80%, 85%, 90%, 92%, 93%, 94%, 95%, 96%, 97%, 98%, 99%, or 100% identical to SEQ ID NO: 21.

In some embodiments, the 5′ ITR comprises a polynucleotide sequence at least 75%, 80%, 85%, 90%, 92%, 93%, 94%, 95%, 96%, 97%, 98%, 99%, or 100% identical to SEQ ID NO: 22)

In some embodiments, the 5′ ITR comprises a polynucleotide sequence at least 75%, 80%, 85%, 90%, 92%, 93%, 94%, 95%, 96%, 97%, 98%, 99%, or 100% identical to SEQ ID NO: 23.

In some embodiments, the 3′ ITR comprises a polynucleotide sequence at least 75%, 80%, 85%, 90%, 92%, 93%, 94%, 95%, 96%, 97%, 98%, 99%, or 100% identical to SEQ ID NO: 24.

In some embodiments, the 3′ ITR comprises a polynucleotide sequence at least 75%, 80%, 85%, 90%, 92%, 93%, 94%, 95%, 96%, 97%, 98%, 99%, or 100% identical to SEQ ID NO: 25.

In some embodiments, the 3′ ITR comprises a polynucleotide sequence at least 75%, 80%, 85%, 90%, 92%, 93%, 94%, 95%, 96%, 97%, 98%, 99%, or 100% identical to SEQ ID NO: 26.

In some embodiments the vector genome comprises one or more filler sequences, e.g., at least 75%, 80%, 85%, 90%, 92%, 93%, 94%, 95%, 96%, 97%, 98%, 99%, or 100% identical to SEQ ID NO: 27; SEQ ID NO: 28; or SEQ ID NO: 29.

In some embodiments, the polynucleotide sequence encoding an PKP2 protein or functional variant thereof is operatively linked to a promoter. In certain embodiments, the promoter is an MHCK7 promoter. In certain embodiments, the promoter is an TNNT2 promoter.

The present disclosure contemplates use of various promoters. Promoters useful in embodiments of the present disclosure include, without limitation, a cytomegalovirus (CMV) promoter, phosphoglycerate kinase (PGK) promoter, or a promoter sequence comprised of the CMV enhancer and portions of the chicken beta-actin promoter and the rabbit beta-globin gene (CAG). In some cases, the promoter may be a synthetic promoter. Exemplary synthetic promoters are provided by Schlabach et al. PNAS USA. 107(6):2538-43 (2010). In some embodiments, the promoter comprises a polynucleotide sequence at least 75%, 80%, 85%, 90%, 92%, 93%, 94%, 95%, 96%, 97%, 98%, 99%, or 100% identical to SEQ ID NO: 30.

In some embodiments, a polynucleotide sequence encoding an PKP2 protein or functional variant thereof is operatively linked to an inducible promoter. An inducible promoter may be configured to cause the polynucleotide sequence to be transcriptionally expressed or not transcriptionally expressed in response to addition or accumulation of an agent or in response to removal, degradation, or dilution of an agent. The agent may be a drug. The agent may be tetracycline or one of its derivatives, including, without limitation, doxycycline. In some cases, the inducible promoter is a tet-on promoter, a tet-off promoter, a chemically-regulated promoter, a physically-regulated promoter (i.e., a promoter that responds to presence or absence of light or to low or high temperature). Inducible promoters include heavy metal ion inducible promoters (such as the mouse mammary tumor virus (mMTV) promoter or various growth hormone promoters), and the promoters from T7 phage which are active in the presence of T7 RNA polymerase. This list of inducible promoters is non-limiting.

In some cases, the promoter is a tissue-specific promoter, such as a promoter capable of driving expression in a cardiac cell to a greater extent than in a non-cardiac cell. In some embodiments, tissue-specific promoter is a selected from any various cardiac cell-specific promoters including but not limited to, desmin (Des), alpha-myosin heavy chain (α-MHC), myosin light chain 2 (MLC-2), cardiac troponin C (cTnC), cardiac troponin T (hTNNT2), muscle creatine kinase (CK) and combinations of promoter/enhancer regions thereof, such as MHCK7. In some cases, the promoter is a ubiquitous promoter. A “ubiquitous promoter” refers to a promoter that is not tissue-specific under experimental or clinical conditions. In some cases, the ubiquitous promoter is any one of Cytomegalovirus (CMV), Cytomegalovirus early enhancer element chicken beta-Actin gene intron with the splice acceptor of the rabbit beta-Globin gene (CAG), ubiquitin C (UBC), Phosphoglycerate Kinase (PGK), Eukaryotic translation elongation factor 1 alpha 1 (EF1-alpha), Glyceraldehyde 3-phosphate dehydrogenase (GAPDH), simian virus 40 (SV40), Hepatitis B virus (HBV), chicken beta-actin, and human beta-actin promoters.

In some embodiments, the promoter sequence is selected from Table 3. In some embodiments, the promoter comprises a polynucleotide sequence at least 75%, 80%, 85%, 90%, 92%, 93%, 94%, 95%, 96%, 97%, 98%, 99%, or 100% identical to any one of SEQ ID NOS: 31-51. In some embodiments, the promoter comprises a fragment of a polynucleotide sequence of any one of SEQ ID NOs: 31-51, e.g., a fragment comprising at least 25%, at least 50%, at least 75%, 80%, 85%, 90%, 92%, 93%, 94%, 95%, 96%, 97%, 98%, 99%, or 100% of any one of SEQ ID NOs: 31-51.

TABLE 3

| | | SEQ ID |
|---|---|---|
| PROMOTER | SEQUENCE | NO: |
| MHCK7 | ACCCTTCAGATTAAAAATAACTGAGGTAAGGGCCTGGGTAGG | 31 |
| | GGAGGTGGTGTGAGACGCTCCTGTCTCTCCTCTATCTGCCCA | |
| | TCGGCCCTTTGGGGAGGAGGAATGTGCCCAAGGACTAAAAAA | |
| | AGGCCATGGAGCCAGAGGGGCGAGGGCAACAGACCTTTCATG | |
| | GGCAAACCTTGGGGCCCTGCTGTCTAGCATGCCCCACTACGG | |
| | GTCTAGGCTGCCCATGTAAGGAGGCAAGGCCTGGGGACACCC | |
| | GAGATGCCTGGTTATAATTAACCCAGACATGTGGCTGCCCCC | |
| | CCCCCCCCAACACCTGCTGCCTCTAAAAATAACCCTGTCCCT | |
| | GGTGGATCCCCTGCATGCGAAGATCTTCGAACAAGGCTGTGG | |
| | GGGACTGAGGGCAGGCTGTAACAGGCTTGGGGGCCAGGGCTT | |
| | ATACGTGCCTGGGACTCCCAAAGTATTACTGTTCCATGTTCC | |
| | CGGCGAAGGGCCAGCTGTCCCCCGCCAGCTAGACTCAGCACT | |
| | TAGTTTAGGAACCAGTGAGCAAGTCAGCCCTTGGGGCAGCCC | |
| | ATACAAGGCCATGGGGCTGGGCAAGCTGCACGCCTGGGTCCG | |
| | GGGTGGGCACGGTGCCCGGGCAACGAGCTGAAAGCTCATCTG | |
| | CTCTCAGGGGCCCCTCCCTGGGGACAGCCCCTCCTGGCTAGT | |
| | CACACCCTGTAGGCTCCTCTATATAACCCAGGGGCACAGGGG | |
| | CTGCCCTCATTCTACCACCACCTCCACAGCACAGACAGACAC | |
| | TCAGGAGCCAGCCAG | |
| Human cardiac | CTCAGTCCATTAGGAGCCAGTAGCCTGGAAGATGTCTTTACC | 33 |
| troponin T | CCCAGCATCAGTTCAAGTGGAGCAGCACATAACTCTTGCCCT | |
| promoter | CTGCCTTCCAAGATTCTGGTGCTGAGACTTATGGAGTGTCTT | |
| (without | GGAGGTTGCCTTCTGCCCCCCAACCCTGCTCCCAGCTGGCCC | |
| exon 1) | TCCCAGGCCTGGGTTGCTGGCCTCTGCTTTATCAGGATTCTC | |
| hTnnT2/ | AAGAGGGACAGCTGGTTTATGTTGCATGACTGTTCCCTGCAT | |
| HTNNT2 | ATCTGCTCTGGTTTTAAATAGCTTATCTGAGCAGCTGGAGGA | |
| | CCACATGGGCTTATATGGCGTGGGGTACATGTTCCTGTAGCC | |
| | TTGTCCCTGGCACCTGCCAAAATAGCAGCCAACACCCCCCAC | |
| | CCCCACCGCCATCCCCCTGCCCCACCCGTCCCCTGTCGCACA | |
| | TTCCTCCCTCCGCAGGGCTGGCTCACCAGGCCCCAGCCCACA | |
| | TGCCTGCTTAAAGCCCTCTCCATCCTCTGCCTCACCCAGT | |
| Human cardiac | CTCAGTCCATTAGGAGCCAGTAGCCTGGAAGATGTCTTTACC | 32 |
| troponin T | CCCAGCATCAGTTCAAGTGGAGCAGCACATAACTCTTGCCCT | |
| promoter (with | CTGCCTTCCAAGATTCTGGTGCTGAGACTTATGGAGTGTCTT | |
| exon 1, | GGAGGTTGCCTTCTGCCCCCCAACCCTGCTCCCAGCTGGCCC | |
| underlined) | TCCCAGGCCTGGGTTGCTGGCCTCTGCTTTATCAGGATTCTC | |
| hTnnT2/ | AAGAGGGACAGCTGGTTTATGTTGCATGACTGTTCCCTGCAT | |
| HTNNT2 | ATCTGCTCTGGTTTTAAATAGCTTATCTGAGCAGCTGGAGGA | |
| | CCACATGGGCTTATATGGCGTGGGGTACATGTTCCTGTAGCC | |
| | TTGTCCCTGGCACCTGCCAAAATAGCAGCCAACACCCCCCAC | |
| | CCCCACCGCCATCCCCCTGCCCCACCCGTCCCCTGTCGCACA | |
| | TTCCTCCCTCCGCAGGGCTGGCTCACCAGGCCCCAGCCCACA | |
| | TGCCTGCTTAAAGCCCTCTCCATCCTCTGCCTCACCCAGTCC | |
| | CCGCTGAGACTGAGCAGACGCCTCCAGGATCTGTCGGCAG | |
| Mouse α- | GGTACCGGATCCTGCAAGGTCACACAAGGGTCTCCACCCACC | 34 |
| cardiac myosin | AGGTGCCCTAGTCTCAATTTCAGTTTCCATGCCTTGTTCTCA | |
| heavy chain | CAATGCTGGCCTCCCCAGAGCTAATTTGGACTTTGTTTTTAT | |
| promoter | TTCAAAAGGGCCTGAATGAGGAGTAGATCTTGTGCTACCCAG | |
| (αMHC) | CTCTAAGGGTGCCCGTGAAGCCCTCAGACCTGGAGCCTTTGC | |
| | AACAGCCCTTTAGGTGGAAGCAGAATAAAGCAATTTTCCTTA | |
| | AAGCCAAAATCCTGCCTCTAGACTCTTCTTCTCTGACCTCGG | |
| | TCCCTGGGCTCTAGGGTGGGGAGGTGGGGCTTGGAAGAAGAA | |
| | GGTGGGGAAGTGGCAAAAGCCGATCCCTAGGGCCCTGTGAAG | |
| | TTCGGAGCCTTCCCTGTACAGCACTGGCTCATAGATCCTCCT | |
| | CCAGCCAAACATAGCAAGAAGTGATACCTCCTTTGTGACTTC | |
| | CCCAGGCCCAGTACCTGTCAGGTTGAAACAGGATTTAGAGAA | |
| | GCCTCTGAACTCACCTGAACTCTGAAGCTCATCCACCAAGCA | |
| | AGCACCTAGGTGCCACTGCTAGTTAGTATCCTACGCTGATAA | |
| | TATGCAGAGCTGGGCCACAGAAGTCCTGGGGTGTAGGAACTG | |
| | ACCAGTGACTTTTCAGTCGGCAAAGGTATGACCCCCTCAGCA | |
| | GATGTAGTAATGTCCCCTTAGATCCCATCCCAGGCAGGTCTC | |
| | TAAGAGGACATGGGATGAGAGATGTAGTCATGTGGCATTCCA | |
| | AACACAGCTATCCACAGTGTCCCTTGCCCCTTCCACTTAGCC | |
| | AGGAGGACAGTAACCTTAGCCTATCTTTCTTCCTCCCCATCC | |
| | TCCCAGGACACACCCCCTGGTCTGCAGTATTCATTTCTTCCT | |
| | TCACGTCCCCTCTGTGACTTCCATTTGCAAGGCTTTTGACCT | |
| | CTGCAGCTGCTGGAAGATAGAGTTTGGCCCTAGGTGTGGCAA | |
| | GCCATCTCAAGAGAAAGCAGACAACAGGGGGACCAGATTTTG | |
| | GAAGGATCAGGAACTAAATCACTGGCGGGCCTGGGGGTAGAA | |
| | AAAAGAGTGAGTGAGTCCGCTCCAGCTAAGCCAAGCTAGTCC | |
| | CCGAGATACTCTGCCACAGCTGGGCTGCTCGGGGTAGCTTTA | |
| | GGAATGTGGGTCTGAAAGACAATGGGATTGGAAGACATCTCT | |
| | TTGAGTCTCCCCTCAACCCCACCTACAGACACACTCGTGTGT | |
| | GGCCAGACTCCTGTTCAACAGCCCTCTGTGTTCTGACCACTG | |
| | AGCTAGGCAACCAGAGCATGGGCCCTGTGCTGAGGATGAAGA | |
| | GTTGGTTACCAATAGCAAAAACAGCAGGGGAGGGAGAACAGA | |
| | GAACGAAATAAGGAAGGAAGAAGGAAAGGCCAGTCAATCAGA | |
| | TGCAGTCAGAAGAGATGGGAAGCCAACACACAGCTTGAGCAG | |
| | AGGAAACAGAAAAGGGAGAGATTCTGGGCATAAGGAGGCCAC | |
| | AGAAAGAAGAGCCCAGGCCCCCCAAGTCTCCTCTTTATACCC | |
| | TCATCCCGTCTCCCAATTAAGCCCACTCTTCTTCCTAGATCA | |
| | GACCTGAGCTGCAGCGAAGAGACCCGTAGGGAGGATCACACT | |
| | GGATGAAGGAGATGTGTGGAGAAGTCCAGGGAACCTAAGAGC | |
| | CAGAGCCTAAAAGAGCAAGAGATAAAGGTGCTTCAAAGGTGG | |
| | CCAGGCTGTGCACACAGAGGGTCGAGGACTGGTGGTAGAGCC | |
| | TCAAGATAAGGATGATGCTCAGAATGGGCGGGGGGGGGGATT | |
| | CTGGGGGGGGGAGAGAGAAGGTGAGAAGGAGCCTGGAACAGA | |
| | GAATCTGGAAGCGCTGGAAACGATACCATAAAGGGAAGAACC | |
| | CAGGCTACCTTTAGATGTAAATCATGAAAGACAGGGAGAAGG | |
| | GAAGCTGGAGAGAGTAGAAGGACCCCGGGGCAAGACATTGAA | |
| | GCAAGGACAAGCCAGGTTGAGCGCTCCGTGAAATCAGCCTGC | |
| | TGAAGGCAGAGCCCTGGTATGAGCACCAGAACAGCAGAGGCT | |
| | AGGGTTAATGTCGAGACAGGGAACAGAAGGTAGACACAGGAA | |
| | CAGACAGAGACGGGGGAGCCAGGTAACAAAGGAATGGTCCTT | |
| | CTCACCTGTGGCCAGAGCGTCCATCTGTGTCCACATACTCTA | |
| | GAATGTTCATCAGACTGCAGGGCTGGCTTGGGAGGCAGCTGG | |
| | AAAGAGTATGTGAGAGCCAGGGGAGACAAGGGGGCCTAGGAA | |
| | AGGAAGAAGAGGGCAAACCAGGCCACACAAGAGGGCAGAGCC | |
| | CAGAACTGAGTTAACTCCTTCCTTGTTGCATCTTCCATAGGA | |
| | GGCAGTGGGAACTCTGTGACCACCATCCCCCATGAGCCCCCA | |
| | CTACCCATACCAAGTTTGGCCTGAGTGGCATTCTAGGTTCCC | |
| | TGAGGACAGAGCCTGGCCTTTGTCTCTTGGACCTGACCCAAG | |
| | CTGACCCAATGTTCTCAGTACCTTATCATGCCCTCAAGAGCT | |
| | TGAGAACCAGGCAGTGACATATTAGGCCATGGGCTAACCCTG | |
| | GAGCTTGCACACAGGAGCCTCAAGTGACCTCCAGGGACACAG | |
| | CTGCAGACAGGTGGCCTTTATCCCCAAAGAGCAACCATTTGG | |
| | CATAGGTGGCTGCAAATGGGAATGCAAGGTTGAATCAGGTCC | |
| | CTTCAAGAATACTGCATGCAAGACCTAAGACCCCTGGAGAGA | |
| | GGGGTATGCTCCTGCCCCCACCCACCATAAGGGGAGTGAACT | |
| | ATCCTAGGGGGCTGGCGACCTTGGGGAGACACCACATTACTG | |
| | AGAGTGCTGAGCCCAGAAAAACTGACCGCCCTGTGTCCTGCC | |
| | CACCTCCACACTCTAGAGCTATATTGAGAGGTGACAGTAGAT | |
| | AGGGTGGGAGCTGGTAGCAGGGAGAGTGTTCCTGGGTGTGAG | |
| | GGTGTAGGGGAAAGCCAGAGCAGGGGAGTCTGGCTTTGTCTC | |
| | CTGAACACAATGTCTACTTAGTTATAACAGGCATGACCTGCT | |
| | AAAGACCCAACATCTACGACCTCTGAAAAGACAGCAGCCCTG | |
| | GAGGACAGGGGTTGTCTCTGAGCCTTGGGTGCTTGATGGTGC | |
| | CACAAAGGAGGGCATGAGTGTGAGTATAAGGCCCCAGGAGCG | |
| | TTAGAGAAGGGCACTTGGGAAGGGGTCAGTCTGCAGAGCCCC | |
| | TATCCATGGAATCTGGAGCCTGGGGCCAACTGGTGTAAATCT | |
| | CTGGGCCTGCCAGGCATTCAAAGCAGCACCTGCATCCTCTGG | |
| | CAGCCTGGGGAGGCGGAAGGGAGCAACCCCCCACTTATACCC | |
| | TTTCTCCCTCAGCCCCAGGATTAACACCTCTGGCCTTCCCCC | |
| | TTCCCACCTCCCATCAGGAGTGGAGGGTTGCAGAGGGAGGGT | |
| | AAAAACCTACATGTCCAAACATCATGGTGCACGATATATGGA | |
| | TCAGTATGTGTAGAGGCAAGAAAGGAAATCTGCAGGCTTAAC | |
| | TGGGTTAATGTGTAAAGTCTGTGTGCATGTGTGTGTGTCTGA | |
| | CTGAAAACGGGCATGGCTGTGCAGCTGTTCAGTTCTGTGCGT | |
| | GAGGTTACCAGACTGCAGGTTTGTGTGTAAATTGCCCAAGGC | |
| | AAAGTGGGTGAATCCCTTCCATGGTTTAAAGAGATTGGATGA | |
| | TGGCCTGCATCTCAAGGACCATGGAAAATAGAATGGACACTC | |
| | TATATGTGTCTCTAAGCTAAGGTAGCAAGGTCTTTGGAGGAC | |
| | ACCTGTCTAGAGATGTGGGCAACAGAGACTACAGACAGTATC | |
| | TGTACAGAGTAAGGAGAGAGAGGAGGGGGTGTAGAATTCTCT | |
| | TACTATCAAAGGGAAACTGAGTCGTGCACCTGCAAAGTGGAT | |
| | GCTCTCCCTAGACATCATGACTTTGTCTCTGGGGAGCCAGCA | |
| | CTGTGGAACTTCAGGTCTGAGAGAGTAGGAGGCTCCCCTCAG | |
| | CCTGAAGCTATGCAGATAGCCAGGGTTGAAAGGGGGAAGGGA | |
| | GAGCCTGGGATGGGAGCTTGTGTGTTGGAGGCAGGGGACAGA | |
| | TATTAAGCCTGGAAGAGAAGGTGACCCTTACCCAGTTGTTCA | |
| | ACTCACCCTTCAGATTAAAAATAACTGAGGTAAGGGCCTGGG | |
| | TAGGGGAGGTGGTGTGAGACGCTCCTGTCTCTCCTCTATCTG | |
| | CCCATCGGCCCTTTGGGGAGGAGGAATGTGCCCAAGGACTAA | |
| | AAAAAGGCCATGGAGCCAGAGGGGCGAGGGCAACAGACCTTT | |
| | CATGGGCAAACCTTGGGGCCCTGCTGTCCTCCTGTCACCTCC | |
| | AGAGCCAAGGGATCAAAGGAGGAGGAGCCAGGACAGGAGGGA | |
| | AGTGGGAGGGAGGGTCCCAGCAGAGGACTCCAAATTTAGGCA | |
| | GCAGGCATATGGGATGGGATATAAAGGGGCTGGAGCACTGAG | |
| | AGCTGTCAGAGATTTCTCCAACCCAGGTAAGAGGGAGTTTCG | |
| | GGTGGGGGCTCTTCACCCACACCAGACCTCTCCCCACCTAGA | |
| | AGGAAACTGCCTTTCCTGGAAGTGGGGTTCAGGCCGGTCAGA | |
| | GATCTGACAGGGTGGCCTTCCACCAGCCTGGGAAGTTCTCAG | |
| | TGGCAGGAGGTTTCCACAAGAAACACTGGATGCCCCTTCCCT | |
| | TACGCTGTCTTCTCCATCTTCCTCCTGGGGATGCTCCTCCCC | |
| | GTCTTGGTTTATCTTGGCTCTTCGTCTTCAGCAAGATTTGCC | |
| | CTGTGCTGTCCACTCCATCTTTCTCTACTGTCTCCGTGCCTT | |
| | GCCTTGCCTTCTTGCGTGTCCTTCCTTTCCACCCATTTCTCA | |
| | CTTCACCTTTTCTCCCCTTCTCATTTGTATTCATCCTTCCTT | |
| | CCTTCCTTCCTTCCTTCCTTCCTTCCTTCCTTCCTTCCTTTC | |
| | TCCCTTCCTTCCTTCCTTCCTTCCTTCCTTCCTTCCTTCCTT | |
| | CCTGTGTCAGAGTGCTGAGAATCACACCTGGGGTTCCCACCC | |
| | TTATGTAAACAATCTTCCAGTGAGCCACAGCTTCAGTGCTGC | |
| | TGGGTGCTCTCTTACCTTCCTCACCCCCTGGCTTGTCCTGTT | |
| | CCATCCTGGTCAGGATCTCTAGATTGGTCTCCCAGCCTCTGC | |
| | TACTCCTCTTCCTGCCTGTTCCTCTCTCTGTCCAGCTGCGCC | |
| | ACTGTGGTGCCTCGTTCCAGCTGTGGTCCACATTCTTCAGGA | |
| | TTCTCTGAAAAGTTAACCAGGTGAGAATGTTTCCCCTGTAGA | |
| | CAGCAGATCACGATTCTCCCGGAAGTCAGGCTTCCAGCCCTC | |
| | TCTTTCTCTGCCCAGCTGCCCGGCACTCTTAGCAAACCTCAG | |
| | GCACCCTTACCCCACATAGACCTCTGACAGAGAAGCAGGCAC | |
| | TTTACATGGAGTCCTGGTGGGAGAGCCATAGGCTACGGTGTA | |
| | AAAGAGGCAGGGAAGTGGTGGTGTAGGAAAGTCAGGACTTCA | |
| | CATAGAAGCCTAGCCCACACCAGAAATGACAGACAGATCCCT | |
| | CCTATCTCCCCCATAAGAGTTTGAGTCGACCCGCGGCCCCGA | |
| | ATTG | |
| Chicken cardiac | GGGATAAAAGCAGTCTGGGCTTTCACATGACAGCATCTGGGG | 35 |
| troponin T | CTGCGGCAGAGGGTCGGGTCCGAAGCGCTGCCTTATCAGCGT | |
| promoter | CCCCAGCCCTGGGAGGTGACAGCTGGCTGGCTTGTGTCAGCC | |
| (cTnT) | CCTCGGGCACTCACGTATCTCCGTCCGACGGGTTTAAAATAG | |
| | CAAAACTCTGAGGCCACACAATAGCTTGGGCTTATATGGGCT | |
| | CCTGTGGGGGAAGGGGGAGCACGGAGGGGGCCGGGGCCGCTG | |
| | CTGCCAAAATAGCAGCTCACAAGTGTTGCATTCCTCTCTGGG | |
| | CGCCGGGCACATTCCTGCTGGCTCTGCCCGCCCCGGGGTGGG | |
| | CGCCGGGGGGACCTTAAAGCCTCTGCCCCCCAAGGAGCCCTT | |
| | CCCAGACAGCCGCCGGCACCCACCGCTCCGTGGGA | |
| Human Creatine | CTCTCAGCCCTGGAAGTCCTTGCTCACAGCCGAGGCGCCGAG | 36 |
| Kinase M | AGCGCTTGCTCTGCCCAGATCTGCGCGAGTCTGGCGCCCGCG | |
| (hCKM) | CTCTGAACGGCGTCGCTGCCCAGCCCCCTTCCCCGGGAGGTG | |
| | GGAGCGGCCACCCAGGGCCCCGTGGCTGCCCTTGTAAGGAGG | |
| | CGAGGCCCGAGGACACCCGAGACGCCCGGTTATAATTAACCA | |
| | GGACACGTGGCGAACCCCCCTCCAACACCTGCCCCCGAACCC | |
| | CCCCATACCCAGCGCCTCGGGTCTCGGCCTTTGCGGCAGAGG | |
| | AGACAGCAAAGCGCCCTCTAAAAATAACTCCTTTCCCGGCGA | |
| | CCGAGACCCTCCCTGTCCCCCGCACAGCGGAAATCTCCCAGT | |
| | GGCACCGAGGGGGCGAGGGTTAAGTGGGGGGGAGGGTGACCA | |
| | CCGCCTCCCACCCTTGCCCTGAGTTTGAATCTCTCCAACTCA | |
| | GCCAGCCTCAGTTTCCCCTCCACTCAGTCCCTAGGAGGAAGG | |
| | GGCGCCCAAGCGCGGGTTTCTGGGGTTAGACTGCCCTCCATT | |
| | GCAATTGGTCCTTCTCCCGGCCTCTGCTTCCTCCAGCTCACA | |
| | GGGTATCTGCTCCTCCTGGAGCCACACCTTGGTTCCCCGAGG | |
| | TGCCGCTGGGACTCGGGTAGGGGTGAGGGCCCAGGGGGCACA | |
| | GGGGGAGCCGAGGGCCACAGGAAGGGCTGGTGGCTGAAGGAG | |
| | ACTCAGGGGCCAGGGGACGGTGGCTTCTACGTGCTTGGGACG | |
| | TTCCCAGCCACCGTCCCATGTTCCCGGCGGGGGGCCAGCTGT | |
| | CCCCACCGCCAGCCCAACTCAGCACTTGGTCAGGGTATCAGC | |
| | TTGGTGGGGGGGCGTGAGCCCAGCCCCTGGGGCGGCTCAGCC | |
| | CATACAAGGCCATGGGGCTGGGCGCAAAGCATGCCTGGGTTC | |
| | AGGGTGGGTATGGTGCGGGAGCAGGGAGGTGAGAGGCTCAGC | |
| | TGCCCTCCAGAACTCCTCCCTGGGGACAACCCCTCCCAGCCA | |
| | ATAGCACAGCCTAGGTCCCCCTATATAAGGCCACGGCTGCTG | |
| | GCCCTTCCTTTGGGTCAGTGTCACCTCCAGGATACAGACA | |
| Human beta- | GCCCAGCACCCCAAGGCGGCCAACGCCAAAACTCTCCCTCCT | 37 |
| actin (HuBa) | CCTCTTCCTCAATCTCGCTCTCGCTCTTTTTTTTTTTCGCAA | |
| | AAGGAGGGGAGAGGGGGTAAAAAAATGCTGCACTGTGCGGCG | |
| | AAGCCGGTGAGTGAGCGGCGCGGGGCCAATCAGCGTGCGCCG | |
| | TTCCGAAAGTTGCCTTTTATGGCTCGAGCGGCCGCGGCGGCG | |
| | CCCTATAAAACCCAGCGGCGCGACGCGCCACCACCGCCGAGT | |
| | C | |
| Chicken beta- | GGTCGAGGTGAGCCCCACGTTCTGCTTCACTCTCCCCATCTC | 38 |
| actin (CBA) | CCCCCCCTCCCCACCCCCAATTTTGTATTTATTTATTTTTTA | |
| | GGTCGAGGTGAGCCCCACGTTCTGCTTCACTCTCCCCATCTC | |
| | CCCCCCCTCCCCACCCCCAATTTTGTATTTATTTATTTTTTA | |
| | ATTATTTTGTGCAGCGATGGGGGCGGGGGGGGGGGGGGCGCG | |
| | CGCCAGGCGGGGCGGGGCGGGGCGAGGGGCGGGGCGGGGCGA | |
| | GGCGGAGAGGTGCGGCGGCAGCCAATCAGAGCGGCGCGCTCC | |
| | GAAAGTTTCCTTTTATGGCGAGGCGGCGGCGGCGGCGGCCCT | |
| | ATAAAAAGCGAAGCGCGCGGCGGGCGGGA | |
| Cytomegalovirus | TGGTGATGCGGTTTTGGCAGTACACCAATGGGCGTGGATAGC | 39 |
| (CMV) | GGTTTGACTCACGGGGATTTCCAAGTCTCCACCCCATTGACG | |
| | TGGTGATGCGGTTTTGGCAGTACACCAATGGGCGTGGATAGC | |
| | GGTTTGACTCACGGGGATTTCCAAGTCTCCACCCCATTGACG | |
| | TCAATGGGAGTTTGTTTTGGCACCAAAATCAACGGGACTTTC | |
| | CAAAATGTCGTAATAACCCCGCCCCGTTGACGCAAATGGGCG | |
| | GTAGGCGTGTACGGTGGGAGGTCTATATAAGCAGAGCTCGTT | |
| | TAGTGAACCG | |
| Cytomegalovirus | TAGTTATTAATAGTAATCAATTACGGGGTCATTAGTTCATAG | 40 |
| (CMV) | CCCATATATGGAGTTCCGCGTTACATAACTTACGGTAAATGG | |
| (second | CCCGCCTGGCTGACCGCCCAACGACCCCCGCCCATTGACGTC | |
| version) | AATAATGACGTATGTTCCCATAGTAACGCCAATAGGGACTTT | |
| | CCATTGACGTCAATGGGTGGAGTATTTACGGTAAACTGCCCA | |
| | CTTGGCAGTACATCAAGTGTATCATATGCCAAGTACGCCCCC | |
| | TATTGACGTCAATGACGGTAAATGGCCCGCCTGGCATTATGC | |
| | CCAGTACATGACCTTATGGGACTTTCCTACTTGGCAGTACAT | |
| | CTACGTATTAGTCATCGCTATTACCATGGTGATGCGGTTTTG | |
| | GCAGTACATCAATGGGCGTGGATAGCGGTTTGACTCACGGGG | |
| | ATTTCCAAGTCTCCACCCCATTGACGTCAATGGGAGTTTGTT | |
| | TTGGCACCAAAATCAACGGGACTTTCCAAAATGTCGTAACAA | |
| | CTCCGCCCCATTGACGCAAATGGGCGGTAGGCGTGTACGGTG | |
| | GGAGGTCTATATAAGCAGAGCTGGTTTAGTGAACCGT | |
| Cytomegalovirus | CGTTACATAACTTACGGTAAATGGCCCGCCTGGCTGACCGCC | 41 |
| (CMV) (third | CAACGACCCCCGCCCATTGACGTCAATAATGACGTATGTTCC | |
| version) | CATAGTAACGCCAATAGGGACTTTCCATTGACGTCAATGGGT | |
| | GGAGTATTTACGGTAAACTGCCCACTTGGCAGTACATCAAGT | |
| | GTATCATATGCCAAGTACGCCCCCTATTGACGTCAATGACGG | |
| | TAAATGGCCCGCCTGGCATTATGCCCAGTACATGACCTTATG | |
| | GGACTTTCCTACTTGGCAGTACATCTACGTATTAGTCATCGC | |
| | TATTACCATGGTGATGCGGTTTTGGCAGTACATCAATGGGCG | |
| | TGGATAGCGGTTTGACTCACGGGGATTTCCAAGTCTCCACCC | |
| | CATTGACGTCAATGGGAGTTTGTTTTGGCACCAAAATCAACG | |
| | GGACTTTCCAAAATGTCGTAACAACTCCGCCCCATTGACGCA | |
| | AATGGGCGGTAGGCGTGTACGGTGGGAGGTCTATATAAGCAG | |
| | AGCT | |
| CAG promoter | ACTTACGGTAAATGGCCCGCCTGGCTGACCGCCCAACGACCC | 42 |
| (first version) | CCGCCCATTGACGTCAATAATGACGTATGTTCCCATAGTAAC | |
| | GCCAATAGGGACTTTCCATTGACGTCAATGGGTGGAGTATTT | |
| | ACGGTAAACTGCCCACTTGGCAGTACATCAAGTGTATCATAT | |
| | GCCAAGTACGCCCCCTATTGACGTCAATGACGGTAAATGGCC | |
| | CGCCTGGCATTATGCCCAGTACATGACCTTATGGGACTTTCC | |
| | TACTTGGCAGTACATCTACGTATTAGTCATCGCTATTACCAT | |
| | GGTCGAGGTGAGCCCCACGTTCTGCTTCACTCTCCCCATCTC | |
| | CCCCCCCTCCCCACCCCCAATTTTGTATTTATTTATTTTTTA | |
| | ATTATTTTGTGCAGCGATGGGGGCGGGGGGGGGGGGGGCGCG | |
| | CGCCAGGCGGGGCGGGGCGGGGCGAGGGGCGGGGCGGGGCGA | |
| | GGCGGAGAGGTGCGGCGGCAGCCAATCAGAGCGGCGCGCTCC | |
| | GAAAGTTTCCTTTTATGGCGAGGCGGCGGCGGCGGCGGCCCT | |
| | ATAAAAAGCGAAGCGCGCGGCGGGCGG | |
| CAG promoter | CGTTACATAACTTACGGTAAATGGCCCGCCTGGCTGACCGCC | 43 |
| (second | CAACGACCCCCGCCCATTGACGTCAATAATGACGTATGTTCC | |
| version) | CATAGTAACGCCAATAGGGACTTTCCATTGACGTCAATGGGT | |
| | GGAGTATTTACGGTAAACTGCCCACTTGGCAGTACATCAAGT | |
| | GTATCATATGCCAAGTACGCCCCCTATTGACGTCAATGACGG | |
| | TAAATGGCCCGCCTGGCATTATGCCCAGTACATGACCTTATG | |
| | GGACTTTCCTACTTGGCAGTACATCTACGTATTAGTCATCGC | |
| | TATTACCATGTCGAGGTGAGCCCCACGTTCTGCTTCACTCTC | |
| | CCCATCTCCCCCCCCTCCCCACCCCCAATTTTGTATTTATTT | |
| | ATTTTTTAATTATTTTGTGCAGCGATGGGGGCGGGGGGGGGG | |
| | GGGGCGCGCGCCAGGCGGGGCGGGGCGGGGCGAGGGGCGGGG | |
| | CGGGGCGAGGCGGAGAGGTGCGGCGGCAGCCAATCAGAGCGG | |
| | CGCGCTCCGAAAGTTTCCTTTTATGGCGAGGCGGCGGCGGCG | |
| | GCGGCCCTATAAAAAGCGAAGCGCGCGGCGGGCG | |
| Human EF1- | CAACCTTTGGAGCTAAGCCAGCAATGGTAGAGGGAAGATTCT | 44 |
| alpha (EF1-α) | GCACGTCCCTTCCAGGCGGCCTCCCCGTCACCACCCCCCCCA | |
| | ACCCGCCCCGACCGGAGCTGAGAGTAATTCATACAAAAGGAC | |
| | TCGCCCCTGCCTTGGGGAATCCCAGGGACCGTCGTTAAACTC | |
| | CCACTAACGTAGAACCCAGAGATCGCTGCGTTCCCGCCCCCT | |
| | CACCCGCCCGCTCTCGTCATCACTGAGGTGGAGAATAGCATG | |
| | CGTGAGGCTCCGGTGCCCGTCAGTGGGCAGAGCGCACATCGC | |
| | CCACAGTCCCCGAGAAGTTGGGGGGAGGGGTCGGCAATTGAA | |
| | CGGGTGCCTAGAGAAGGTGGCGCGGGGTAAACTGGGAAAGTG | |
| | ATGTCGTGTACTGGCTCCGCCTTTTTCCCGAGGGTGGGGGAG | |
| | AACCGTATATAAGTGCAGTAGTCGCCGTGAACGTT | |
| Human | ACTTGTGGACAAAGTTTGCTCTATTCCACCTCCTCCAGGCCC | 48 |
| CamKIIa | TCCTTGGGTCCATCACCCCAGGGGTGCTGGGTCCATCCCACC | |
| (CaMKIIa) | CCCAGGCCCACACAGGCTTGCAGTATTGTGTGCGGTATGGTC | |
| | AGGGCGTCCGAGAGCAGGTTTCGCAGTGGAAGGCAGGCAGGT | |
| | GTTGGGGAGGCAGTTACCGGGGCAACGGGAACAGGGCGTTTT | |
| | GGAGGTGGTTGCCATGGGGACCTGGATGCTGACGAAGGCTCG | |
| | CGAGGCTGTGAGCAGCCACAGTGCCCTGC | |

In a certain embodiment, the vector genome comprises a polynucleotide sequence at least 75%, 80%, 85%, 90%, 92%, 93%, 94%, 95%, 96%, 97%, 98%, 99%, or 100% identical to SEQ ID NO: 31. In a certain embodiment, the vector genome comprises a polynucleotide sequence at least 75%, 80%, 85%, 90%, 92%, 93%, 94%, 95%, 96%, 97%, 98%, 99%, or 100% identical to SEQ ID NO: 32. In a certain embodiment, the vector genome comprises a polynucleotide sequence at least 75%, 80%, 85%, 90%, 92%, 93%, 94%, 95%, 96%, 97%, 98%, 99%, or 100% identical to SEQ ID NO: 33.

Further illustrative examples of promoters are the SV40 late promoter from simian virus 40, the Baculovirus polyhedron enhancer/promoter element, Herpes Simplex Virus thymidine kinase (HSV tk), the immediate early promoter from cytomegalovirus (CMV) and various retroviral promoters including LTR elements. A large variety of other promoters are known and generally available in the art, and the sequences of many such promoters are available in sequence databases such as the GenBank database.

In some cases, vectors of the present disclosure further comprise one or more regulatory elements selected from the group consisting of an enhancer, an intron, a poly-A signal, a 2A peptide encoding sequence, a WPRE (Woodchuck hepatitis virus posttranscriptional regulatory element), and a HPRE (Hepatitis B posttranscriptional regulatory element).

In some embodiments, the vector comprises a CMV enhancer.

In certain embodiments, the vectors comprise one or more enhancers. In particular embodiments, the enhancer is a CMV enhancer sequence, a GAPDH enhancer sequence, a β-actin enhancer sequence, or an EF1-α enhancer sequence. Sequences of the foregoing are known in the art. For example, the sequence of the CMV immediate early (IE) enhancer is SEQ ID NO: 50.

In certain embodiments, the vectors comprise one or more introns. In particular embodiments, the intron is a rabbit globin intron sequence, a chicken β-actin intron sequence, a synthetic intron sequence, an SV40 intron, or an EF1-α intron sequence.

In certain embodiments, the vectors comprise a poly A sequence. In particular embodiments, the polyA sequence is a rabbit globin polyA sequence, a human growth hormone polyA sequence, a bovine growth hormone poly A sequence, a PGK polyA sequence, an SV40 polyA sequence, or a TK polyA sequence. In some embodiments, the poly-A signal may be a bovine growth hormone polyadenylation signal (bGHpA).

In certain embodiments, the vectors comprise one or more transcript stabilizing element. In particular embodiments, the transcript stabilizing element is a WPRE sequence, a HPRE sequence, a scaffold-attachment region, a 3′ UTR, or a 5′ UTR. In particular embodiments, the vectors comprise both a 5′ UTR and a 3′ UTR.

In some embodiments, the vector comprises a 5′ untranslated region (UTR) selected from Table 4. In some embodiments, the vector genome comprises a polynucleotide sequence at least 75%, 80%, 85%, 90%, 92%, 93%, 94%, 95%, 96%, 97%, 98%, 99%, or 100% identical to any one of SEQ ID NOS 51-61.

TABLE 4

| 5′ | | SEQ |
|---|---|---|
| UNTRANSLATED | | ID |
| REGION | SEQUENCE | NO: |
| Human beta-actin | CGCGTCCGCCCGCGAGCACAGAGCCTCGCCTTTGCCGATC | 51 |
| exon/intron | CGCCGCCCGTCCACACCCGCCGCCAGGTAAGCCCGGCCAG | |
| | CCGACCGGGGCATGCGGCCGCGGCCCTTCGCCCGTGCAGA | |
| | GCCGCCGTCTGGGCCGCAGCGGGGGGCGCATGGGGCGGAA | |
| | CCGGACCGCCGTGGGGGGCGCGGGAGAAGCCCCTGGGCCT | |
| | CCGGAGATGGGGGACACCCCACGCCAGTTCGCAGGCGCGA | |
| | GGCCGCGCTCGGGCGGGCGCGCTCCGGGGGTGCCGCTCTC | |
| | GGGGCGGGGGCAACCGGCGGGGTCTTTGTCTGAGCCGGGC | |
| | TCTTGCCAATGGGGATCGCACGGTGGGCGCGGCGTAGCCC | |
| | CCGTCAGGCCCGGTGGGGGCTGGGGCGCCATGCGCGTGCG | |
| | CGCTGGTCCTTTGGGCGCTAACTGCGTGCGCGCTGGGAAT | |
| | TGGCGCTAATTGCGCGTGCGCGCTGGGACTCAATGGCGCT | |
| | AATCGCGCGTGCGTTCTGGGGCCCGGGCGCTTGCGCCACT | |
| | TCCTGCCCGAGCCGCTGGCGCCCGAGGGTGTGGCCGCTGC | |
| | GTGCGCGCGCGCGACCCGGTCGCTGTTTGAACCGGGCGGA | |
| | GGCGGGGCTGGCGCCCGGTTGGGAGGGGGTTGGGGCCTGG | |
| | CTTCCTGCCGCGCGCCGCGGGGACGCCTCCGACCAGTGTT | |
| | TGCCTTTTATGGTAATAACGCGGCCGGCCCGGCTTCCTTT | |
| | GTCCCCAATCTGGGCGCGCGCCGGCGCCCCCTGGCGGCCT | |
| | AAGGACTCGGCGCGCCGGAAGTGGCCAGGGCGGCAGCGGC | |
| | TGCTCTTGGCGGCCCCGAGGTGACTATAGCCTTCTTTTGT | |
| | GTCTTGATAGTTCGCCAGCCTCTGCTAACCATGTTCATGC | |
| | CTTCTTCTTTTTCCTACAGCTCCTGGGCAACGTGCTGGTT | |
| | ATTGTGCTGTCTCATCATTTTGGCAAAGAATTC | |
| Chicken beta-actin | GTCGCTGCGCGCTGCCTTCGCCCCGTGCCCCGCTCCGCCG | 52 |
| exon/intron + rabbit | CCGCCTCGCGCCGCCCGCCCCGGCTCTGACTGACCGCGTT | |
| globin intron | ACTCCCACAGGTGAGCGGGCGGGACGGCCCTTCTCCTCCG | |
| | GGCTGTAATTAGCGCTTGGTTTAATGACGGCTTGTTTCTT | |
| | TTCTGTGGCTGCGTGAAAGCCTTGAGGGGCTCCGGGAGGG | |
| | CCCTTTGTGCGGGGGGAGCGGCTCGGGGGGTGCGTGCGTG | |
| | TGTGTGTGCGTGGGGAGCGCCGCGTGCGGCTCCGCGCTGC | |
| | CCGGCGGCTGTGAGCGCTGCGGGCGCGGCGCGGGGCTTTG | |
| | TGCGCTCCGCAGTGTGCGCGAGGGGAGCGCGGCCGGGGGC | |
| | GGTGCCCCGCGGTGCGGGGGGGGCTGCGAGGGGAACAAAG | |
| | GCTGCGTGCGGGGTGTGTGCGTGGGGGGGTGAGCAGGGGG | |
| | TGTGGGCGCGTCGGTCGGGCTGCAACCCCCCCTGCACCCC | |
| | CCTCCCCGAGTTGCTGAGCACGGCCCGGCTTCGGGTGCGG | |
| | GGCTCCGTACGGGGCGTGGCGCGGGGCTCGCCGTGCCGGG | |
| | CGGGGGGTGGCGGCAGGTGGGGGTGCCGGGCGGGGCGGGG | |
| | CCGCCTCGGGCCGGGGAGGGCTCGGGGGAGGGGCGCGGCG | |
| | GCCCCCGGAGCGCCGGCGGCTGTCGAGGCGCGGCGAGCCG | |
| | CAGCCATTGCCTTTTATGGTAATCGTGCGAGAGGGCGCAG | |
| | GGACTTCCTTTGTCCCAAATCTGTGCGGAGCCGAAATCTG | |
| | GGAGGCGCCGCCGCACCCCCTCTAGCGGGCGCGGGGCGAA | |
| | GCGGTGCGGCGCCGGCAGGAAGGAAATGGGCGGGGAGGGC | |
| | CTTCGTGCGTCGCCGCGCCGCCGTCCCCTTCTCCCTCTCC | |
| | AGCCTCGGGGCTGTCCGCGGGGGGACGGCTGCCTTCGGGG | |
| | GGGACGGGGCAGGGCGGGGTTCGGCTTCTGGCGTGTGACC | |
| | GGCGGCTCTAGAGCCTCTGCTAACCATGTTCATGCCTTCT | |
| | TCTTTTTCCTACAGCTCCTGGGCAACGTGCTGGTTATTGT | |
| | GCTGTCTCATCATTTTGGCAAAGAATTC | |
| SV40 intron | GGTAAGTTTAGTCTTTTTGTCTTTTATTTCAGGTCCCGGA | 53 |
| (Chimeric intron | TCCGGTGGTGGTGCAAATCAAAGAACTGCTCCTCAGTGGA | |
| sequence) | TGTTGCCTTTACTTCTAGGCCTGTACGGAAGTGTTACTTC | |
| Shown in FIG. 14 | TGCTCTAAAAGCTGCGGAATTGTACCCGC | |
| 5′UTR-Syn1 Hs | AGTCTGCGGTGGGCAGCGGAGGAGTCGTGTCGTGCCTGAG | 54 |
| | AGCGCAGCTGTGCTCCTGGGCACCGCGCAGTCCGCCCCCG | |
| | CGGCTCCTGGCCAGACCACCCCTAGGACCCCCTGCCCCAA | |
| | GTCGCA | |
| CMV IE exon | TCAGATCGCCTGGAGAGGCCATCCACGCTGTTTTGACCTC | 55 |
| | CATAGTGGACACCGGGACCGATCCAGCCTCCGCGGCCGGG | |
| | AACGGTGCATTGGAACGCGGATTCCCCGTGCCAAGAGTGA | |
| | C | |
| TPL-ePKP2 | CTCACTCTCTTCCGCATCGCTGTCTGCGAGGGCCAGCTGT | 56 |
| (adenovirus derived | TGGGCTCGCGGTTGAGGACAAACTCTTCGCGGTCTTTCCA | |
| enhancer element) | GTACTCTTGGATCGGAAACCCGTCGGCCTCCGAACGGTAC | |
| | TCCGCCACCGAGGGACCTGAGCGAGTCCGCATCGACCGGA | |
| | TCGGAAAACCTCTCGAGAAAGGCGTCTAACCAGTCACAGT | |
| | CGCAAGGTAGGCTGAGCACCGTGGCGGGCGGCAGCGGGTG | |
| | GCGGTCGGGGTTGTTTCTGGCGGAGGTGCTGCTGATGATG | |
| | TAATTAAAGTAGGCGGTCTTGAGACGGCGGATGGTCGAGG | |
| | TGAGGTGTGGCAGGCTTGAGATCCAGCTGTTGGGGTGAGT | |
| | ACTCCCTCTCAAAAGCGGGCATTACTTCTGCGCTAAGATT | |
| | GTCAGTTTCCAAAAACGAGGAGGATTTGATATTCACCTGG | |
| | CCCGATCTGGCCATACACTTGAGTGACAATGACATCCACT | |
| | TTGCCTTTCTCTCCACAGGTGTCCACTCCCAG | |
| Human EF1-α | CTTTTTCGCAACGGGTTTGCCGCCAGAACACAGGTAAGTG | 57 |
| intron/exon | CCGTGTGTGGTTCCCGCGGGCCTGGCCTCTTTACGGGTTA | |
| | TGGCCCTTGCGTGCCTTGAATTACTTCCACCTGGCTCCAG | |
| | TACGTGATTCTTGATCCCGAGCTGGAGCCAGGGGCGGGCC | |
| | TTGCGCTTTAGGAGCCCCTTCGCCTCGTGCTTGAGTTGAG | |
| | GCCTGGCCTGGGCGCTGGGGCCGCCGCGTGCGAATCTGGT | |
| | GGCACCTTCGCGCCTGTCTCGCTGCTTTCGATAAGTCTCT | |
| | AGCCATTTAAAATTTTTGATGACGTGCTGCGACGCTTTTT | |
| | TTCTGGCAAGATAGTCTTGTAAATGCGGGCCAGGATCTGC | |
| | ACACTGGTATTTCGGTTTTTGGGCCCGCGGCCGGCGACGG | |
| | GGCCCGTGCGTCCCAGCGCACATGTTCGGCGAGGCGGGGC | |
| | CTGCGAGCGCGGCCACCGAGAATCGGACGGGGGTAGTCTC | |
| | AAGCTGGCCGGCCTGCTCTGGTGCCTGGCCTCGCGCCGCC | |
| | GTGTATCGCCCCGCCCTGGGCGGCAAGGCTGGCCCGGTCG | |
| | GCACCAGTTGCGTGAGCGGAAAGATGGCCGCTTCCCGGCC | |
| | CTGCTCCAGGGGGCTCAAAATGGAGGACGCGGCGCTCGGG | |
| | AGAGCGGGCGGGTGAGTCACCCACACAAAGGAAAAGGGCC | |
| | TTTCCGTCCTCAGCCGTCGCTTCATGTGACTCCACGGAGT | |
| | ACCGGGCGCCGTCCAGGCACCTCGATTAGTTCTGGAGCTT | |
| | TTGGAGTACGTCGTCTTTAGGTTGGGGGGAGGGGTTTTAT | |
| | GCGATGGAGTTTCCCCACACTGAGTGGGTGGAGACTGAAG | |
| | TTAGGCCAGCTTGGCACTTGATGTAATTCTCCTTGGAATT | |
| | TGGCCTTTTTGAGTTTGGATCTTGGTTCATTCTCAAGCCT | |
| | CAGACAGTGGTTCAAAGTTTTTTTCTTCCATTTCAG | |
| HumanEF1-α, | GTAAGTGCCGTGTGTGGTTCCCGCGGGCCTGGCCTCTTTA | 58 |
| intron A | CGGGTTATGGCCCTTGCGTGCCTTGAATTACTTCCACCTG | |
| | GCTGCAGTACGTGATTCTTGATCCCGAGCTTCGGGTTGGA | |
| | AGTGGGTGGGAGAGTTCGAGGCCTTGCGCTTAAGGAGCCC | |
| | CTTCGCCTCGTGCTTGAGTTGAGGCCTGGCCTGGGCGCTG | |
| | GGGCCGCCGCGTGCGAATCTGGTGGCACCTTCGCGCCTGT | |
| | CTCGCTGCTTTCGATAAGTCTCTAGCCATTTAAAATTTTT | |
| | GATGACCTGCTGCGACGCTTTTTTTCTGGCAAGATAGTCT | |
| | TGTAAATGCGGGCCAAGATCTGCACACTGGTATTTCGGTT | |
| | TTTGGGGCCGCGGGCGGCGACGGGGCCCGTGCGTCCCAGC | |
| | GCACATGTTCGGCGAGGCGGGGCCTGCGAGCGCGGCCACC | |
| | GAGAATCGGACGGGGGTAGTCTCAAGCTGGCCGGCCTGCT | |
| | CTGGTGCCTGGCCTCGCGCCGCCGTGTATCGCCCCGCCCT | |
| | GGGCGGCAAGGCTGGCCCGGTCGGCACCAGTTGCGTGAGC | |
| | GGAAAGATGGCCGCTTCCCGGCCCTGCTGCAGGGAGCTCA | |
| | AAATGGAGGACGCGGCGCTCGGGAGAGCGGGCGGGTGAGT | |
| | CACCCACACAAAGGAAAAGGGCCTTTCCGTCCTCAGCCGT | |
| | CGCTTCATGTGACTCCACGGAGTACCGGGCGCCGTCCAGG | |
| | CACCTCGATTAGTTCTCGAGCTTTTGGAGTACGTCGTCTT | |
| | TAGGTTGGGGGGAGGGGTTTTATGCGATGGAGTTTCCCCA | |
| | CACTGAGTGGGTGGAGACTGAAGTTAGGCCAGCTTGGCAC | |
| | TTGATGTAATTCTCCTTGGAATTTGCCCTTTTTGAGTTTG | |
| | GATCTTGGTTCATTCTCAAGCCTCAGACAGTGGTTCAAAG | |
| | TTTTTTTCTTCCATTTCAG | |
| 5′UTR human | TCAGAAGCCCCGGGCTCGTCAGTCAAACCGGTTCTCTGTT | 59 |
| CamKIIa | TGCACTCGGCAGCACGGGCAGGCAAGTGGTCCCTAGGTTC | |
| | GGG | |
| B-globin intron | GTGAGTCTATGGGACCCTTGATGTTTTCTTTCCCCTTCTT | 60 |
| | TTCTATGGTTAAGTTCATGTCATAGGAAGGGGAGAAGTAA | |
| | CAGGGTACACATATTGACCAAATCAGGGTAATTTTGCATT | |
| | TGTAATTTTAAAAAATGCTTTCTTCTTTTAATATACTTTT | |
| | TTGTTTATCTTATTTCTAATACTTTCCCTAATCTCTTTCT | |
| | TTCAGGGCAATAATGATACAATGTATCATGCCTCTTTGCA | |
| | CCATTCTAAAGAATAACAGTGATAATTTCTGGGTTAAGGC | |
| | AATAGCAATATTTCTGCATATAAATATTTCTGCATATAAA | |
| | TTGTAACTGATGTAAGAGGTTTCATATTGCTAATAGCAGC | |
| | TACAATCCAGCTACCATTCTGCTTTTATTTTATGGTTGGG | |
| | ATAAGGCTGGATTATTCTGAGTCCAAGCTAGGCCCTTTTG | |
| | CTAATCATGTTCATACCTCTTATCTTCCTCCCACAG | |
| SV40 intron (long | TCTAGAGGATCCGGTACTCGAGGAACTGAAAAACCAGAAA | 61 |
| form; underlined 5′ | GTTAACTGGTAAGTTTAGTCTTTTTGTCTTTTATTTCAGG | |
| and 3′ extensions) | TCCCGGATCCGGTGGTGGTGCAAATCAAAGAACTGCTCCT | |
| | CAGTGGATGTTGCCTTTACTTCTAGGCCTGTACGGAAGTG | |
| | TTACTTCTGCTCTAAAAGCTGCGGAATTGTACCCGC | |

In some embodiments, the vector comprises a 3′ untranslated region selected from Table 5. In some embodiments, the vector genome comprises a polynucleotide sequence at least 75%, 80%, 85%, 90%, 92%, 93%, 94%, 95%, 96%, 97%, 98%, 99%, or 100% identical to any one of SEQ ID NOS 62-70.

TABLE 5

| | | SEQ |
|---|---|---|
| 3′ UNTRANSLATED | | ID |
| REGION | SEQUENCE | NO: |
| WPRE(x) (mutated | AATCAACCTCTGGATTACAAAATTTGTGAAAGATTGACTG | 62 |
| woodchuck hepatitis | GTATTCTTAACTATGTTGCTCCTTTTACGCTATGTGGATA | |
| regulatory element - | CGCTGCTTTAATGCCTTTGTATCATGCTATTGCTTCCCGT | |
| version 1) | ATGGCTTTCATTTTCTCCTCCTTGTATAAATCCTGGTTGC | |
| | TGTCTCTTTATGAGGAGTTGTGGCCCGTTGTCAGGCAACG | |
| | TGGCGTGGTGTGCACTGTGTTTGCTGACGCAACCCCCACT | |
| | GGTTGGGGCATTGCCACCACCTGTCAGCTCCTTTCCGGGA | |
| | CTTTCGCTTTCCCCCTCCCTATTGCCACGGCGGAACTCAT | |
| | CGCCGCCTGCCTTGCCCGCTGCTGGACAGGGGCTCGGCTG | |
| | TTGGGCACTGACAATTCCGTGGTGTTGTCGGGGAAATCAT | |
| | CGTCCTTTCCTTGGCTGCTCGCCTGTGTTGCCACCTGGAT | |
| | TCTGCGCGGGACGTCCTTCTGCTACGTCCCTTCGGCCCTC | |
| | AATCCAGCGGACCTTCCTTCCCGCGGCCTGCTGCCGGCTC | |
| | TGCGGCCTCTTCCGCGTCTTCGCCTTCGCCCTCAGACGAG | |
| | TCGGATCTCCCTTTGGGCCGCCTCCCCGC | |
| WPRE(x) (mutated | TCAACCTCTGGATTACAAAATTTGTGAAAGATTGACTGGT | 63 |
| woodchuck hepatitis | ATTCTTAACTATGTTGCTCCTTTTACGCTATGTGGATACG | |
| regulatory element - | CTGCTTTAATGCCTTTGTATCATGCTATTGCTTCCCGTAT | |
| version 2) | GGCTTTCATTTTCTCCTCCTTGTATAAATCCTGGTTGCTG | |
| | TCTCTTTATGAGGAGTTGTGGCCCGTTGTCAGGCAACGTG | |
| | GCGTGGTGTGCACTGTGTTTGCTGACGCAACCCCCACTGG | |
| | TTGGGGCATTGCCACCACCTGTCAGCTCCTTTCCGGGACT | |
| | TTCGCTTTCCCCCTCCCTATTGCCACGGCGGAACTCATCG | |
| | CCGCCTGCCTTGCCCGCTGCTGGACAGGGGCTCGGCTGTT | |
| | GGGCACTGACAATTCCGTGGTGTTGTCGGGGAAATCATCG | |
| | TCCTTTCCTTGGCTGCTCGCCTGTGTTGCCACCTGGATTC | |
| | TGCGCGGGACGTCCTTCTGCTACGTCCCTTCGGCCCTCAA | |
| | TCCAGCGGACCTTCCTTCCCGCGGCCTGCTGCCGGCTCTG | |
| | CGGCCTCTTCCGCGTCTTCGCCTTCGCCCTCAGACGAGTC | |
| | GGATCTCCCTTTGGGCCGCCTCCCCGCA | |
| WPRE(x) (mutated | TTCCTGTTAATCAACCTCTGGATTACAAAATTTGTGAAAG | 64 |
| woodchuck hepatitis | ATTGACTGGTATTCTTAACTATGTTGCTCCTTTTACGCTA | |
| regulatory element - | TGTGGATACGCTGCTTTAATGCCTTTGTATCATGCTATTG | |
| version 3) | CTTCCCGTATGGCTTTCATTTTCTCCTCCTTGTATAAATC | |
| | CTGGTTGCTGTCTCTTTATGAGGAGTTGTGGCCCGTTGTC | |
| | AGGCAACGTGGCGTGGTGTGCACTGTGTTTGCTGACGCAA | |
| | CCCCCACTGGTTGGGGCATTGCCACCACCTGTCAGCTCCT | |
| | TTCCGGGACTTTCGCTTTCCCCCTCCCTATTGCCACGGCG | |
| | GAACTCATCGCCGCCTGCCTTGCCCGCTGCTGGACAGGGG | |
| | CTCGGCTGTTGGGCACTGACAATTCCGTGGTGTTGTCGGG | |
| | GAAGCTGACGTCCTTTCCGCGGCTGCTCGCCTGTGTTGCC | |
| | ACCTGGATTCTGCGCGGGACGTCCTTCTGCTACGTCCCTT | |
| | CGGCCCTCAATCCAGCGGACCTTCCTTCCCGCGGCCTGCT | |
| | GCCGGCTCTGCGGCCTCTTCCGCCTCTTCGCCTTCGCCCT | |
| | CAGACGAGTCGGATCTCCCTTTGGGCCGCCTCCCCGCCCA | |
| | TGTATCTTTTTCACCTGTGCCTTGTTTTTGCCTGTGTTCC | |
| | GCGTCCTACTTTTCAAGCCTCCAAGCTGTGCCTTGGGCGG | |
| | CTTTGGGGCATGGACATAGATCCCTATAAAGAATTTGGTT | |
| | CATCTTATCAGTTGTTGAATTTTCTTCCTTTGGAC | |
| CAAX | TGTGTGATAATG | 65 |
| EES | CTGTTCTCATCACATCATATCAAGGTTATATACCATCAAT | 66 |
| | ATTGCCACAGATGTTACTTAGCCTTTTAATATTTCTCTAA | |
| | TTTAGTGTATATGCAATGATAGTTCTCTGATTTCTGAGAT | |
| | TGAGTTTCTCATGTGTAATGATTATTTAGAGTTTCTCTTT | |
| | CATCTGTTCAAATTTTTGTCTAGTTTTATTTTTTACTGAT | |
| | TTGTAAGACTTCTTTTTATAATCTGCATATTACAATTCTC | |
| | TTTACTGGGGTGTTGCAAATATTTTCTGTCATTCTATGGC | |
| | CTGACTTTTCTTAATGGTTTTTTAATTTTAAAAATAAGTC | |
| | TTAATATTCATGCAATCTAATTAACAATCTTTTCTTTGTG | |
| | GTTAGGACTTTGAGTCATAAGAAATTTTTCTCTACACTGA | |
| | AGTCATGATGGCATGCTTCTATATTATTTTCTAAAAGATT | |
| | TAAAGTTTTGCCTTCTCCATTTAGACTTATAATTCACTGG | |
| | AATTTTTTTGTGTGTATGGTATGACATATGGGTTCCCTTT | |
| | TATTTTTTACATATAAATATATTTCCCTGTTTTTCTAAAA | |
| | AAGAAAAAGATCATCATTTTCCCATTGTAAAATGCCATAT | |
| | TTTTTTCATAGGTCACTTACATATATCAATGGGTCTGTTT | |
| | CTGAGCTCTACTCTATTTTATCAGCCTCACTGTCTATCCC | |
| | CACACATCTCATGCTTTGCTCTAAATCTTGATATTTAGTG | |
| | GAACATTCTTTCCCATTTTGTTCTACAAGAATATTTTTGT | |
| | TATTGTCTTTGGGCTTTCTATATACATTTTGAAATGAGGT | |
| | TGACAAGTTA | |
| HPRE | ATAACAGGCCTATTGATTGGAAAGTTTGTCAACGAATTGT | 67 |
| | GGGTCTTTTGGGGTTTGCTGCCCCTTTTACGCAATGTGGA | |
| | TATCCTGCTTTAATGCCTTTATATGCATGTATACAAGCAA | |
| | AACAGGCTTTTACTTTCTCGCCAACTTACAAGGCCTTTCT | |
| | CAGTAAACAGTATATGACCCTTTACCCCGTTGCTCGGCAA | |
| | CGGCCTGGTCTGTGCCAAGTGTTTGCTGACGCAACCCCCA | |
| | CTGGTTGGGGCTTGGCCATAGGCCATCAGCGCATGCGTGG | |
| | AACCTTTGTGTCTCCTCTGCCGATCCATACTGCGGAACTC | |
| | CTAGCCGCTTGTTTTGCTCGCAGCAGGTCTGGAGCAAACC | |
| | TCATCGGGACCGACAATTCTGTCGTACTCTCCCGCAAGTA | |
| | TACATCGTTTCCATGGCTGCTAGGCTGTGCTGCCAACTGG | |
| | ATCCTGCGCGGGACGTCCTTTGTTTACGTCCCGTCGGCGC | |
| | TGAATCCCGCGGACGACCCCTCCCGGGGCCGCTTGGGGCT | |
| | CTACCGCCCGCTTCTCCGTCTGCCGTACCGTCCGACCACG | |
| | GGGCGCACCTCTCTTTACGCGGACTCCCCGTCTGTGCCTT | |
| | CTCATCTGCCGGACCGTGTGCACTTCGCTTCACCTCTGCA | |
| | CGTCGCATGGAGGCCACCGTGAACGCCCACCGGAACCTGC | |
| | CCAAGGTCTTGCATAAGAGGACTCTTGGACTTTCAGCAAT | |
| | GTCATC | |
| R2V17 (HepB derived | TTCCTGTAAACAGGCCTATTGATTGGAAAGTTTGTCAACG | 68 |
| enhancer element) | AATTGTGGGTCTTTTGGGGTTTGCTGCCCCTTTTACGCAA | |
| | TGTGGATATCCTGCTTTAATGCCTTTATATGCATGTATAC | |
| | AAGCAAAACAGGCTTTTACTTTCTCGCCAACTTACAAGGC | |
| | CTTTCTCAGTAAACAGTATATGACCCTTTACCCCGTTGCT | |
| | CGGCAACGGCCTGGTCTGTGCCAAGTGTTTGCTGACGCAA | |
| | CCCCCACTGGTTGGGGCTTGGCCATAGGCCATCAGCGCAT | |
| | GCGTGGAACCTTTGTGTCTCCTCTGCCGATCCATACTGCG | |
| | GAACTCCTAGCCGCTTGTTTTGCTCGCAGCTGGACTGGAG | |
| | CAAACCTCATCGGGACCGACAATTCTGTCGTACTCTCCCG | |
| | CAAGCACTCACCGTTTCCGCGGCTGCTCGCCTGTGTTGCC | |
| | ACCTGGATTCTGCGCGGGACGTCCTTCTGCTACGTCCCTT | |
| | CGGCCCTCAATCCAGCGGACCTTCCTTCCCGCGGCCTGCT | |
| | GCCGGCTCTGCGGCCTCTTCCGCCTCTTCGCCTTCGCCCT | |
| | CAGACGAGTCGGATCTCCCTTTGGGCCGCCTCCCCGCCCA | |
| | TGTATCTTTTTCACCTGTGCCTTGTTTTTGCCTGTGTTCC | |
| | GCGTCCTACTTTTCAAGCCTCCAAGCTGTGCCTTGGGCGG | |
| | CTTTGGGGCATGGACATAGATCCCTATAAAGAATTTGGTT | |
| | CATCTTATCAGTTGTTGAATTTTCTTCCTTTGGAC | |
| 3′UTR(globin) | GCTGGAGCCTCGGTAGCCGTTCCTCCTGCCCGCTGGGCCT | 69 |
| | CCCAACGGGCCCTCCTCCCCTCCTTGCACCGGCCCTTCCT | |
| | GGTCTTTGAATAAA | |
| WPRE(r) | ATTCGAGCATCTTACCGCCATTTATTCCCATATTTGTTCT | 70 |
| | GTTTTTCTTGATTTGGGTATACATTTAAATGTTAATAAAA | |
| | CAAAATGGTGGGGCAATCATTTACATTTTTAGGGATATGT | |
| | AATTACTAGTTCAGGTGTATTGCCACAAGACAAACATGTT | |
| | AAGAAACTTTCCCGTTATTTACGCTCTGTTCCTGTTAATC | |
| | AACCTCTGGATTACAAAATTTGTGAAAGATTGACTGATAT | |
| | TCTTAACTATGTTGCTCCTTTTACGCTGTGTGGATATGCT | |
| | GCTTTAATGCCTCTGTATCATGCTATTGCTTCCCGTACGG | |
| | CTTTCGTTTTCTCCTCCTTGTATAAATCCTGGTTGCTGTC | |
| | TCTTTATGAGGAGTTGTGGCCCGTTGTCCGTCAACGTGGC | |
| | GTGGTGTGCTCTGTGTTTGCTGACGCAACCCCCACTGGCT | |
| | GGGGCATTGCCACCACCTGTCAACTCCTTTCTGGGACTTT | |
| | CGCTTTCCCCCTCCCGATCGCCACGGCAGAACTCATCGCC | |
| | GCCTGCCTTGCCCGCTGCTGGACAGGGGCTAGGTTGCTGG | |
| | GCACTGATAATTCCGTGGTGTTGTCGGGGAAGGGCC | |

In some embodiments, the vector comprises a polyadenylation (polyA) signal selected from Table 6. In some embodiments, the polyA signal comprises a polynucleotide sequence at least 75%, 80%, 85%, 90%, 92%, 93%, 94%, 95%, 96%, 97%, 98%, 99%, or 100% identical to any one of SEQ ID NOS 71-75.

TABLE 6

| POLY- | | SEQ |
|---|---|---|
| ADENYLATION | | ID |
| SITE | SEQUENCE | NO: |
| Rabbit globin | TGGCTAATAAAGGAAATTTATTTTCATTGCAATAGTGTG | 71 |
| (pAGlobin-Oc) | TTGGAATTTTTTGTGTCTCTCACTCGGAAGAACATATGG | |
| | GAGGGCAAATCATTTAAAACATCAGAATGAGTATTTGGT | |
| | TTAGAGTTTGGCAACATATGCCCATATGCTGGCTGCCAT | |
| | GAACAAAGGTTGGCTATAAAGAGGTCATCAGTATATGAA | |
| | ACAGCCCCCTGCTGTCCATTCCTTATTCCATAGAAAAGC | |
| | CTTGACTTGAGGTTAGATTTTTTTTATATTTTGTTTTGT | |
| | GTTATTTTTTTCTTTAACATCCCTAAAATTTTCCTTACA | |
| | TGTTTTACTAGCCAGATTTTTCCTCCTCTCCTGACTACT | |
| | CCCAGTCATAGCTGTCCCTCTTCTCTTATGGAGATC | |
| Bovine growth | TTGCCAGCCATCTGTTGTTTGCCCCTCCCCCGTGCCTTC | 72 |
| hormone (pAGH-Bt - | CTTGACCCTGGAAGGTGCCACTCCCACTGTCCTTTCCTA | |
| version 1) | ATAAAATGAGGAAATTGCATCGCATTGTCTGAGTAGGTG | |
| | TCATTCTATTCTGGGGGGTGGGGTGGGGCAGGACAGCAA | |
| | GGGGGAGGATTGGGAATACAATAGCAGGCATGCTGGGGA | |
| | TGCGGTGGGCTCTATGGGTACCCAGGTGCTGAAGAATTG | |
| | ACCCGGTTCCTCCTGGG | |
| Bovine growth | TTGCCAGCCATCTGTTGTTTGCCCCTCCCCCGTGCCTTC | 73 |
| hormone (pAGH-Bt - | CTTGACCCTGGAAGGTGCCACTCCCACTGTCCTTTCCTA | |
| version 2) | ATAAAATGAGGAAATTGCATCGCATTGTCTGAGTAGGTG | |
| | TCATTCTATTCTGGGGGGTGGGGTGGGGCAGGACAGCAA | |
| | GGGGGAGGATTGGGAAGACAATAGCAGGCATGCTGGGGA | |
| | TGCGGTGGGCTCTATGGGTACCCAGGTGCTGAAGAATTG | |
| | ACCCGGTTCCTCCTGGG | |
| Bovine growth | CTGTGCCTTCTAGTTGCCAGCCATCTGTTGTTTGCCCCT | 74 |
| hormone (pAGH-Bt - | CCCCCGTGCCTTCCTTGACCCTGGAAGGTGCCACTCCCA | |
| version 3) | CTGTCCTTTCCTAATAAAATGAGGAAATTGCATCGCATT | |
| | GTCTGAGTAGGTGTCATTCTATTCTGGGGGGTGGGGTGG | |
| | GGCAGGACAGCAAGGGGGAGGATTGGGAAGACAATAGCA | |
| | GGCATGCTGGGGATGCGGTGGGCTCTATGG | |
| Human growth | CTGCCCGGGTGGCATCCCTGTGACCCCTCCCCAGTGCCT | 75 |
| hormone (pAGH-Hs) | CTCCTGGCCCTGGAAGTTGCCACTCCAGTGCCCACCAGC | |
| | CTTGTCCTAATAAAATTAAGTTGCATCATTTTGTCTGAC | |
| | TAGGTGTCCTTCTATAATATTATGGGGTGGAGGGGGGTG | |
| | GTATGGAGCAAGGGGCCCAAGTTGGGAAGAAACCTGTAG | |
| | GGCCTGC | |

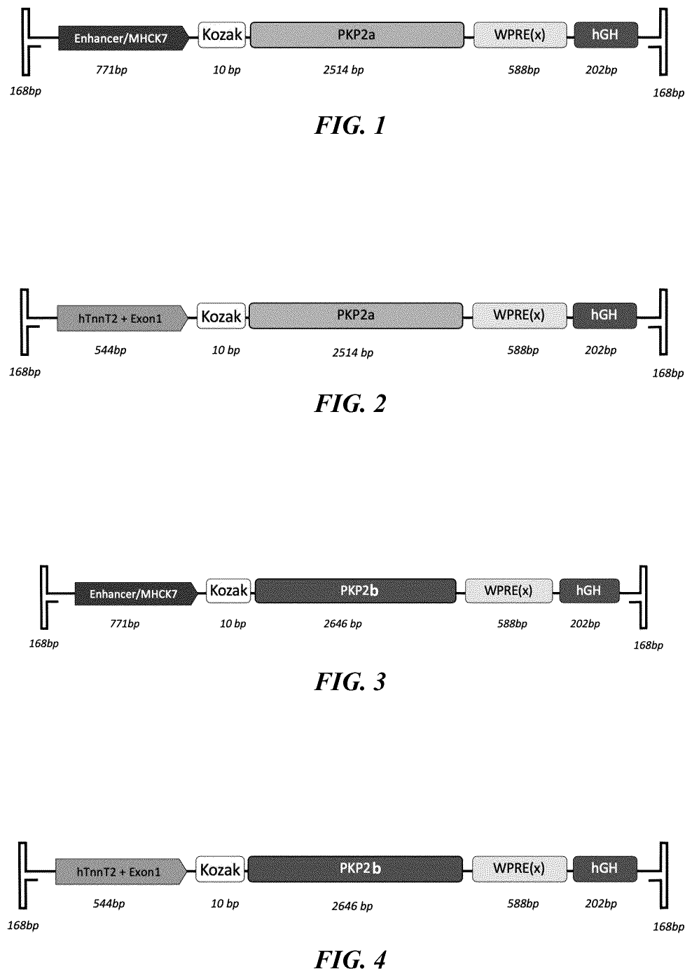

Illustrative vector genomes are depicted in FIGS. 1-4 and 11-14; and provided as SEQ ID NOs: 12-15 and 89-92. The expression cassette of each sequence is SEQ ID NOs: 8-11 or 93-96. In some embodiments, the vector genome comprises, consists essentially of, or consists of a polynucleotide sequence that shares at least 90%, 91%, 92%, 93%, 94%, 95%, 96%, 97%, 98%, 99%, or 100% identity to any one of SEQ ID NOs: 12-15 and 89-92, optionally with or without the ITR sequences. In some embodiments, the vector genome comprises, consists essentially of, or consists of a polynucleotide sequence that shares at least 90%, 91%, 92%, 93%, 94%, 95%, 96%, 97%, 98%, 99%, or 100% identity to any one of SEQ ID NOs: 8-11 and 93-96.

In a certain embodiment, the vector genome comprises, in 5′ to 3′ order, a 5′ITR; an MHCK7 promoter; a PKPa transgene; an WPRE(x) element; an pAGH-HS sequence; and a 3′ ITR. The vector genome may comprise, in 5′ to 3′ order, the polynucleotide sequences SEQ ID NO: 31; any one of SEQ ID NOs: 3, 6, and 87; SEQ ID NO: 63; and SEQ ID NO: 75; or polynucleotide sequences sharing 90%, 91%, 92%, 93%, 94%, 95%, 96%, 97%, 98%, 99%, or 100% identity to each of the foregoing. In certain embodiments, this vector genome is packaged in an AAV9 or AAVrh74 vector. The PKP2a transgene of this embodiment is a full length transgene, i.e. a transgene encoding a PKP of at least 800 or at least 830 amino acids.

In a certain embodiment, the vector genome comprises, in 5′ to 3′ order, a 5′ITR; a hTnnT2 promoter; a PKPa transgene; an WPRE(x) element; an pAGH-HS sequence; and a 3′ ITR. The vector genome may comprise, in 5′ to 3′ order, the polynucleotide sequences SEQ ID NO: 32 or 33; any one of SEQ ID NOs: 3, 6, and 87; SEQ ID NO: 63; and SEQ ID NO: 75; or polynucleotide sequences sharing 90%, 91%, 92%, 93%, 94%, 95%, 96%, 97%, 98%, 99%, or 100% identity to each of the foregoing. In certain embodiments, this vector genome is packaged in an AAV9 or AAVrh74 vector. The PKP2a transgene of this embodiment is a full length transgene, i.e. a transgene encoding a PKP of at least 800 or at least 830 amino acids.

In a certain embodiment, the vector genome comprises, in 5′ to 3′ order, a 5′ITR; an MHCK7 promoter; a PKPb transgene; an WPRE(x) element; an pAGH-HS sequence; and a 3′ ITR. The vector genome may comprise, in 5′ to 3′ order, the polynucleotide sequences SEQ ID NO: 31; any one of SEQ ID NOs: 4, 7, and 88; SEQ ID NO: 63; and SEQ ID NO: 75; or polynucleotide sequences sharing 90%, 91%, 92%, 93%, 94%, 95%, 96%, 97%, 98%, 99%, or 100% identity to each of the foregoing. In certain embodiments, this vector genome is packaged in an AAV9 or AAVrh74 vector. The PKP2b transgene of this embodiment is a full length transgene, i.e. a transgene encoding a PKP of at least 800 or at least 830 amino acids.

In a certain embodiment, the vector genome comprises, in 5′ to 3′ order, a 5′ ITR; a hTnnT2 promoter; a PKPb transgene; an WPRE(x) element; an pAGH-HS sequence; and a 3′ ITR. The vector genome may comprise, in 5′ to 3′ order, the polynucleotide sequences SEQ ID NO: 32 or 33; any one of SEQ ID NOs: 4, 7, and 88; SEQ ID NO: 63; and SEQ ID NO: 75; or polynucleotide sequences sharing 90%, 91%, 92%, 93%, 94%, 95%, 96%, 97%, 98%, 99%, or 100% identity to each of the foregoing. In certain embodiments, this vector genome is packaged in an AAV9 or AAVrh74 vector. The PKP2b transgene of this embodiment is a full length transgene, i.e. a transgene encoding a PKP of at least 800 or at least 830 amino acids.

In a certain embodiment, the vector genome comprises, in 5′ to 3′ order, a 5′ITR; an MHCK7 promoter; a PKPa transgene; optionally a WPRE element; a polyadenylation sequence; and a 3′ ITR. The vector genome may comprise, in 5′ to 3′ order, the polynucleotide sequences SEQ ID NO: 31; any one of SEQ ID NOs: 3, 6, and 87; or polynucleotide sequences sharing 90%, 91%, 92%, 93%, 94%, 95%, 96%, 97%, 98%, 99%, or 100% identity to each of the foregoing. In certain embodiments, this vector genome is packaged in an AAV9 or AAVrh74 vector. The PKP2a transgene of this embodiment is a full length transgene, i.e. a transgene encoding a PKP of at least 800 or at least 830 amino acids.

In a certain embodiment, the vector genome comprises, in 5′ to 3′ order, a 5′ITR; a hTnnT2 promoter; a PKPa transgene; optionally a WPRE element; a polyadenylation sequence; and a 3′ ITR. The vector genome may comprise, in 5′ to 3′ order, the polynucleotide sequences SEQ ID NO: 32 or 33; any one of SEQ ID NOs: 3, 6, and 87; or polynucleotide sequences sharing 90%, 91%, 92%, 93%, 94%, 95%, 96%, 97%, 98%, 99%, or 100% identity to each of the foregoing. In certain embodiments, this vector genome is packaged in an AAV9 or AAVrh74 vector. The PKP2a transgene of this embodiment is a full length transgene, i.e. a transgene encoding a PKP of at least 800 or at least 830 amino acids.

In a certain embodiment, the vector genome comprises, in 5′ to 3′ order, a 5′ITR; an MHCK7 promoter; a PKPb transgene; optionally a WPRE element; a polyadenylation sequence; and a 3′ ITR. The vector genome may comprise, in 5′ to 3′ order, the polynucleotide sequences SEQ ID NO: 31; any one of SEQ ID NOs: 4, 7, and 88; or polynucleotide sequences sharing 90%, 91%, 92%, 93%, 94%, 95%, 96%, 97%, 98%, 99%, or 100% identity to each of the foregoing. In certain embodiments, this vector genome is packaged in an AAV9 or AAVrh74 vector. The PKP2b transgene of this embodiment is a full length transgene, i.e. a transgene encoding a PKP of at least 800 or at least 830 amino acids.

In a certain embodiment, the vector genome comprises, in 5′ to 3′ order, a 5′ITR; a hTnnT2 promoter; a PKPb transgene; optionally a WPRE element; a polyadenylation sequence; and a 3′ ITR. The vector genome may comprise, in 5′ to 3′ order, the polynucleotide sequences SEQ ID NO: 32 or 33; any one of SEQ ID NOs: 4, 7, and 88; or polynucleotide sequences sharing 90%, 91%, 92%, 93%, 94%, 95%, 96%, 97%, 98%, 99%, or 100% identity to each of the foregoing. In certain embodiments, this vector genome is packaged in an AAV9 or AAVrh74 vector. The PKP2b transgene of this embodiment is a full length transgene, i.e. a transgene encoding a PKP of at least 800 or at least 830 amino acids.

In a certain embodiment, the vector genome comprises, in 5′ to 3′ order, a 5′ITR; an MHCK7 promoter; SV40 intron; a PKPa transgene; optionally a WPRE element; a polyadenylation sequence; and a 3′ ITR. The vector genome may comprise, in 5′ to 3′ order, the polynucleotide sequences SEQ ID NO: 31; SEQ ID NO: 53 or 61; any one of SEQ ID NOs: 3, 6, and 87; or polynucleotide sequences sharing 90%, 91%, 92%, 93%, 94%, 95%, 96%, 97%, 98%, 99%, or 100% identity to each of the foregoing. In certain embodiments, this vector genome is packaged in an AAV9 or AAVrh74 vector. The PKP2a transgene of this embodiment is a full length transgene, i.e. a transgene encoding a PKP of at least 800 or at least 830 amino acids.

In a certain embodiment, the vector genome comprises, in 5′ to 3′ order, a 5′ITR; a hTnnT2 promoter; SV40 intron; a PKPa transgene; optionally a WPRE element; a polyadenylation sequence; and a 3′ ITR. The vector genome may comprise, in 5′ to 3′ order, the polynucleotide sequences SEQ ID NO: 32 or 33; SEQ ID NO: 53 or 61; any one of SEQ ID NOs: 3, 6, and 87; or polynucleotide sequences sharing 90%, 91%, 92%, 93%, 94%, 95%, 96%, 97%, 98%, 99%, or 100% identity to each of the foregoing. In certain embodiments, this vector genome is packaged in an AAV9 or AAVrh74 vector. The PKP2a transgene of this embodiment is a full length transgene, i.e. a transgene encoding a PKP of at least 800 or at least 830 amino acids.

In a certain embodiment, the vector genome comprises, in 5′ to 3′ order, a 5′ ITR; an MHCK7 promoter; SV40 intron; a PKPb transgene; optionally a WPRE element; a polyadenylation sequence; and a 3′ ITR. The vector genome may comprise, in 5′ to 3′ order, the polynucleotide sequences SEQ ID NO: 31; SEQ ID NO: 53 or 61; any one of SEQ ID NOs: 4, 7, and 88; or polynucleotide sequences sharing 90%, 91%, 92%, 93%, 94%, 95%, 96%, 97%, 98%, 99%, or 100% identity to each of the foregoing. In certain embodiments, this vector genome is packaged in an AAV9 or AAVrh74 vector. The PKP2b transgene of this embodiment is a full length transgene, i.e. a transgene encoding a PKP of at least 800 or at least 830 amino acids.

In a certain embodiment, the vector genome comprises, in 5′ to 3′ order, a 5′ITR; a hTnnT2 promoter; SV40 intron; a PKPb transgene; optionally a WPRE element; a polyadenylation sequence; and a 3′ ITR. The vector genome may comprise, in 5′ to 3′ order, the polynucleotide sequences SEQ ID NO: 32 or 33; SEQ ID NO: 53 or 61; any one of SEQ ID NOs: 4, 7, and 88; or polynucleotide sequences sharing 90%, 91%, 92%, 93%, 94%, 95%, 96%, 97%, 98%, 99%, or 100% identity to each of the foregoing. In certain embodiments, this vector genome is packaged in an AAV9 or AAVrh74 vector. The PKP2b transgene of this embodiment is a full length transgene, i.e. a transgene encoding a PKP of at least 800 or at least 830 amino acids.

In each case the optionally WPRE element may be present or absent.

### Adeno-Associated Virus Vector

AAV vectors useful in the practice of the present invention can be packaged into AAV virions (viral particles) using various systems including adenovirus-based and helper-free systems. Standard methods in AAV biology include those described in Kwon and Schaffer. Pharm Res. (2008) 25(3):489-99; Wu et al. Mol. Ther. (2006) 14(3):316-27. Burger et al. Mol. Ther. (2004) 10(2):302-17; Grimm et al. Curr Gene Ther. (2003) 3(4):281-304; Deyle D R, Russell D W. Curr Opin Mol Ther. (2009) 11(4):442-447; McCarty et al. Gene Ther. (2001) 8(16):1248-54; and Duan et al. Mol Ther. (2001) 4(4):383-91. Helper-free systems included those described in U.S. Pat. Nos. 6,004,797; 7,588,772; and 7,094,604;

AAV DNA in the rAAV genomes may be from any AAV variant or serotype for which a recombinant virus can be derived including, but not limited to, AAV variants or serotypes AAV-1, AAV-2, AAV-3, AAV-4, AAV-5, AAV-6, AAV-7, AAV-8, AAV-9, AAV-10, AAV-11, AAV-12, AAV-13 and AAVrh10. Production of pseudotyped rAAV is disclosed in, for example, WO 01/83692. Other types of rAAV variants, for example rAAV with capsid mutations, are also contemplated. See, for example, Marsic et al., Molecular Therapy, 22(11):1900-1909 (2014). The nucleotide sequences of the genomes of various AAV serotypes are known in the art.

In some cases, the rAAV comprises a self-complementary genome. As defined herein, an rAAV comprising a “self-complementary” or “double stranded” genome refers to an rAAV which has been engineered such that the coding region of the rAAV is configured to form an intra-molecular double-stranded DNA template, as described in McCarty et al. Self-complementary recombinant adeno-associated virus (scAAV) vectors promoter efficient transduction independently of DNA synthesis. Gene Therapy. 8 (16): 1248-54 (2001). The present disclosure contemplates the use, in some cases, of an rAAV comprising a self-complementary genome because upon infection (such transduction), rather than waiting for cell mediated synthesis of the second strand of the rAAV genome, the two complementary halves of scAAV will associate to form one double stranded DNA (dsDNA) unit that is ready for immediate replication and transcription. It will be understood that instead of the full coding capacity found in rAAV (4.7-6 kb), rAAV comprising a self-complementary genome can only hold about half of that amount (≈2.4 kb).

In other cases, the rAAV vector comprises a single stranded genome. As defined herein, a “single standard” genome refers to a genome that is not self-complementary. In most cases, non-recombinant AAVs have singled stranded DNA genomes. There have been some indications that rAAVs should be scAAVs to achieve efficient transduction of cells. The present disclosure contemplates, however, rAAV vectors that maybe have singled stranded genomes, rather than self-complementary genomes, with the understanding that other genetic modifications of the rAAV vector may be beneficial to obtain optimal gene transcription in target cells. In some cases, the present disclosure relates to single-stranded rAAV vectors capable of achieving efficient gene transfer to anterior segment in the mouse eye. See Wang et al. Single stranded adeno-associated virus achieves efficient gene transfer to anterior segment in the mouse eye. PLOS ONE 12(8): e0182473 (2017).

In some cases, the rAAV vector is of the serotype AAV1, AAV2, AAV4, AAV5,AAV6, AAV7, AAV8, AAV9, AAV10, AAV11, AAV12, AAV13, AAVrh10, or AAVrh74. Production of pseudotyped rAAV is disclosed in, for example, WO 01/83692. Other types of rAAV variants, for example rAAV with capsid mutations, are also contemplated. See, for example, Marsic et al., Molecular Therapy, 22(11): 1900-1909 (2014). In some cases, the rAAV vector is of the serotype AAV9. In some embodiments, said rAAV vector is of serotype AAV9 and comprises a single stranded genome. In some embodiments, said rAAV vector is of serotype AAV9 and comprises a self-complementary genome. In some embodiments, a rAAV vector comprises the inverted terminal repeat (ITR) sequences of AAV2. In some embodiments, the rAAV vector comprises an AAV2 genome, such that the rAAV vector is an AAV-2/9 vector, an AAV-2/6 vector, or an AAV-2/8 vector.

Full-length sequences and sequences for capsid genes for most known AAVs are provided in U.S. Pat. No. 8,524,446, which is incorporated herein in its entirety.

AAV vectors may comprise wild-type AAV sequence or they may comprise one or more modifications to a wild-type AAV sequence. In certain embodiments, an AAV vector comprises one or more amino acid modifications, optionally substitutions, deletions, or insertions, within a capsid protein, optionally VP1, VP2 and/or VP3. In particular embodiments, the modification provides for reduced immunogenicity when the AAV vector is provided to a subject.

Capsid proteins of a rAAV may be modified so that the rAAV is targeted to a particular target tissue of interest such as endothelial cells or more particularly endothelial tip cells. In some embodiments, the rAAV is directly injected into the intracerebroventricular space of the subject.

In some embodiments, the rAAV virion is an AAV2 rAAV virion. The capsid many be an AAV2 capsid or functional variant thereof. In some embodiments, the AAV2 capsid shares at least 98%, 99%, or 100% identity to a reference AAV2 capsid, e.g., SEQ ID NO: 76.

In some embodiments, the rAAV virion is an AAV9 rAAV virion. The capsid many be an AAV9 capsid or functional variant thereof. In some embodiments, the AAV9 capsid shares at least 98%, 99%, or 100% identity to a reference AAV9 capsid, e.g., SEQ ID NO: 77.

In some embodiments, the rAAV virion is an AAV6 rAAV virion. The capsid many be an AAV9 capsid or functional variant thereof. In some embodiments, the AAV6 capsid shares at least 98%, 99%, or 100% identity to a reference AAV6 capsid, e.g., SEQ ID NO: 78.

In some embodiments, the rAAV virion is an AAVrh. 10 rAAV virion. The capsid many be an AAV9 capsid or functional variant thereof. In some embodiments, the AAVrh. 10capsid shares at least 98%, 99%, or 100% identity to a reference AAVrh. 10 capsid, e.g., SEQ ID NO: 79.

In some embodiments, the capsid protein is encoded by a polynucleotide supplied on a plasmid in trans to the transfer plasmid. The polynucleotide sequence of wild-type AAVrh74 cap is provided as SEQ ID NO: 80.

The disclosure further provides protein sequences for AAVrh74 VP1, VP2, and VP3, including SEQ ID NOs: 81-83, and homologs or functional variants thereof.

In certain cases, the AAVrh74 capsid comprises the amino acid sequence set forth in SEQ ID NO: 81. In some embodiments, the rAAV vector comprises a polypeptide that comprises, or consists essentially of, or yet further consists of a sequence, e.g., at least 65%, at least 70%, at least 75%, at least 80%, 81%, 82%, 83%, 84%, 85%, 86%, 87%, 88%, or 89%, more typically 90%, 91%, 92%, 93%, 94%, 95%, 96%, 97%, 98%, 99% or more identical to amino acid sequence of AAVrh74 VP1 which is set forth in SEQ ID NO: 81. In some embodiments, the rAAV vector comprises a polypeptide that comprises, or consists essentially of, or yet further consists of a sequence, e.g., at least 65%, at least 70%, at least 75%, at least 80%, 81%, 82%, 83%, 84%, 85%, 86%, 87%, 88%, or 89%, more typically 90%, 91%, 92%, 93%, 94%, 95%, 96%, 97%, 98%, 99% or more identical to amino acid sequence of AAVrh74 VP2 which is set forth in SEQ ID NO: 82. In some embodiments, the rAAV vector comprises a polypeptide that comprises, or consists essentially of, or yet further consists of a sequence, e.g., at least 65%, at least 70%, at least 75%, at least 80%, 81%, 82%, 83%, 84%, 85%, 86%, 87%, 88%, or 89%, more typically 90%, 91%, 92%, 93%, 94%, 95%, 96%, 97%, 98%, 99% or more identical to amino acid sequence of AAVrh74 VP3 which is set forth in SEQ ID NO: 83.

In some embodiments, the rAAV virion is an AAV-PHP.B rAAV virion or a neutrotrophic variant thereof, such as, without limitation, those disclosed in Int'l Pat. Pub. Nos. WO 2015/038958 A1 and WO 2017/100671 A1. For example, the AAV capsid may comprise at least 4 contiguous amino acids from the sequence TLAVPFK (SEQ ID NO:85) or KFPVALT (SEQ ID NO:86), e.g., inserted between a sequence encoding for amino acids 588 and 589 of AAV9.

The capsid many be an AAV-PHP.B capsid or functional variant thereof. In some embodiments, the AAV-PHP.B capsid shares at least 98%, 99%, or 100% identity to a reference AAV-PHP.B capsid, e.g., SEQ ID NO: 84.

Further AAV capsids used in the rAAV virions of the disclosure include those disclosed in Pat. Pub. Nos. WO 2009/012176 A2 and WO 2015/168666 A2.

Without being bound by theory, the present inventors have determined that an AAV9 vector, AAVrh.74, or an AAVrh. 10 vector will confer desirable cardiac tropism on the vector. Without being bound by theory, the present inventors have further determined that an AAV9 vector, AAVrh.74, or an AAVrh. 10 vector may provide desired specificity to cardiac cells.

In an aspect, the disclosure provides pharmaceutical compositions comprising the rAAV virion of the disclosure and one or more pharmaceutically acceptable carriers, diluents, or excipients.

For purposes of administration, optionally by injection, various solutions can be employed, such as sterile aqueous solutions. Such aqueous solutions can be buffered, if desired, and the liquid diluent first rendered isotonic with saline or glucose. Solutions of rAAV as a free acid (DNA contains acidic phosphate groups) or a pharmacologically acceptable salt can be prepared in water suitably mixed with a surfactant such as Poloxamer 188, e.g., at 0.001% or 0.01%. A dispersion of rAAV can also be prepared in glycerol, liquid polyethylene glycols and mixtures thereof and in oils. Under ordinary conditions of storage and use, these preparations contain a preservative to prevent the growth of microorganisms. In this connection, the sterile aqueous media employed are all readily obtainable by standard techniques well-known to those skilled in the art.

The pharmaceutical forms suitable for injectable use include but are not limited to sterile aqueous solutions or dispersions and sterile powders for the extemporaneous preparation of sterile injectable solutions or dispersions. In all cases the form is sterile and must be fluid to the extent that easy syringability exists. It must be stable under the conditions of manufacture and storage and must be preserved against the contaminating actions of microorganisms such as bacteria and fungi. The carrier can be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride. Prolonged absorption of the injectable compositions can be brought about by use of agents delaying absorption, for example, aluminum monostearate and gelatin.

Sterile injectable solutions may be prepared by incorporating rAAV in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the certain methods of preparation are vacuum drying and the freeze-drying technique that yield a powder of the active ingredient plus any additional desired ingredient from the previously sterile-filtered solution thereof.

In another aspect, the disclosure comprises a kit comprising an rAAV virion of the disclosure and instructions for use.

In an aspect, the disclosure provides a method of increasing PKP2 activity in a cell, comprising contacting the cell with an rAAV of the disclosure. In another aspect, the disclosure provides a method of increasing PKP2 activity in a subject, comprising administering to the subject an rAAV of the disclosure. In some embodiments, the cell and/or subject is deficient in PKP2 messenger RNA or PKP2 protein expression levels and/or activity and/or comprises a loss-of-function mutation in PKP2. The cell may be a cardiac cell, e.g. a cardiomyocyte cell. In particular embodiments, the subject is a mammal, e.g., a human.

In some embodiments, the method promotes survival of cardiac cell, e.g. a cardiomyocyte cell, in cell culture and/or in vivo. In some embodiments, the method promotes and/or restores function of the heart.

In another aspect, the disclosure provides a method of treating a disease or disorder in a subject in need thereof, comprising administering to the subject an effective amount of an rAAV virion of the disclosure. In some embodiments, the disease or disorder is a cardiac disease or disorder. Illustrative cardiac disorders include heart failure, arrhythmogenic right ventricular cardiomyopathy (ARVC), Brugada syndrome (BrS) and idiopathic ventricular fibrillation. In certain embodiments, the subject suffers from or is at risk for arrhythmogenic right ventricular cardiomyopathy (ARVC). In particular embodiments, the subject is a mammal, e.g., a human, having a loss-of-function mutation in a PKP2 gene. In particular methods, treatment with the rAAV virion results in expression of the PKP2 protein encoded by the rAAV virion in the subject, e.g., in the subject's heart or cardiac tissue. In certain embodiments, treatment with the rAAV virion results in at least two-fold, at least five-fold, at least ten-fold, or more PKP2 protein levels detectable in the subject's heart.

The AAV-mediated delivery of PKP2 protein to the heart may increase life span, prevent or attenuate cardiac cell degeneration, heart failure, scarring, reduced ejection fraction, arrythmia, angina, exercise intolerance, angina (chest pain), sudden cardiac death, exertional myalgias and cramps. The AAV-mediated delivery of PKP2 protein to the heart may show improvement from, or prevent normal disease course detected by use of pathological electrocardiogram, cardiac MRI, heart biopsy, decrease in paroxysmal ventricular arrhythmias, decrease in sudden cardiac death, and/or decrease in or lack of further development of fibro-fatty deposits in right ventricular myocardium. The methods of the disclosure may prevent a decrease in, restore, and/or increase right ventricular ejection fraction (RVEF).

The methods disclosed herein may provide efficient biodistribution in the heart. They may result in sustained in expression in all, or a substantial fraction of, cardiac cells, e.g., cardiomyocytes. Notably, the methods disclosed herein may provide long-lasting expression of PKP2 protein throughout the life of the subject following AAV vector administration. In some embodiments, PKP2 protein expression in response to treatment lasts at least 1, 2, 3, 4, 5, 6, 7, 8, 9, 10, 15, 20, 25, 30, 35, or 40 years.

Combination therapies are also contemplated by the invention. Combinations of methods of the invention with standard medical treatments (e.g., corticosteroids or topical pressure reducing medications) are specifically contemplated, as are combinations with novel therapies. In some cases, a subject may be treated with a steroid and/or combination of immune suppressing agents to prevent or to reduce an immune response to administration of a rAAV described herein.

In some embodiments, the AAV vector is administered at a dose of between about 1×10¹² and 5×10¹⁴ vector genomes (vg) or between about 1×10¹² and 6×10¹⁴ vg of the AAV vector per kilogram (vg) of total body mass of the subject (vg/kg). In some embodiments, the AAV vector is administered at a dose of between about 1×10¹³ and 5×10¹⁴ vg/kg. In some embodiments, the AAV vector is administered at a dose of between about 5×10¹³ and 3×10¹⁴ vg/kg. In some embodiments, the AAV vector is administered at a dose of between about 5×10¹³ and 1×10¹⁴ vg/kg. In some embodiments, the AAV vector is administered at a dose of less than about 1×10¹² vg/kg, less than about 3×10¹² vg/kg, less than about 5×10¹² vg/kg, less than about 7×10¹² vg/kg, less than about 1×10¹³ vg/kg, less than about 3×10¹³ vg/kg, less than about 5×10¹³ vg/kg, less than about 7×10¹³ vg/kg, less than about 1×10¹⁴ vg/kg, less than about 3×10¹⁴ vg/kg, less than about 5×10¹⁴ vg/kg, less than about 7×10¹⁴ vg/kg, less than about 1×10¹⁵ vg/kg, less than about 3×10¹⁵ vg/kg, less than about 5×10¹⁵ vg/kg, or less than about 7×10¹⁵ vg/kg. In certain embodiments, the AAV vector delivered at any of these doses is an AAV9 vector or an AAV rh74 vector. In some cases, it may be advantageous to use a higher dose for an AAV rh74 vector than for an AAV9 vector.

In some embodiments, the AAV vector is administered at a dose of about 1×10¹² vg/kg, about 3×10¹² vg/kg, about 5×10¹² vg/kg, about 7×10¹² vg/kg, about 1×10¹³ vg/kg, about 3×10¹³ vg/kg, about 5×10¹³ vg/kg, about 7×10¹³ vg/kg, about 1×10¹⁴ vg/kg, about 3×10¹⁴ vg/kg, about 5×10¹⁴ vg/kg, about 7×10¹⁴ vg/kg, about 1×10¹⁵ vg/kg, about 3×10¹⁵ vg/kg, about 5×10¹⁵ vg/kg, or about 7×10¹⁵ vg/kg. In certain embodiments, the AAV vector delivered at any of these doses is an AAV9 vector or an AAV rh74 vector.

In some embodiments, the AAV vector is administered at a dose of 1×10¹² vg/kg, 3×10¹² vg/kg, 5×10¹² vg/kg, 7×10¹² vg/kg, 1×10¹³ vg/kg, 3×10¹³ vg/kg, 5×10¹³ vg/kg, 7×10¹³ vg/kg, 1×10¹⁴ vg/kg, 3×10¹⁴ vg/kg, 5×10¹⁴ vg/kg, 7×10¹⁴ vg/kg, 1×10¹⁵ vg/kg, 3×10¹⁵ vg/kg, 5×10¹⁵ vg/kg, or 7×10¹⁵ vg/kg. In certain embodiments, the AAV vector delivered at any of these doses is an AAV9 vector or an AAV rh74 vector.

In some embodiments, the AAV vector is administered systemically at a dose of between about 1×10¹² and 5×10¹⁴ vector genomes (vg) of the AAV vector per kilogram (vg) of total body mass of the subject (vg/kg). In some embodiments, the AAV vector is administered systemically at a dose of between about 1×10¹³ and 5×10¹⁴ vg/kg. In some embodiments, the AAV vector is administered systemically at a dose of between about 5×10¹³ and 3×10¹⁴ vg/kg. In some embodiments, the AAV vector is administered systemically at a dose of between about 5×10¹³ and 1×10¹⁴ vg/kg. In some embodiments, the AAV vector is administered systemically at a dose of less than about 1×10¹² vg/kg, less than about 3×10¹² vg/kg, less than about 5×10¹² vg/kg, less than about 7×10¹² vg/kg, less than about 1×10¹³ vg/kg, less than about 3×10¹³ vg/kg, less than about 5×10¹³ vg/kg, less than about 7×10¹³ vg/kg, less than about 1×10¹⁴ vg/kg, less than about 3×10¹⁴ vg/kg, less than about 5×10¹⁴ vg/kg, less than about 7×10¹⁴ vg/kg, less than about 1×10¹⁵ vg/kg, less than about 3×10¹⁵ vg/kg, less than about 5×10¹⁵ vg/kg, or less than about 7×10¹⁵ vg/kg. In certain embodiments, the AAV vector delivered at any of these doses is an AAV9 vector or an AAV rh74 vector.

In some embodiments, the AAV vector is administered systemically at a dose of about 1×10¹² vg/kg, about 3×10¹² vg/kg, about 5×10¹² vg/kg, about 7×10¹² vg/kg, about 1×10¹³ vg/kg, about 3×10¹³ vg/kg, about 5×10¹³ vg/kg, about 7×10¹³ vg/kg, about 1×10¹⁴ vg/kg, about 3×10¹⁴ vg/kg, about 5×10¹⁴ vg/kg, about 7×10¹⁴ vg/kg, about 1×10¹⁵ vg/kg, about 3×10¹⁵ vg/kg, about 5×10¹⁵ vg/kg, or about 7×10¹⁵ vg/kg. In certain embodiments, the AAV vector delivered at any of these doses is an AAV9 vector or an AAV rh74 vector.

In some embodiments, the AAV vector is administered systemically at a dose of 1×10¹² vg/kg, 3×10¹² vg/kg, 5×10¹² vg/kg, 7×10¹² vg/kg, 1×10¹³ vg/kg, 3×10¹³ vg/kg, 5×10¹³ vg/kg, 7×10¹³ vg/kg, 1×10¹⁴ vg/kg, 3×10¹⁴ vg/kg, 5×10¹⁴ vg/kg, 7×10¹⁴ vg/kg, 1×10¹⁵ vg/kg, 3×10¹⁵ vg/kg, 5×10¹⁵ vg/kg, or 7×10¹⁵ vg/kg. In certain embodiments, the AAV vector delivered at any of these doses is an AAV9 vector or an AAV rh74 vector.

In some embodiments, the AAV vector is administered intravenously at a dose of between about 1×10¹² and 5×10¹⁴ vector genomes (vg) of the AAV vector per kilogram (vg) of total body mass of the subject (vg/kg). In some embodiments, the AAV vector is administered intravenously at a dose of between about 1×10¹³ and 5×10¹⁴ vg/kg. In some embodiments, the AAV vector is administered intravenously at a dose of between about 5×10¹³ and 3×10¹⁴ vg/kg. In some embodiments, the AAV vector is administered intravenously at a dose of between about 5×10¹³ and 1×10¹⁴ vg/kg. In some embodiments, the AAV vector is administered intravenously at a dose of less than about 1×10¹² vg/kg, less than about 3×10¹² vg/kg, less than about 5×10¹² vg/kg, less than about 7×10¹² vg/kg, less than about 1×10¹³ vg/kg, less than about 3×10¹³ vg/kg, less than about 5×10¹³ vg/kg, less than about 7×10¹³ vg/kg, less than about 1×10¹⁴ vg/kg, less than about 3×10¹⁴ vg/kg, less than about 5×10¹⁴ vg/kg, less than about 7×10¹⁴ vg/kg, less than about 1×10¹⁵ vg/kg, less than about 3×10¹⁵ vg/kg, less than about 5×10¹⁵ vg/kg, or less than about 7×10¹⁵ vg/kg. In certain embodiments, the AAV vector delivered at any of these doses is an AAV9 vector or an AAV rh74 vector.

In some embodiments, the AAV vector is administered intravenously at a dose of about 1×10¹² vg/kg, about 3×10¹² vg/kg, about 5×10¹² vg/kg, about 7×10¹² vg/kg, about 1×10¹³ vg/kg, about 3×10¹³ vg/kg, about 5×10¹³ vg/kg, about 7×10¹³ vg/kg, about 1×10¹⁴ vg/kg, about 3×10¹⁴ vg/kg, about 5×10¹⁴ vg/kg, about 7×10¹⁴ vg/kg, about 1×10¹⁵ vg/kg, about 3×10¹⁵ vg/kg, about 5×10¹⁵ vg/kg, or about 7×10¹⁵ vg/kg.

In some embodiments, the AAV vector is administered intravenously at a dose of 1×10¹² vg/kg, 3×10¹² vg/kg, 5×10¹² vg/kg, 7×10¹² vg/kg, 1×10¹³ vg/kg, 3×10¹³ vg/kg, 5×10¹³ vg/kg, 7×10¹³ vg/kg, 1×10¹⁴ vg/kg, 3×10¹⁴ vg/kg, 5×10¹⁴ vg/kg, 7×10¹⁴ vg/kg, 1×10¹⁵ vg/kg, 3×10¹⁵ vg/kg, 5×10¹⁵ vg/kg, or 7×10¹⁵ vg/kg. In certain embodiments, the AAV vector delivered at any of these doses is an AAV9 vector or an AAV rh74 vector.

Evidence of functional improvement, clinical benefit or efficacy in patients may be revealed by change in New York Heart Association functional classification (NYHA Class), pathological electrocardiogram, cardiac MRI, heart biopsy, decrease in paroxysmal ventricular arrhythmias, decrease in sudden cardiac death, and/or decrease in or lack of further development of fibro-fatty deposits in right ventricular myocardium. Benefit may be observed in electrocardiogramormally associated with arrhythmogenic right ventricular cardiomyopathy such as T wave inversion, prolonged S-wave upstroke, localized QRS widening, and/or paroxysmal episodes of ventricular tachycardia.

In some embodiments, the method prevents or reduces a decrease in left ventricle ejection fraction percentage (LVEF %), optionally by about 50%, about 60%, about 70%, about 80%, about 90%, or about 100% compared to the decrease observed in an untreated subject suffering from or at risk for disease or disorder related to or caused by loss of function in PKP2.

In some embodiments, the method prevents or reduces a decrease in left ventricle fractional shortening percentage (FS %), optionally by about 50%, about 60%, about 70%, about 80%, about 90%, or about 100% compared to the decrease observed in an untreated subject suffering from or at risk for disease or disorder related to or caused by loss of function in PKP2.

In some embodiments, the method prevents or reduces an increase in right ventricle area in millimeters squared (RV Area (mm2), optionally by about 50%, about 60%, about 70%, about 80%, about 90%, or about 100% compared to the increase observed in an untreated subject suffering from or at risk for disease or disorder related to or caused by loss of function in PKP2.

In some embodiments, the method prevents or reduces a decrease in right ventricle velocity time integral in millimeters per second (RV VTI (mm/sec), optionally by about 50%, about 60%, about 70%, about 80%, about 90%, or about 100% compared to the decrease observed in an untreated subject suffering from or at risk for disease or disorder related to or caused by loss of function in PKP2.

In some embodiments, the method prevents or reduces an increase in left ventricle or right ventricle fibrosis, optionally by about 50%, about 60%, about 70%, about 80%, about 90%, or about 100% compared to the increase observed in an untreated subject suffering from or at risk for disease or disorder related to or caused by loss of function in PKP2.

Administration of an effective dose of the compositions may be by routes standard in the art including, but not limited to, systemic, local, direct injection, intravenous, intracardiac administration. In some cases, administration comprises systemic, local, direct injection, intravenous, intracardiac injection. Administration may be performed by cardiac catheterization.

In some embodiments, the disclosure provides for local administration and systemic administration of an effective dose of rAAV and compositions of the invention. For example, systemic administration may be administration into the circulatory system so that the entire body is affected. Systemic administration includes parental administration through injection, infusion or implantation. Routes of administration for the compositions disclosed herein include intravenous (“IV”) administration, intraperitoneal (“IP”) administration, intramuscular (“IM”) administration, intralesional administration, or subcutaneous (“SC”) administration, or the implantation of a slow-release device, e.g., a mini-osmotic pump, a depot formulation, etc. In some embodiments, the methods of the disclosure comprise administering an AAV vector of the disclosure, or pharmaceutical composition thereof by intravenous, intramuscular, intraarterial, intrarenal, intraurethral, intracardiac, intracoronary, intramyocardial, intradermal, epidural, subcutaneous, intraperitoneal, intraventricular, ionophoretic or intracranial administration.

In particular, administration of rAAV of the present invention may be accomplished by using any physical method that will transport the rAAV recombinant vector into the target tissue of an animal. Administration includes, but is not limited to, injection into the heart.

In some embodiments, the methods of the disclosure comprise intracardiac delivery. Infusion may be performed using specialized cannula, catheter, syringe/needle using an infusion pump. Administration may comprise delivery of an effective amount of the rAAV virion, or a pharmaceutical composition comprising the rAAV virion, to the heart. These may be achieved, e.g., via intravenous, intramuscular, intraarterial, intrarenal, intraurethral, intracardiac, intracoronary, intramyocardial, intradermal, epidural, subcutaneous, intraperitoneal, intraventricular, ionophoretic or intracranial administration. The compositions of the disclosure may further be administered intravenously.

The method of treatment disclosed herein may reduce and/or prevent one or more symptoms including but not limited to ventricular hypertrophy, ventricular tachycardia, exercise intolerance, angina, and reduced RVEF.

### EXAMPLES

### Example 1: Pre-Clinical Bioactivity and Efficacy

Vectors illustrated in FIGS. 1-4 are tested. AAV vectors or respective expression cassettes are tested in vitro using cultured cardiomyocytes (e.g., induced pluripotent stem cell cardiomyocytes, iPSC-CMs) or other cells amenable to transfection or transduction with these constructs. Expression of PKP2 is assessed by immunofluorescence and Western blot. Cell-based studies employing patient iPSC-derived cardiomyocytes will reveal benefit of overexpression of PKP2 transgene (either following AAV vector transduction and/or transfection with vector plasmids) by a decreased adipogenic potential (e.g. less lipid accumulation), decreased upregulation or abnormal peroxisome proliferator-activated receptor gamma activation, associated with increased density of PKP2.

Selected vectors are tested in vivo using mutant mouse models of cardiomyopathy (e.g., PKP2-cKO, among others). Evidence of benefit of AAV mediated overexpression of PKP2 may be revealed using a cardiomyocyte-specific, tamoxifen-activated, PKP2 knockout murine line, referred to as “PKP2-cKO”. This mouse model allows control of the onset of PKP2 loss of expression, limits loss of PKP2 to adult myocytes, and initiates a progression of molecular and functional events leading to an arrhythmogenic cardiomyopathy, with right ventricular predominance in this mouse. Additional mouse models that result in similar course of pathology may also be utilized to reveal benefit of AAV-mediated overexpression of PKP2 in cardiomyocytes. Benefit of AAV-mediated PKP2 overexpression would be evidenced by increase in survival, mitigation of the normal progression of cardiomyopathy observed on echocardiograms from left and/or right ventricle (e.g. greater left ventricular ejection fraction, greater left ventricle fractional shortening, and greater right ventricle velocity time interval, compared to PKP cKO formulation buffer control animals).

Electrophysiological evidence of functional benefit of AAV-mediated delivery of PKP2 protein is demonstrated by mitigation of disease-related disrupted calcium dynamics in affected cardiomyocytes, most notably on measures of L-type calcium current, sarcoplasmic reticulum calcium leak, diastolic calcium leak, as well as standard measures of calcium transients in affected (e.g., PKP2-deficient) cardiomyocytes such as time to peak amplitude and relaxation time constants. Histological analyses will reveal benefit of AAV-mediated PKP2 overexpression by diminished appearance of disease-related collagen deposition (e.g., via trichrome stain) in various regions of the heart including ventricles, compared to cKO formulation buffer injected controls. Additional benefit will also be revealed by evaluating cardiomyocyte ventricular proteins involved in calcium signaling pathways, measured by increased (i.e., normalized) relative levels of Casq2, and/or Trdn, and/or Cav 1.2, and/or AnkB and/or RyR2, relative to non AAV-PKP2 treated, PKP2-cKO diseased controls.

### Example 2: In Vitro Testing of Adeno-Associated Virus Vectors

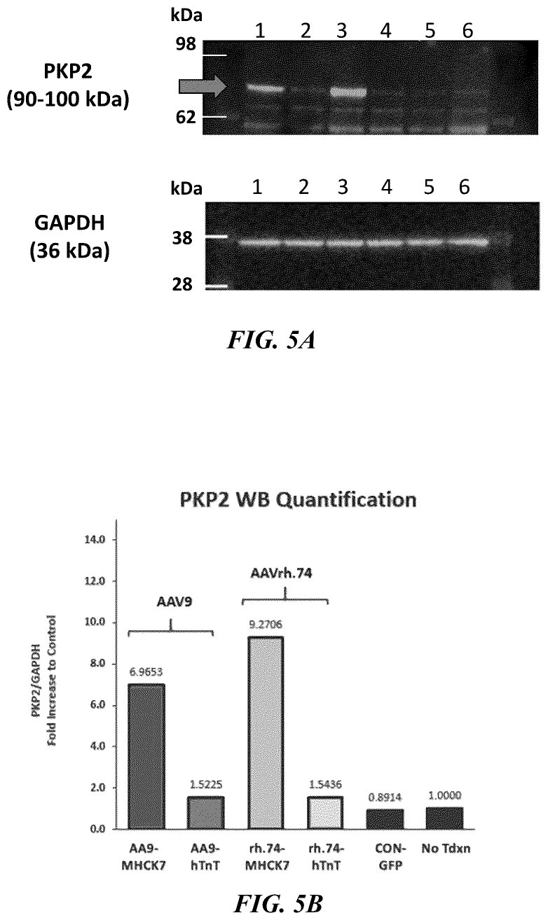

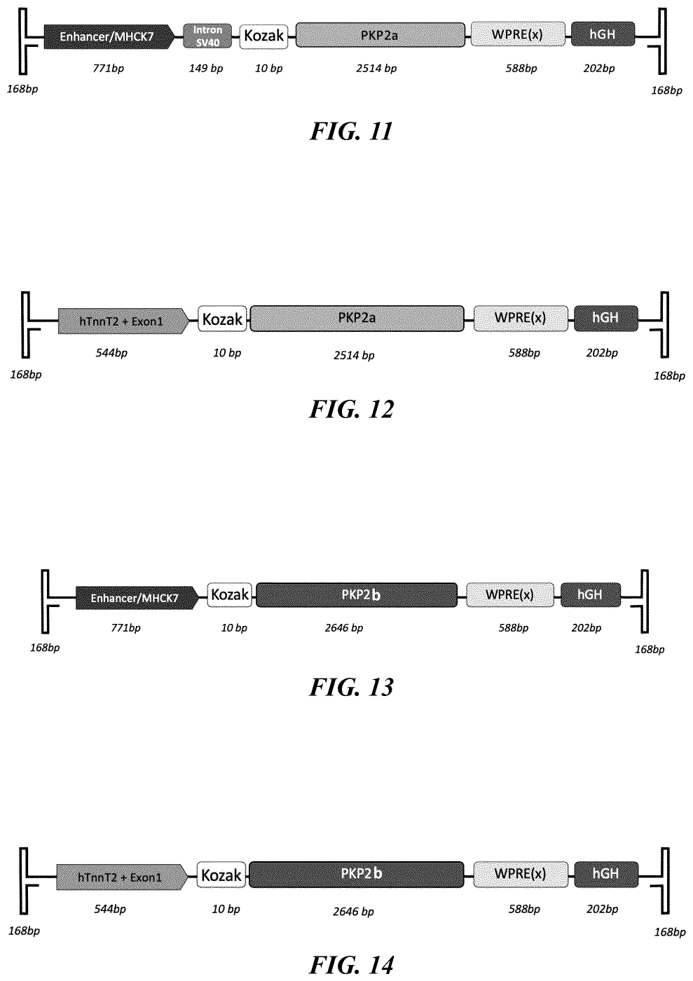

AAV vectors are described herein (see FIGS. 11-12) were prepared and used to transduce differentiated AC16 cells, a human cardiomyocyte cell line. Expression levels of PKP2 (PKP2a isoform) were assessed by Western Blot (FIGS. 5A-5B). Surprisingly, the MHCK7 promoter causes robust expression of PKP2 in cardiomyocytes, whereas the hTnnT2 promoter (“hTnT”) generates marginal PKP2 levels above background under the current testing conditions. The AAVrh.74 serotype induced higher expression of PKP2 than the AAV9 serotype vector.

Based on these results, we conclude that AAV9 vectors or AAVrh74 vectors can effectively be used to express PKP2 in cardiomyocytes, and that the MHCK7 promoter is superior to the hTnnT2 promoter when solely evaluating the relative levels of PKP2 expression.

### Example 3: In Vivo Efficacy of Adeno-Associated Virus Vectors

A “PKP2-cKO” mouse model of PKP2-deficiency, as described in Cerrone et al., Nat Comm., 2017 was obtained. This cardiomyocyte-specific, tamoxifen-activated PKP2 knockout murine line (aMHC-Cre-ER(T2)/Pkp2 fl/fl; referred to as “PKP2-cKO”) was utilized to control the onset of PKP2 loss of expression (see Cerrone et al., Nat Comm, 2017). The conditional loss of PKP2 expression in this mouse model is limited to adult myocytes and the temporal progression of the molecular, structural and functional events as a consequence of PKP2-cKO have been established (Cerrone et al., Nat Comm, 2017). PKP2 deficiency in adult ventricular myocytes is sufficient to cause an arrhythmogenic cardiomyopathy of RV predominance, which includes the ‘hallmark’ functional, molecular, and structural indices consistent with the disease phenotype of ARVC.

PKP2-Cko mice were injected with tamoxifen, causing myocyte-specific knockout of PKP2. Mice were injected with AAV vectors (as described below) at 3×10¹³ vg/kg by intravenous (tail vein) injection. Four weeks later, myocyte-specific knockout of PKP2 was induced by treatment of the mice with tamoxifen. The vector genomes used were:

5′ ITR; MHCK7 promoter (with its enhancer element); SV40 intron; Kozak sequence; PKPa transgene; WPRE(x); hGH polyadenylation sequence); 3′ ITR—shown in FIG. 11

5′ITR; hTnnT2 promoter (with exon 1); Kozak sequence; PKPa transgene; WPRE(x); hGH polyadenylation sequence); 3′ ITR—shown in FIG. 12.

Each vector genome was tested in a AAV9 serotype or AAVrh74 serotype vector.

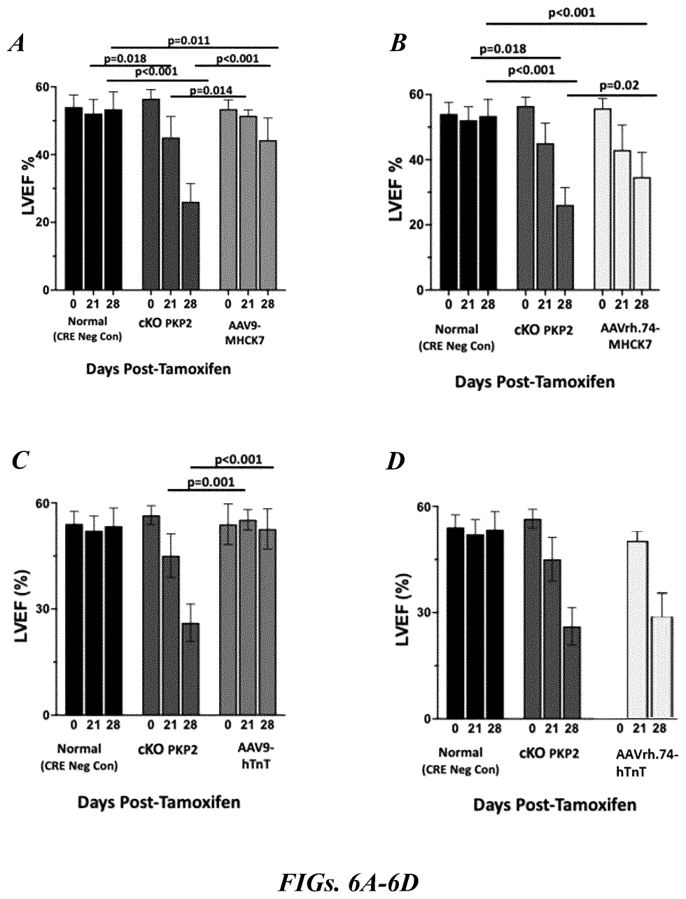

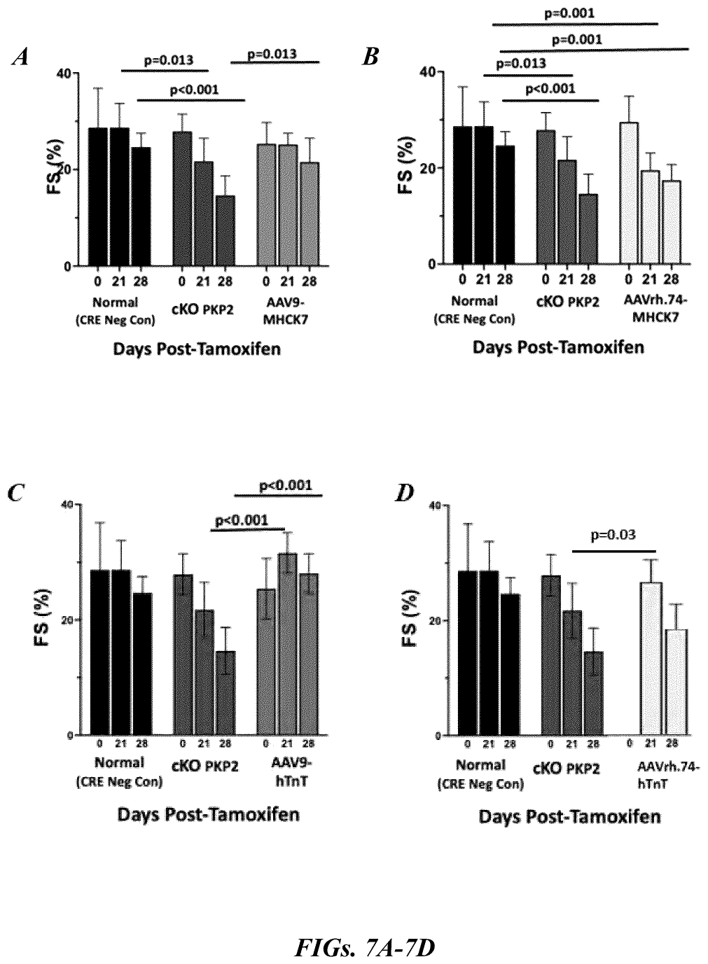

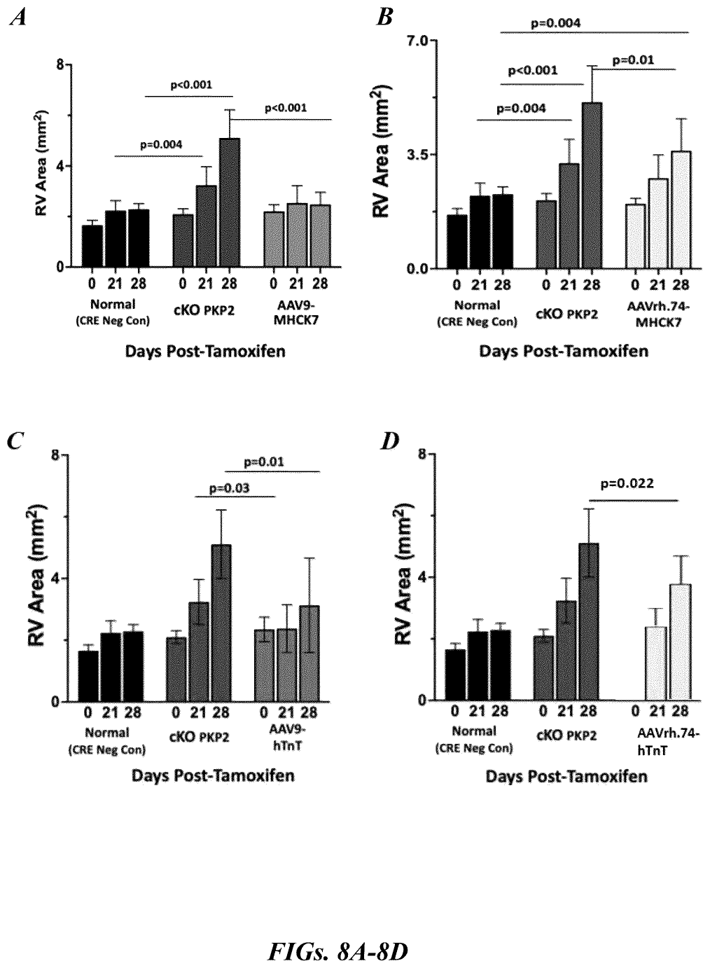

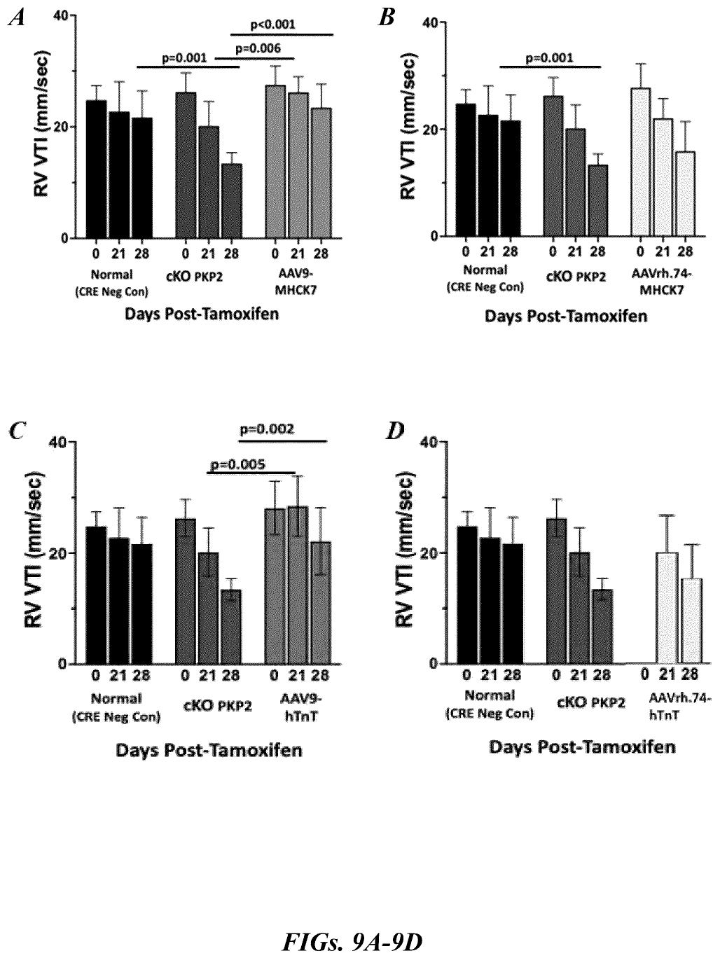

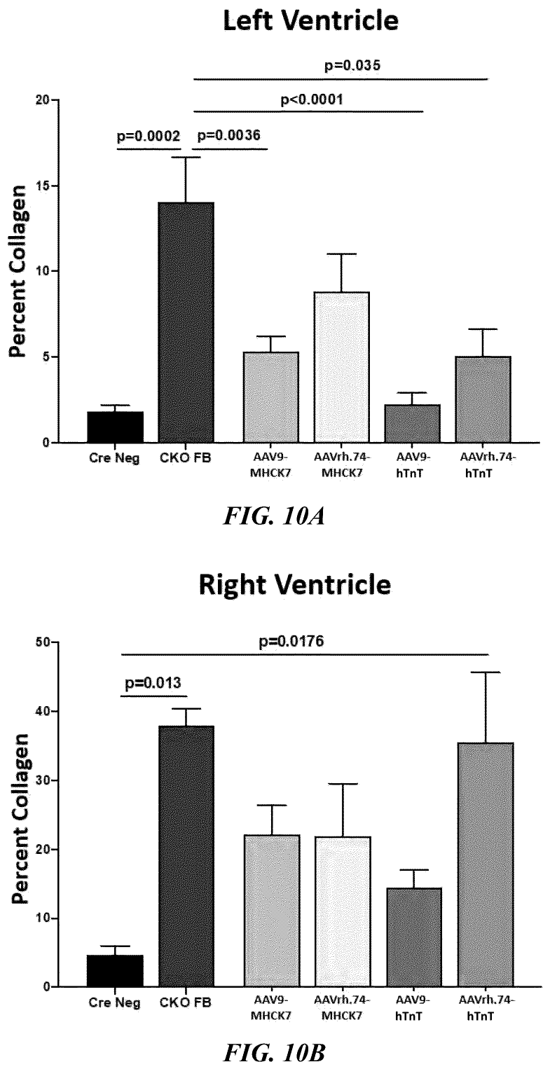

At 21 or 28 days after tamoxifen treatment, which is 25 or 32 weeks after AAV treatment, mice were evaluated for various physiology parameters, essentially as described in Cerrone et al., Nat Comm, 2017 or using standard methodologies known in the art. Efficacy in treating disease was assessed by left ventricle ejection fraction percentage (LVEF %) (FIGS. 6A-6D), left ventricle fractional shortening percentage (FS %) (FIGS. 7A-7D), right ventricle area in millimeters squared (RV Area (mm²)) (FIGS. 8A-8D), right ventricle velocity time integral in millimeters per second (RV VTI (mm/sec)) (FIGS. 9A-9D), and degree of fibrosis (FIGS. 10A-10B). These measures are appropriate functional and morphological indices to evaluate potential efficacy of AAV-mediated PKP2 overexpression in cardiomyocytes as they are among key parameters indicative of ARVC in human disease. Generally, a right ventricle normally has slightly greater amount of fibrosis (irrespective of disease); and this is further exacerbated with lack of PKP2 in the cKO model. Progressive deterioration of these parameters was observed within 21 days of tamoxifen injection, because tamoxifen injection causes myocyte-specific knockout of the PKP gene.

Evidence for mitigation of the disease phenotype was observed following both AAV9-and AAVrh.74-mediated PKP expression, to varying degrees. With the dose studied to date (3×10¹³ vg/kg) using a pre-treatment paradigm (AAV 4 weeks prior to tamoxifen-induced PKP cKO), AAV9 surprisingly produced the most robust effects on all parameters. Nevertheless, given the cardiotropism of AAVrh74 and given that biological effects were observed with AAVrh. 74-mediated overexpression of PKP2 in this model (e.g. LVEF %, FS %, and right ventricular area), optimization of the dose of AAVrh. 74 in combination with the appropriate promoter (i.e., either MHCK7 or hTnnT2) could enable robust therapeutic potential for this vector.

These results demonstrate both AAV9 and AAVrh.74 can be used to treat PKP2-related diseases, such as Arrhythmogenic right ventricular cardiomyopathy (ARVC) [also known as Arrhythmogenic Right Ventricular Dysplasia (ARVD) or Arrhythmogenic Cardiomyopathy (ACM)] for which the PKP-cKO mouse is considered an appropriate model. Additionally, vectors with either MHCK7 promoter or hTnnT2 promoter have been demonstrated to be effective in treating PKP2-related disease.

1-91. (canceled)

92. A method of treating arrhythmogenic cardiomyopathy (ACM) in a subject having a mutation in a PKP2 gene, comprising:
administering to the subject a recombinant adeno-associated virus (rAAV) vector comprising a vector genome,
wherein the vector genome comprises an expression cassette and flanking AAV inverted terminal repeats (ITRs), the expression cassette comprising a polynucleotide which comprises: a polynucleotide sequence encoding a Plakophilin-2 (PKP2); a promoter sequence, wherein the promoter sequence is operatively linked to the polynucleotide sequence encoding the PKP2, and wherein the promoter sequence shares at least 98% polynucleotide sequence identity with SEQ ID NO: 31; and a poly A sequence; and
wherein the rAAV vector is a recombinant AAV9 vector.

93. The method of claim 92, wherein the PKP2 is PKP2 isoform A, and comprises a polypeptide which shares at least 90% sequence identity with SEQ ID NO: 1.

94. The method of claim 92, wherein the recombinant AAV9 vector comprises a AAV9 capsid protein which shares at least 95% polypeptide sequence identity to SEQ ID NO: 77.

95. The method of claim 92, wherein the AAV9 capsid protein comprises the polypeptide sequence of SEQ ID NO: 77.

96. The method of claim 92, wherein the poly A sequence is a human growth hormone (hGH) poly A.

97. The method of claim 92, wherein the promoter sequence comprises a MHCK7 promoter.

98. The method of claim 92, wherein the promoter sequence comprises the polynucleotide sequence of SEQ ID NO: 31.

99. The method of claim 92, wherein the rAAV vector is administered by intravenous administration, intracoronary administration, intracardiac administration, or cardiac catheterization.

100. The method of claim 100, wherein the rAAV vector is administered by intravenous administration.

101. The method of claim 92, wherein the rAAV vector is administered at a dose of between about 1×10¹³ and 5×10¹⁴ vg/kg.

102. The method of claim 92, wherein the rAAV vector is administered at a dose of between about 5×10¹³ and 3×10¹⁴ vg/kg.

103. The method of claim 92, wherein the rAAV vector is administered at a dose of about 2×10¹⁴ vg/kg.

104. The method of claim 92, wherein the ACM is arrhythmogenic right ventricular cardiomyopathy (ARVC).
